SEQUENCE LISTING (249 sequences)

SEQ ID NO: 1 - DNA, 22 bp, Artificial sequence
  synthetic polynucleotide seqeuence
tccccagacgccgaggatggcc

SEQ ID NO: 2 - DNA, 22 bp, Artificial sequence
  synthetic polynucleotide seqeuence
tccccagaccccgaggatggcc

SEQ ID NO: 3 - DNA, 25 bp, Artificial sequence
  synthetic polynucleotide seqeuence
ccttggggattccccaactccgcag

SEQ ID NO: 4 - DNA, 24 bp, Artificial sequence
  synthetic polynucleotide seqeuence
cacatcagagccctgggcactgtc

SEQ ID NO: 5 - DNA, 27 bp, Artificial sequence
  synthetic polynucleotide seqeuence
ttatgcctacacgaacacagacacatg

SEQ ID NO: 6 - DNA, 22 bp, Artificial sequence
  synthetic polynucleotide seqeuence
ctcctcagacgccgagatgctg

SEQ ID NO: 7 - DNA, 22 bp, Artificial sequence
  synthetic polynucleotide seqeuence
ctcctcagacgccaagatgctg

SEQ ID NO: 8 - DNA, 22 bp, Artificial sequence
  synthetic polynucleotide seqeuence
ctcctcagacaccgagatgctg

SEQ ID NO: 9 - DNA, 22 bp, Artificial sequence
  synthetic polynucleotide seqeuence
ctcctcagacgccgagatgcgg

SEQ ID NO: 10 - DNA, 22 bp, Artificial sequence
  synthetic polynucleotide seqeuence
ctcctcagacgccaagatgcgg

SEQ ID NO: 11 - DNA, 22 bp, Artificial sequence
  synthetic polynucleotide seqeuence
ctcctcagacaccgagatgcgg

SEQ ID NO: 12 - DNA, 28 bp, Artificial sequence
  synthetic polynucleotide seqeuence
ccaacttgtgtcgggtccttcttccagg

SEQ ID NO: 13 - DNA, 28 bp, Artificial sequence
  synthetic polynucleotide seqeuence
ccaacctatgtcgggtccttcttccagg

SEQ ID NO: 14 - DNA, 24 bp, Artificial sequence
  synthetic polynucleotide seqeuence
cacatcagagccctgggcactgtc

SEQ ID NO: 15 - DNA, 26 bp, Artificial sequence
  synthetic polynucleotide seqeuence
catccctctttctacagcaaccccct

SEQ ID NO: 16 - DNA, 26 bp, Artificial sequence
  synthetic polynucleotide seqeuence
catccctctttcgacagcaaccccct

SEQ ID NO: 17 - DNA, 22 bp, Artificial sequence
  synthetic polynucleotide seqeuence
ctccccagacgccgagatgcgg

SEQ ID NO: 18 - DNA, 22 bp, Artificial sequence
  synthetic polynucleotide seqeuence
ctccccagaggccgagatgcgg

SEQ ID NO: 19 - DNA, 27 bp, Artificial sequence
  synthetic polynucleotide seqeuence
gagtccaaggggagaggtaagtttcct

SEQ ID NO: 20 - DNA, 27 bp, Artificial sequence
  synthetic polynucleotide seqeuence
gagtccaaggggagaggtaagtgtcct

SEQ ID NO: 21 - DNA, 24 bp, Artificial sequence
  synthetic polynucleotide seqeuence
ctcatcagagccctgggcactgtt

SEQ ID NO: 22 - DNA, 24 bp, Artificial sequence
  synthetic polynucleotide seqeuence
ctatccctcctcccacaccaaccg

SEQ ID NO: 23 - DNA, 31 bp, Artificial sequence
  synthetic polynucleotide seqeuence
gctcttaatacaaactcttcagctagtaact

SEQ ID NO: 24 - DNA, 31 bp, Artificial sequence; identical to SEQ ID NO: 23
  synthetic polynucleotide seqeuence

SEQ ID NO: 25 - DNA, 31 bp, Artificial sequence
  synthetic polynucleotide seqeuence
gctcttaatagaaactcttcaactagtaact

SEQ ID NO: 26 - DNA, 21 bp, Artificial sequence
  synthetic polynucleotide seqeuence
tcacaatggcccttggtgtct

SEQ ID NO: 27 - DNA, 21 bp, Artificial sequence
  synthetic polynucleotide seqeuence
tcacaatggcccctggtgtct

SEQ ID NO: 28 - DNA, 21 bp, Artificial sequence
  synthetic polynucleotide seqeuence
tcacaagggcccttggtgtct

SEQ ID NO: 29 - DNA, 32 bp, Artificial sequence
  synthetic polynucleotide seqeuence
ccatcaggtccgagctgtgttgactaccactt

SEQ ID NO: 30 - DNA, 32 bp, Artificial sequence
  synthetic polynucleotide seqeuence
ccatcaggtccgagctgtgttgactaccacta

SEQ ID NO: 31 - DNA, 32 bp, Artificial sequence
  synthetic polynucleotide seqeuence
ccatcaggtccaagctgtgttgactaccacta

SEQ ID NO: 32 - DNA, 32 bp, Artificial sequence
  synthetic polynucleotide seqeuence
ccatcaggtctgagctgtgttgactaccacta

SEQ ID NO: 33 - DNA, 32 bp, Artificial sequence
  synthetic polynucleotide seqeuence
ccatcaggtccgagctgtgttgactaccactg

SEQ ID NO: 34 - DNA, 25 bp, Artificial sequence
  synthetic polynucleotide seqeuence
cctagggcagagcagggggacaagc

SEQ ID NO: 35 - DNA, 25 bp, Artificial sequence
  synthetic polynucleotide seqeuence
cctagggcagagcagggagacaagc

SEQ ID NO: 36 - DNA, 25 bp, Artificial sequence
  synthetic polynucleotide seqeuence
cctagggcagagcagggggacaagc

SEQ ID NO: 37 - DNA, 23 bp, Artificial sequence
  synthetic polynucleotide seqeuence
agtcttgatcctcatagcagcaa

SEQ ID NO: 38 - DNA, 28 bp, Artificial sequence
  synthetic polynucleotide seqeuence
atgcagcggaccatgtgtcaacttatgc

SEQ ID NO: 39 - DNA, 29 bp, Artificial sequence
  synthetic polynucleotide seqeuence
acattcccacctttacagtatttcacagg

SEQ ID NO: 40 - DNA, 23 bp, Artificial sequence
  synthetic polynucleotide seqeuence
cgccccctccccgcagagaatta

SEQ ID NO: 41 - DNA, 28 bp, Artificial sequence
  synthetic polynucleotide seqeuence
acctttcttgctcctcctgtgcatgaag

SEQ ID NO: 42 - DNA, 23 bp, Artificial sequence
  synthetic polynucleotide seqeuence
ttcgtgtccccacagcacgtttc

SEQ ID NO: 43 - DNA, 23 bp, Artificial sequence
  synthetic polynucleotide seqeuence
ttcgtgtacccgcagcacgtttc

SEQ ID NO: 44 - DNA, 23 bp, Artificial sequence
  synthetic polynucleotide seqeuence
ttcgtgtccccacagcatgtttc

SEQ ID NO: 45 - DNA, 23 bp, Artificial sequence
  synthetic polynucleotide seqeuence
ttcttgtccccccagcacgtttc

SEQ ID NO: 46 - DNA, 23 bp, Artificial sequence
  synthetic polynucleotide seqeuence
tttgtgcccccacagcacgtttc

SEQ ID NO: 47 - DNA, 26 bp, Artificial sequence
  synthetic polynucleotide seqeuence
acctgttggctgaagtccagagtgtc

SEQ ID NO: 48 - DNA, 26 bp, Artificial sequence
  synthetic polynucleotide seqeuence
acctcttggctgaagtccagagtgtc

SEQ ID NO: 49 - DNA, 26 bp, Artificial sequence
  synthetic polynucleotide seqeuence
acctgttggctggagtccagagtgtc

SEQ ID NO: 50 - DNA, 26 bp, Artificial sequence
  synthetic polynucleotide seqeuence
acctgttggcggaagtccagagtgtc

SEQ ID NO: 51 - DNA, 26 bp, Artificial sequence
  synthetic polynucleotide seqeuence
acctgttgggtgaagtccagagtgcc

SEQ ID NO: 52 - DNA, 25 bp, Artificial sequence
  synthetic polynucleotide seqeuence
tgtgcgttggggacaaggcaattct

SEQ ID NO: 53 - DNA, 25 bp, Artificial sequence
  synthetic polynucleotide seqeuence
acacatcggaatcacctagggaact

SEQ ID NO: 54 - DNA, 25 bp, Artificial sequence
  synthetic polynucleotide seqeuence
gggtagaagatggtagatgacagct

SEQ ID NO: 55 - DNA, 25 bp, Artificial sequence
  synthetic polynucleotide seqeuence
gtggggaaaggaccccggtccctgc

SEQ ID NO: 56 - DNA, 25 bp, Artificial sequence
  synthetic polynucleotide seqeuence
acccttacactctctgccatgacca

SEQ ID NO: 57 - DNA, 25 bp, Artificial sequence
  synthetic polynucleotide seqeuence
aaacagggcccagccagggtccctc

SEQ ID NO: 58 - DNA, 25 bp, Artificial sequence
  synthetic polynucleotide seqeuence
gtgctgtgcaacagataatgactgc

SEQ ID NO: 59 - DNA, 25 bp, Artificial sequence
  synthetic polynucleotide seqeuence
aggaagtgaaaagtggtcaagctga

SEQ ID NO: 60 - DNA, 25 bp, Artificial sequence
  synthetic polynucleotide seqeuence
tgccaccgtcaccactatctacttg

SEQ ID NO: 61 - DNA, 25 bp, Artificial sequence
  synthetic polynucleotide seqeuence
taccatcaggaaggttcaaaccatg

SEQ ID NO: 62 - DNA, 25 bp, Artificial sequence
  synthetic polynucleotide seqeuence
ggtagaagatggtaggtgatggctg

SEQ ID NO: 63 - DNA, 25 bp, Artificial sequence
  synthetic polynucleotide seqeuence
gaaatggacacagttcctgatcctg

SEQ ID NO: 64 - DNA, 25 bp, Artificial sequence
  synthetic polynucleotide seqeuence
tctccctgaaaccgcttctaaatgc

SEQ ID NO: 65 - DNA, 25 bp, Artificial sequence
  synthetic polynucleotide seqeuence
gttgaggggaagccttctctgtcac

SEQ ID NO: 66 - DNA, 25 bp, Artificial sequence
  synthetic polynucleotide seqeuence
ctccacacccctctccagacactga

SEQ ID NO: 67 - DNA, 25 bp, Artificial sequence
  synthetic polynucleotide seqeuence
tttatgtggggaagggaagccttta

SEQ ID NO: 68 - DNA, 25 bp, Artificial sequence
  synthetic polynucleotide seqeuence
agtgaatggggaaggaatgagagac

SEQ ID NO: 69 - DNA, 25 bp, Artificial sequence
  synthetic polynucleotide seqeuence
ccttggggattccccaactccgcag

SEQ ID NO: 70 - DNA, 22 bp, Artificial sequence
  synthetic polynucleotide seqeuence
gaagagggatcaggacgaagtc

SEQ ID NO: 71 - DNA, 27 bp, Artificial sequence
  synthetic polynucleotide seqeuence
gagtccaaggggagaggtaagtttcct

SEQ ID NO: 72 - DNA, 27 bp, Artificial sequence
  synthetic polynucleotide seqeuence
gagtccaaggggagaggtaagtgtcct

SEQ ID NO: 73 - DNA, 28 bp, Artificial sequence
  synthetic polynucleotide seqeuence
atgcagcggaccatgtgtcaacttatgc

SEQ ID NO: 74 - DNA, 23 bp, Artificial sequence
  synthetic polynucleotide seqeuence
cgccccctccccgcagagaatta

SEQ ID NO: 75 - DNA, 19 bp, Artificial sequence
  synthetic polynucleotide seqeuence
ccccgtctccttccagggc

SEQ ID NO: 76 - DNA, 19 bp, Artificial sequence
  synthetic polynucleotide seqeuence
ccctgtctccttccagggc

SEQ ID NO: 77 - DNA, 28 bp, Artificial sequence
  synthetic polynucleotide seqeuence
tgccaggtacatcagatccatcaggtcc

SEQ ID NO: 78 - DNA, 28 bp, Artificial sequence
  synthetic polynucleotide seqeuence
tgccaggtacatcagatccatcaggtca

SEQ ID NO: 79 - DNA, 28 bp, Artificial sequence
  synthetic polynucleotide seqeuence
tgccaggtacatcagatccatcaggtct

SEQ ID NO: 80 - DNA, 28 bp, Artificial sequence
  synthetic polynucleotide seqeuence
tgccagctacatcagatccatcaggtcc

SEQ ID NO: 81 - DNA, 24 bp, Artificial sequence
  synthetic polynucleotide seqeuence
acctgaaagatcacggtgccttca

SEQ ID NO: 82 - DNA, 24 bp, Artificial sequence
  synthetic polynucleotide seqeuence
gcctgaaagatcccggtgccttca

SEQ ID NO: 83 - DNA, 24 bp, Artificial sequence
  synthetic polynucleotide seqeuence
acctgaaagatcatggtgccttca

SEQ ID NO: 84 - DNA, 24 bp, Artificial sequence
  synthetic polynucleotide seqeuence
gaggtctccagaacaggctggagg

SEQ ID NO: 85 - DNA, 24 bp, Artificial sequence
  synthetic polynucleotide seqeuence
gaggtctccagaaccggctggagg

SEQ ID NO: 86 - DNA, 24 bp, Artificial sequence
  synthetic polynucleotide seqeuence
gagatctccagaacaggctggagg

SEQ ID NO: 87 - DNA, 24 bp, Artificial sequence
  synthetic polynucleotide seqeuence
gagttctccagaacaggctggagg

SEQ ID NO: 88 - DNA, 28 bp, Artificial sequence
  synthetic polynucleotide seqeuence
tgccaggtacatcagatccatcaggtcc

SEQ ID NO: 89 - DNA, 28 bp, Artificial sequence
  synthetic polynucleotide seqeuence
tgccaggtacatcagatccatcaggtca

SEQ ID NO: 90 - DNA, 28 bp, Artificial sequence
  synthetic polynucleotide seqeuence
tgccaggtacatcagatccatcaggtct

SEQ ID NO: 91 - DNA, 28 bp, Artificial sequence
  synthetic polynucleotide seqeuence
tgccagctacatcagatccatcaggtcc

SEQ ID NO: 92 - DNA, 27 bp, Artificial sequence
  synthetic polynucleotide seqeuence
ttatgcctacacgaacacagacacatg

SEQ ID NO: 93 - DNA, 26 bp, Artificial sequence
  synthetic polynucleotide seqeuence
catccctctttctacagcaaccccct

SEQ ID NO: 94 - DNA, 26 bp, Artificial sequence
  synthetic polynucleotide seqeuence
catccctctttcgacagcaaccccct

SEQ ID NO: 95 - DNA, 24 bp, Artificial sequence
  synthetic polynucleotide seqeuence
ctatccctcctcccacaccaaccg

SEQ ID NO: 96 - DNA, 29 bp, Artificial sequence
  synthetic polynucleotide seqeuence
acattcccacctttacagtatttcacagg

SEQ ID NO: 97 - DNA, 28 bp, Artificial sequence
  synthetic polynucleotide seqeuence
acctttcttgctcctcctgtgcatgaag

SEQ ID NO: 98 - DNA, 19 bp, Artificial sequence
  synthetic polynucleotide seqeuence
gcgatgcacctgcaacagg

SEQ ID NO: 99 - DNA, 24 bp, Artificial sequence
  synthetic polynucleotide seqeuence
agtcttgatcctcatagcagcaaa

SEQ ID NO: 100 - DNA, 23 bp, Artificial sequence
  synthetic polynucleotide seqeuence
gaaacgtgctgtggggacacgaa

SEQ ID NO: 101 - DNA, 23 bp, Artificial sequence
  synthetic polynucleotide seqeuence
gaaacgtgctgcgggtacacgaa

SEQ ID NO: 102 - DNA, 23 bp, Artificial sequence
  synthetic polynucleotide seqeuence
gaaacatgctgtggggacacgaa

SEQ ID NO: 103 - DNA, 23 bp, Artificial sequence
  synthetic polynucleotide seqeuence
gaaacgtgctggggggacaagaa

SEQ ID NO: 104 - DNA, 23 bp, Artificial sequence
  synthetic polynucleotide seqeuence
gaaacgtgctgtgggggcacaaa

SEQ ID NO: 105 - DNA, 27 bp, Artificial sequence
  synthetic polynucleotide seqeuence
gtcatctgcatttcagctcaggaatcc

SEQ ID NO: 106 - DNA, 27 bp, Artificial sequence
  synthetic polynucleotide seqeuence
gtcatctgcacttcagctcaagagtcc

SEQ ID NO: 107 - DNA, 27 bp, Artificial sequence
  synthetic polynucleotide seqeuence
gtcatctgcacttcagctcaggaatcc

SEQ ID NO: 108 - DNA, 27 bp, Artificial sequence
  synthetic polynucleotide seqeuence
gtcatcttcacttcagctcaggaatcc

SEQ ID NO: 109 - DNA, 28 bp, Artificial sequence
  synthetic polynucleotide seqeuence
agtcttgatcctcatagcagcaaatagg

SEQ ID NO: 110 - DNA, 28 bp, Artificial sequence
  synthetic polynucleotide seqeuence
agtcttgatcctcatagcagcaaatata

SEQ ID NO: 111 - DNA, 25 bp, Artificial sequence
  synthetic polynucleotide seqeuence
ccttggggattccccaactccgcag

SEQ ID NO: 112 - DNA, 28 bp, Artificial sequence
  synthetic polynucleotide seqeuence
ccaacttgtgtcgggtccttcttccagg

SEQ ID NO: 113 - DNA, 28 bp, Artificial sequence
  synthetic polynucleotide seqeuence
ccaacctatgtcgggtccttcttccagg

SEQ ID NO: 114 - DNA, 28 bp, Artificial sequence
  synthetic polynucleotide seqeuence
ccaacttgtgtcgggtccttcttccagg

SEQ ID NO: 115 - DNA, 28 bp, Artificial sequence
  synthetic polynucleotide seqeuence
ccaacctatgtcgggtccttcttccagg

SEQ ID NO: 116 - DNA, 22 bp, Artificial sequence
  synthetic polynucleotide seqeuence
gaagagggatcaggacgaagtc

SEQ ID NO: 117 - DNA, 27 bp, Artificial sequence
  synthetic polynucleotide seqeuence
gagtccaaggggagaggtaagtttcct

SEQ ID NO: 118 - DNA, 27 bp, Artificial sequence
  synthetic polynucleotide seqeuence
gagtccaaggggagaggtaagtgtcct

SEQ ID NO: 119 - DNA, 28 bp, Artificial sequence
  synthetic polynucleotide seqeuence
atgcagcggaccatgtgtcaacttatgc

SEQ ID NO: 120 - DNA, 25 bp, Artificial sequence
  synthetic polynucleotide seqeuence
cactgttcctgtgctcacagtcatc

SEQ ID NO: 121 - DNA, 23 bp, Artificial sequence
  synthetic polynucleotide seqeuence
cgccccctccccgcagagaatta

SEQ ID NO: 122 - DNA, 19 bp, Artificial sequence
  synthetic polynucleotide seqeuence
ccccgtctccttccagggc

SEQ ID NO: 123 - DNA, 19 bp, Artificial sequence
  synthetic polynucleotide seqeuence
ccctgtctccttccagggc

SEQ ID NO: 124 - DNA, 22 bp, Artificial sequence
  synthetic polynucleotide seqeuence
ttgccccgtctccttccagggc

SEQ ID NO: 125 - DNA, 22 bp, Artificial sequence
  synthetic polynucleotide seqeuence
ttgccctgtctccttccagggc

SEQ ID NO: 126 - DNA, 28 bp, Artificial sequence
  synthetic polynucleotide seqeuence
tgccaggtacatcagatccatcaggtcc

SEQ ID NO: 127 - DNA, 28 bp, Artificial sequence
  synthetic polynucleotide seqeuence
tgccaggtacatcagatccatcaggtca

SEQ ID NO: 128 - DNA, 28 bp, Artificial sequence
  synthetic polynucleotide seqeuence
tgccaggtacatcagatccatcaggtct

SEQ ID NO: 129 - DNA, 28 bp, Artificial sequence
  synthetic polynucleotide seqeuence
tgccagctacatcagatccatcaggtcc

SEQ ID NO: 130 - DNA, 25 bp, Artificial sequence
  synthetic polynucleotide seqeuence
caggtacatcagatccatcaggtcc

SEQ ID NO: 131 - DNA, 25 bp, Artificial sequence
  synthetic polynucleotide seqeuence
caggtacatcagatccatcaggtca

SEQ ID NO: 132 - DNA, 25 bp, Artificial sequence
  synthetic polynucleotide seqeuence
caggtacatcagatccatcaggtct

SEQ ID NO: 133 - DNA, 25 bp, Artificial sequence
  synthetic polynucleotide seqeuence
cagctacatcagatccatcaggtcc

SEQ ID NO: 134 - DNA, 25 bp, Artificial sequence
  synthetic polynucleotide seqeuence
tacatcagatccatcaggtccgagc

SEQ ID NO: 135 - DNA, 25 bp, Artificial sequence
  synthetic polynucleotide seqeuence
tacatcagatccatcaggtccaagc

SEQ ID NO: 136 - DNA, 25 bp, Artificial sequence
  synthetic polynucleotide seqeuence
tacatcagatccatcaggtctgagc

SEQ ID NO: 137 - DNA, 25 bp, Artificial sequence
  synthetic polynucleotide seqeuence
gtccgagctgtgttgactaccactt

SEQ ID NO: 138 - DNA, 25 bp, Artificial sequence
  synthetic polynucleotide seqeuence
gtccgagctgtgttgactaccacta

SEQ ID NO: 139 - DNA, 25 bp, Artificial sequence
  synthetic polynucleotide seqeuence
gtccaagctgtgttgactaccacta

SEQ ID NO: 140 - DNA, 25 bp, Artificial sequence
  synthetic polynucleotide seqeuence
gtctgagctgtgttgactaccacta

SEQ ID NO: 141 - DNA, 25 bp, Artificial sequence
  synthetic polynucleotide seqeuence
gtccgagctgtgttgactaccactg

SEQ ID NO: 142 - DNA, 25 bp, Artificial sequence
  synthetic polynucleotide seqeuence
caggtacatcagatccatcaggtcc

SEQ ID NO: 143 - DNA, 25 bp, Artificial sequence
  synthetic polynucleotide seqeuence
caggtacatcagatccatcaggtca

SEQ ID NO: 144 - DNA, 25 bp, Artificial sequence
  synthetic polynucleotide seqeuence
caggtacatcagatccatcaggtct

SEQ ID NO: 145 - DNA, 25 bp, Artificial sequence
  synthetic polynucleotide seqeuence
cagctacatcagatccatcaggtcc

SEQ ID NO: 146 - DNA, 24 bp, Artificial sequence
  synthetic polynucleotide seqeuence
gcctgaaagatcccggtgccttca

SEQ ID NO: 147 - DNA, 24 bp, Artificial sequence
  synthetic polynucleotide seqeuence
acctgaaagatcatggtgccttca

SEQ ID NO: 148 - DNA, 24 bp, Artificial sequence
  synthetic polynucleotide seqeuence
acctgaaagatcacggtgccttca

SEQ ID NO: 149 - DNA, 24 bp, Artificial sequence
  synthetic polynucleotide seqeuence
gcctgaaagatcccggtgccttca

SEQ ID NO: 150 - DNA, 24 bp, Artificial sequence
  synthetic polynucleotide seqeuence
acctgaaagatcatggtgccttca

SEQ ID NO: 151 - DNA, 24 bp, Artificial sequence
  synthetic polynucleotide seqeuence
gaggtctccagaacaggctggagg

SEQ ID NO: 152 - DNA, 24 bp, Artificial sequence
  synthetic polynucleotide seqeuence
gaggtctccagaaccggctggagg

SEQ ID NO: 153 - DNA, 24 bp, Artificial sequence
  synthetic polynucleotide seqeuence
gagatctccagaacaggctggagg

SEQ ID NO: 154 - DNA, 24 bp, Artificial sequence
  synthetic polynucleotide seqeuence
gagttctccagaacaggctggagg

SEQ ID NO: 155 - DNA, 24 bp, Artificial sequence
  synthetic polynucleotide seqeuence
acctgaaagatcacggtgccttca

SEQ ID NO: 156 - DNA, 24 bp, Artificial sequence
  synthetic polynucleotide seqeuence
gcctgaaagatcccggtgccttca

SEQ ID NO: 157 - DNA, 24 bp, Artificial sequence
  synthetic polynucleotide seqeuence
acctgaaagatcatggtgccttca

SEQ ID NO: 158 - DNA, 27 bp, Artificial sequence
  synthetic polynucleotide seqeuence
ttatgcctacacgaacacagacacatg

SEQ ID NO: 159 - DNA, 26 bp, Artificial sequence
  synthetic polynucleotide seqeuence
catccctctttctacagcaaccccct

SEQ ID NO: 160 - DNA, 26 bp, Artificial sequence
  synthetic polynucleotide seqeuence
catccctctttcgacagcaaccccct

SEQ ID NO: 161 - DNA, 28 bp, Artificial sequence
  synthetic polynucleotide seqeuence
cccatccctctttctacagcaaccccct

SEQ ID NO: 162 - DNA, 26 bp, Artificial sequence
  synthetic polynucleotide seqeuence
catccctctttctacagcaaccccct

SEQ ID NO: 163 - DNA, 26 bp, Artificial sequence
  synthetic polynucleotide seqeuence
catccctctttcgacagcaaccccct

SEQ ID NO: 164 - DNA, 26 bp, Artificial sequence
  synthetic polynucleotide seqeuence
catccctctttctacagcaaccccct

SEQ ID NO: 165 - DNA, 26 bp, Artificial sequence
  synthetic polynucleotide seqeuence
catccctctttcgacagcaaccccct

SEQ ID NO: 166 - DNA, 24 bp, Artificial sequence
  synthetic polynucleotide seqeuence
ctatccctcctcccacaccaaccg

SEQ ID NO: 167 - DNA, 29 bp, Artificial sequence
  synthetic polynucleotide seqeuence
acattcccacctttacagtatttcacagg

SEQ ID NO: 168 - DNA, 29 bp, Artificial sequence
  synthetic polynucleotide seqeuence
acattcccacctttacagtatttcacagg

SEQ ID NO: 169 - DNA, 28 bp, Artificial sequence
  synthetic polynucleotide seqeuence
acctttcttgctcctcctgtgcatgaag

SEQ ID NO: 170 - DNA, 19 bp, Artificial sequence
  synthetic polynucleotide seqeuence
gcgatgcacctgcaacagg

SEQ ID NO: 171 - DNA, 23 bp, Artificial sequence
  synthetic polynucleotide seqeuence
gatggcgatgcacctgcaacagg

SEQ ID NO: 172 - DNA, 24 bp, Artificial sequence
  synthetic polynucleotide seqeuence
agtcttgatcctcatagcagcaaa

SEQ ID NO: 173 - DNA, 24 bp, Artificial sequence
  synthetic polynucleotide seqeuence
agtcttgatcctcatagcagcaaa

SEQ ID NO: 174 - DNA, 24 bp, Artificial sequence
  synthetic polynucleotide seqeuence
agtcttgatcctcatagcagcaaa

SEQ ID NO: 175 - DNA, 24 bp, Artificial sequence
  synthetic polynucleotide seqeuence
agtcttgatcctcatagcagcaaa

SEQ ID NO: 176 - DNA, 28 bp, Artificial sequence
  synthetic polynucleotide seqeuence
agtcttgatcctcatagcagcaaatagg

SEQ ID NO: 177 - DNA, 28 bp, Artificial sequence
  synthetic polynucleotide seqeuence
agtcttgatcctcatagcagcaaatata

SEQ ID NO: 178 - DNA, 26 bp, Artificial sequence
  synthetic polynucleotide seqeuence
tcttgatcctcatagcagcaaatagg

SEQ ID NO: 179 - DNA, 26 bp, Artificial sequence
  synthetic polynucleotide seqeuence
tcttgatcctcatagcagcaaatata

SEQ ID NO: 180 - DNA, 25 bp, Artificial sequence
  synthetic polynucleotide seqeuence
cctctaaagaccctgaggacatgtg

SEQ ID NO: 181 - DNA, 25 bp, Artificial sequence
  synthetic polynucleotide seqeuence
cctctaaagaccctgagtacatgtg

SEQ ID NO: 182 - DNA, 24 bp, Artificial sequence
  synthetic polynucleotide seqeuence
cctccagcctgttctggagacctc

SEQ ID NO: 183 - DNA, 24 bp, Artificial sequence
  synthetic polynucleotide seqeuence
cctccagccggttctggagacctc

SEQ ID NO: 184 - DNA, 24 bp, Artificial sequence
  synthetic polynucleotide seqeuence
cctccagcctgttctggagatctc

SEQ ID NO: 185 - DNA, 24 bp, Artificial sequence
  synthetic polynucleotide seqeuence
cctccagcctgttctggagaactc

SEQ ID NO: 186 - DNA, 27 bp, Artificial sequence
  synthetic polynucleotide seqeuence
gtcatctgcatttcagctcaggaatcc

SEQ ID NO: 187 - DNA, 27 bp, Artificial sequence
  synthetic polynucleotide seqeuence
gtcatctgcacttcagctcaagagtcc

SEQ ID NO: 188 - DNA, 27 bp, Artificial sequence
  synthetic polynucleotide seqeuence
gtcatctgcacttcagctcaggaatcc

SEQ ID NO: 189 - DNA, 27 bp, Artificial sequence
  synthetic polynucleotide seqeuence
gtcatcttcacttcagctcaggaatcc

SEQ ID NO: 190 - DNA, 23 bp, Artificial sequence
  synthetic polynucleotide seqeuence
gaaacgtgctgtggggacacgaa

SEQ ID NO: 191 - DNA, 23 bp, Artificial sequence
  synthetic polynucleotide seqeuence
gaaacgtgctgcgggtacacgaa

SEQ ID NO: 192 - DNA, 23 bp, Artificial sequence
  synthetic polynucleotide seqeuence
gaaacatgctgtggggacacgaa

SEQ ID NO: 193 - DNA, 23 bp, Artificial sequence
  synthetic polynucleotide seqeuence
gaaacgtgctggggggacaagaa

SEQ ID NO: 194 - DNA, 23 bp, Artificial sequence
  synthetic polynucleotide seqeuence
gaaacgtgctgtgggggcacaaa

SEQ ID NO: 195 - DNA, 62 bp, Artificial sequence
  synthetic polynucleotide seqeuence
ttgtcttgtggacaacatctttcctcctgtggtcaacatcacatggctgagcaatgggcagt

SEQ ID NO: 196 - DNA, 62 bp, Artificial sequence
  synthetic polynucleotide seqeuence
ctgtcttgtggacaacatctttcctcctgtggtcaacatcacatggctgagcaatgggcagt

SEQ ID NO: 197 - DNA, 62 bp, Artificial sequence
  synthetic polynucleotide seqeuence
ctgtcttgtggacaacatctttcctcctgtggtcaacatcacatggctgagcaatgggcacg

SEQ ID NO: 198 - DNA, 103 bp, Artificial sequence
  synthetic polynucleotide seqeuence
ccggcccggccgcggggagccccgcttcatcgcagtgggctacgtggacgacacgcagttcgtgcggttcgacagcgacgccgcgagccagaggatggagccg

SEQ ID NO: 199 - DNA, 108 bp, Artificial sequence
  synthetic polynucleotide seqeuence
ccggcccggccgcggggagccccgcttcatcgcagtgggctacgtggactggacgacacgcagttcgtgcggttcgacagcgacgccgcgagccagaggatggagccg

SEQ ID NO: 200 - DNA, 102 bp, Artificial sequence
  synthetic polynucleotide seqeuence
aggagccgcgggcgccatggatagagcaggaggggccggagtattgggaccgggagacacagatctccaagaccaacacacagacttaccgagagagcctgc

SEQ ID NO: 201 - DNA, 110 bp, Artificial sequence
  synthetic polynucleotide seqeuence
aggagccgcgggcgccatggatagagcaggaggggccggagtattgggaccgggagacacagatctccaagaccaacacacagacttaccgagttaccgagagagcctgc

SEQ ID NO: 202 - DNA, 108 bp, Artificial sequence
  synthetic polynucleotide seqeuence
gtgcgtggagtggctccgcagacacctggagaacgggaaggagacgctgcagcgcgcgggtaccaggggcagtggggagccttccccatctcctataggtcgccgggg

SEQ ID NO: 203 - DNA, 107 bp, Artificial sequence
  synthetic polynucleotide seqeuence
gtgcgtggagtggctccgcagacacctggagaacgggaaggagacgctgcagcgcgcgggtaccggggcagtggggagccttccccatctcctataggtcgccgggg

SEQ ID NO: 204 - DNA, 174 bp, Artificial sequence
  synthetic polynucleotide seqeuence
ggacctcctggagcagaggcgggccgcggtggacacctactgcagacacaactacggggttggtgagagcttcacagtgcagcggcgaggtgagcgcggcgcggggcggggcctgagtccctgtaagcggagaatctgagtgtgtgtgtgtgtgtgtgtgtgtgtgtgtgtaag

SEQ ID NO: 205 - DNA, 179 bp, Artificial sequence
  synthetic polynucleotide seqeuence
ggacttcctggaagacaggcgggccgcggtggacacctactgcagacacaactacggggttggtgagagcttcacagtgcagcggcgaggtgagcgcggcgcggggcggggcctgagtccctgtgagctgggaatctgagtgtgtgtgtgtgtgtgtgtgtgtgtgtgtgtgtgtgtgt

SEQ ID NO: 206 - DNA, 179 bp, Artificial sequence
  synthetic polynucleotide seqeuence
ggacttcctggaagacaggcgggccgcggtggacacctactgcagacacaactacggggttggtgagagcttcacggtgcagcggcgaggtgagcgcggcgcggggcggggcctgagtccctgtgagctgggaatctgagtgtgtgtgtgtgtgtgtgtgtgtgtgtgtgtgtgtgtgt

SEQ ID NO: 207 - DNA, 180 bp, Artificial sequence
  synthetic polynucleotide seqeuence
cgacagccgacgccgcgagtccgaggatggcgccccgggcgccatggatagagcaggaggggccggagtattgggaccgcaacacacagatctgcaagaccaacacacagacttaccgagagagcctgcggaacctgcgcggctactacaaccagagcgaggccgggtctcacatcatcc

SEQ ID NO: 208 - DNA, 180 bp, Artificial sequence
  synthetic polynucleotide seqeuence
cgacagccgacgccgcgagtccgaggatggcgccccgggcgccatggatagagcaggaggggccggagtattgggaccgcgagacacagatctccaagaccaacacacagacttaccgagagagcctgcggaacctgcgcggctactacaaccagagcgaggccgggtctcacaccctcc

SEQ ID NO: 209 - DNA, 448 bp, Artificial sequence
  synthetic polynucleotide seqeuence
atgcgggtcacggcgccccgaaccgtcctcctgctgctctcgggagccctggccctgaccgagacctgggccggctcccactccatgaggtatttctacaccgccatgtcccggcccggccgcggggagccccgcttcatcgcagtgggctacgtggacgacacccagttcgtgaggttcgacagcgacgccgcgagtccgaggatggcgccccgggcgccatggatagagcaggaggggccggagtattgggaccgggagacacagatctccaagaccaacacacagacttaccgagagagcctgcggaacctgcgcggctactacaaccagagcgaggccgggtctcacaccctccagaggatgtacggctgcgacgtggggccggacgggcgcctcctccgcgggcatgaccagtccgcctacgacggcaaggattacatcgccc

SEQ ID NO: 210 - DNA, 448 bp, Artificial sequence
  synthetic polynucleotide seqeuence
atgcgggtcatggcgccccgaaccgtcctcctgctgctctcgggagccctggccctgaccgagacctgggccggctcccactccatgaggtatttctacaccgccatgtcccggcccggccgcggggagccccgcttcatcgcagtgggctacgtggacgacacccagttcgtgaggttcgacagcgacgccgcgagtccgaggatggcgccccgggcgccatggatagagcaggaggggccggagtattgggaccgggagacacagatctccaagaccaacacacagacttaccgagagagcctgcggaacctgcgcggctactacaaccagagcgaggccgggtctcacaccctccagaggatgtaaggctgcgacgtggggccggacgggcgcctcctccgcgggcatgaccagtccgcctacgacggcaaggattacatcgccc

SEQ ID NO: 211 - DNA, 29 bp, Artificial sequence
  synthetic polynucleotide seqeuence
ttgtacagagacagcggggcgacgggaat

SEQ ID NO: 212 - DNA, 29 bp, Artificial sequence
  synthetic polynucleotide seqeuence
cgaacgtagaggcaacatagctgacaccg

SEQ ID NO: 213 - protein, 29 aa, Artificial sequence
  synthetic polynucleotide seqeuence
YKRWMSWRRRRSMRMSRKRSSWSRSRMMK

SEQ ID NO: 214 - DNA, 29 bp, Artificial sequence
  synthetic polynucleotide seqeuence
ttgtacagagacagcgggagctgacaccg

SEQ ID NO: 215 - DNA, 29 bp, Artificial sequence
  synthetic polynucleotide seqeuence
cgaacgtagaggcaacatgcgacgggaat

SEQ ID NO: 216 - DNA, 29 bp, Artificial sequence
  synthetic polynucleotide seqeuence
acccccccaagacacatatgacccaccac

SEQ ID NO: 217 - DNA, 21 bp, Artificial sequence
  synthetic polynucleotide seqeuence
aaaacgcatatgactcaccac

SEQ ID NO: 218 - DNA, 21 bp, Artificial sequence
  synthetic polynucleotide seqeuence
caagacacatatgacccacca

SEQ ID NO: 219 - protein, 12 aa, Artificial sequence
  synthetic polynucleotide seqeuence
MAARMSMMWWWK

SEQ ID NO: 220 - DNA, 21 bp, Artificial sequence
  synthetic polynucleotide seqeuence
aagacacatatgacccaccac

SEQ ID NO: 221 - DNA, 21 bp, Artificial sequence
  synthetic polynucleotide seqeuence
caaaacgcatatgactcacca

SEQ ID NO: 222 - protein, 12 aa, Artificial sequence
  synthetic polynucleotide seqeuence
MARAMMSMWWWK

SEQ ID NO: 223 - DNA, 58 bp, Artificial sequence
  synthetic polynucleotide seqeuence
caaaatgcctgaatgttctgactcttcctgacagacccccccaagacgcatatgactc

SEQ ID NO: 224 - DNA, 59 bp, Artificial sequence
  synthetic polynucleotide seqeuence
caaaatgcctgaatgttctgactcttcctgacagaccccccccaagacgcatatgactc

SEQ ID NO: 225 - DNA, 52 bp, Artificial sequence
  synthetic polynucleotide seqeuence
gacgggctccgcagatacctggagaacgggaaggagacgctgcagcgcacgg

SEQ ID NO: 226 - DNA, 768 bp, Artificial sequence
  synthetic polynucleotide seqeuence
atgatcctaaacaaagctctgctgctgggggccctcgctctgaccaccgtgatgagcccctgtggaggtgaagacattgtggctgaccacgttgcctcttgtggtgtaaacttgtaccagttttacggtccctctggccagtacacccatgaatttgatggagatgaggagttctacgtggacctggagaggaaggagactgcctggcggtggcctgagttcagcaaatttggaggttttgacccgcagggtgcactgagaaacatggctgtggcaaaacacaacttgaacatcatgattaaacgctacaactctaccgctgctaccaatgaggttcctgaggtcacagtgttttccaagtctcccgtgacactgggtcagcccaacaccctcatttgtcttgtggacaacatctttcctcctgtggtcaacatcacatggctgagcaatgggcagtcagtcacagaaggtgtttctgagaccagcttcctctccaagagtgatcattccttcttcaagatcagttacctcaccttcctcccttctgctgatgagatttatgactgcaaggtggagcactggggcctggaccagcctcttctgaaacactgggagcctgagattccagcccctatgtcagagctcacagagactgtggtctgcgccctggggttgtctgtgggcctcgtgggcattgtggtgggcactgtcttcatcatccaaggcctgcgttcagttggtgcttccagacaccaagggccattgtga

SEQ ID NO: 227 - DNA, 801 bp, Artificial sequence
  synthetic polynucleotide seqeuence
atggtgtgtctgaagctccctggaggctcctgcatgacagcgctgacagtgacactgatggtgctgagctccccactggctttggctggggacacccgaccacgtttcttgtggcagcttaagtttgaatgtcatttcttcaatgggacggagcgggtgcggttgctggaaagatgcatctataaccaagaggagtccgtgcgcttcgacagcgacgtgggggagtaccgggcggtgacggagctggggcggcctgatgccgagtactggaacagccagaaggacctcctggagcagaggcgggccgcggtggacacctactgcagacacaactacggggttggtgagagcttcacagtgcagcggcgagttgagcctaaggtgactgtgtatccttcaaagacccagcccctgcagcaccacaacctcctggtctgctctgtgagtggtttctatccaggcagcattgaagtcaggtggttccggaacggccaggaagagaaggctggggtggtgtccacaggcctgatccagaatggagattggaccttccagaccctggtgatgctggaaacagttcctcggagtggagaggtttacacctgccaagtggagcacccaagtgtgacgagccctctcacagtggaatggagagcacggtctgaatctgcacagagcaagatgctgagtggagtcgggggcttcgtgctgggcctgctcttccttggggccgggctgttcatctacttcaggaatcagaaaggacactctggacttcagccaacaggattcctgagctga

SEQ ID NO: 228 - DNA, 31 bp, Artificial sequence
  synthetic polynucleotide seqeuence
tggaccgccgcggacaccgcggctcagatca

SEQ ID NO: 229 - DNA, 31 bp, Artificial sequence
  synthetic polynucleotide seqeuence
tggaccgccgcggacacggcggctcagatca

SEQ ID NO: 230 - DNA, 50 bp, Artificial sequence
  synthetic polynucleotide seqeuence
agaagagaaattacacatactgtgaaactcatagtgccggcaagtactta

SEQ ID NO: 231 - DNA, 50 bp, Artificial sequence
  synthetic polynucleotide seqeuence
agaagagaaattatacatactgtgaaactcatagtgccagcaagtactta

SEQ ID NO: 232 - DNA, 62 bp, Artificial sequence; identical to SEQ ID NO: 195
  synthetic polynucleotide seqeuence

SEQ ID NO: 233 - DNA, 62 bp, Artificial sequence; identical to SEQ ID NO: 196
  synthetic polynucleotide seqeuence

SEQ ID NO: 234 - DNA, 62 bp, Artificial sequence; identical to SEQ ID NO: 197
  synthetic polynucleotide seqeuence

SEQ ID NO: 235 - protein, 20 aa, Artificial sequence
  synthetic polynucleotide seqeuence
TNTQTYREDLRTLLRYYNQS

SEQ ID NO: 236 - DNA, 60 bp, Artificial sequence
  synthetic polynucleotide seqeuence
caacacacagacttaccgagaggacctgcggaccctgctccgctactacaaccagagcga

SEQ ID NO: 237 - DNA, 60 bp, Artificial sequence
  synthetic polynucleotide seqeuence
caacacacagacttaccgagacaacctgcgcaccgcgctccgctactacaaccagagcga

SEQ ID NO: 238 - DNA, 59 bp, Artificial sequence
  synthetic polynucleotide seqeuence
cctgcggaacctgcgcggctactacaaccagagcgaggccaggtctcacatcatccaga

SEQ ID NO: 239 - DNA, 59 bp, Artificial sequence
  synthetic polynucleotide seqeuence
cctgcggaacctgcgcggctactacaaccagagcgaggccgggtctcacatcatccaga

SEQ ID NO: 240 - DNA, 58 bp, Artificial sequence
  synthetic polynucleotide seqeuence
ggaagagctcaggtggaaaaggagggagctgctctcaggctgcgtccagcaacagtgc

SEQ ID NO: 241 - DNA, 58 bp, Artificial sequence
  synthetic polynucleotide seqeuence
ggaagagctcaggtggaaaaggagggagctactctcaggctgcgtccagcaacagtgc

SEQ ID NO: 242 - DNA, 58 bp, Artificial sequence
  synthetic polynucleotide seqeuence
acagggcattttcttcccacaggtggaaaagcagggagctgctctcaggctgcgtgta

SEQ ID NO: 243 - DNA, 58 bp, Artificial sequence
  synthetic polynucleotide seqeuence
acagggcattttcttcccacaggtggaaaaggagggagctactctcaggctgcgtgta

SEQ ID NO: 244 - DNA, 33 bp, Artificial sequence
  synthetic polynucleotide seqeuence
gtggaaaaggagggagctactctcaggctgcgt

SEQ ID NO: 245 - DNA, 33 bp, Artificial sequence
  synthetic polynucleotide seqeuence
gtggaaaaggagggagctgctctcaggctgcgt

SEQ ID NO: 246 - DNA, 33 bp, Artificial sequence; identical to SEQ ID NO: 244
  synthetic polynucleotide seqeuence

SEQ ID NO: 247 - protein, 149 aa, Artificial sequence
  synthetic polynucleotide seqeuence
MAVMAPRTLLLLLSGALALTQTWAGSHSMRYFFTSVSRPGRGWPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIA

SEQ ID NO: 248 - DNA, 448 bp, Artificial sequence
  synthetic polynucleotide seqeuence
atggccgtcatggcgccccgaaccctcctcctgctactctcgggggccctggccctgacccagacctgggcgggctcccactccatgaggtatttcttcacatccgtgtcccggcccggccgcggggagccccgcttcatcgccgtgggctacgtggacgacacgcagttcgtgcggttcgacagcgacgccgcgagccagaagatggagccgcgggcgccgtggatagagcaggaggggccggagtattgggaccaggagacacggaatatgaaggcccactcacagactgaccgagcgaacctggggaccctgcgcggctactacaaccagagcgaggacggttctcacaccatccagataatgtatggctgcgacgtggggccggacgggcgcttcctccgcgggtaccggcaggacgcctacgacggcaaggattacatcgccc

SEQ ID NO: 249 - DNA, 448 bp, Artificial sequence
  synthetic polynucleotide seqeuence
atggccgtcatggcgccccgaaccctcgtcctgctactctcgggggctctggccctgacccagacctgggcgggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggttctcacaccgtccagaggatgtatggctgcgacgtggggtcggactggcgcttcctccgcgggtaccaccagtacgcctacgacggcaaggattacatcgccc

(19) United States
(12) Patent Application Publication
(10) Pub. No.: US 2015/0379195 A1
(43) Pub. Date: Dec. 31, 2015

(54) SOFTWARE HAPLOTYING OF HLA LOCI

(71) Applicant: The Board of Trustees of the Leland Stanford Junior University, Stanford, CA (US)

(72) Inventors: Chunlin Wang, Palo Alto, CA (US); Michael N. Mindrinos, Palo Alto, CA (US); Mark M. Davis, Atherton, CA (US); Ronald W. Davis, Stanford, CA (US); Sujatha Krishnakumar, Palo Alto, CA (US); Konstantinos Barsakis, Menlo Park, CA (US); Marcelo Anibal Fernandez-Vina, Stanford, CA (US)

(21) Appl. No.: 14/749,491

(22) Filed: Jun. 24, 2015

Related U.S. Application Data
(60) Provisional application No. 62/017,069, filed on Jun. 25, 2014.
(60) Provisional application No. 62/057,765, filed on Sep. 30, 2014.

(51) Int. Cl. G06F 19/22; C12Q 1/68

(52) U.S. Cl. CPC G06F 19/22; C12Q 1/6881; C12Q 2600/156

(57) ABSTRACT

Methods are provided to determine the genomic sequence of the alleles at the HLA gene. The resultant sequences provide linkage information between different exons, and produces the unique sequence at each gene from the two alleles of the individual sample being typed. The sequence information provides an accurate HLA haplotype. Methods to decrease allele dropout during long range PCR reactions are also disclosed.

### GOVERNMENT RIGHTS

This invention was made with Government support under contracts HG000205, AI090019, NS073581, AI090043, 27220100025C, MH096262 awarded by the National Institutes of Health and HDTRAI1-11-1-0058, WX81XWH-11-PRMRP-IIRA awarded by the Department of Defense. The Government has certain rights in this invention.

### BACKGROUND OF THE INVENTION

Software methods can increase the ability to accurately process large amounts of data. Polymorphic genes, such as the human leukocyte antigen (HLA) genes, have been traditionally difficult to sequence and characterize. For example, obtaining accurate, high-throughput results can be cost-prohibitive. Standard technologies present technical challenges when trying to accurately discriminate between highly related genes and their many alleles. For example, it has traditionally been difficult to accurately characterize both maternal and paternal alleles of a given HLA gene locus. Therefore, a need exists in the art for an accurate, high-resolution, and cost-effective methodology to type polymorphic and highly polymorphic genomic regions, such as the human HLA genes.

The HLA genes are among the most polymorphic in the human genome. They play a pivotal role in the immune response and have been implicated in numerous human pathologies, especially autoimmunity and infectious diseases. Despite their importance, however, they are rarely characterized comprehensively because of the prohibitive cost of standard technologies and the technical challenges of accurately discriminating between these highly related genes and their many alleles. Methodologies to type HLA genes can be used in the clinical setting, e.g. to test histocompatibility in transplantation, in disease-association studies, and for diagnostic testing.

The human leukocyte antigen (HLA) system can refer to the locus of genes that encode for the major histocompatibility complex (MHC). In humans, the MHC region, where HLA genes are located, spans approximately 4 million base pairs on the short arm of chromosome 6. It can be divided into 3 separate regions referred to as class I, class II and class III. The class I region includes the class I HLA genes designated HLA-A, HLA-B, and HLA-C. In addition are the nonclassical class I HLA genes: HLA-E, HLA-F, HLA-G, HLA-H, HLA-J, HLA-X, and MIC. The class II region contains the HLA-DP, HLA-DQ and HLA-DR loci, which encode the α and β chains of the classical class II HLA molecules designated HLA-DP, DQ and DR. Nonclassical genes designated DM, DN and DO have also been identified within class II. The class III region contains a heterogeneous collection of more than 36 genes. The loci constituting the HLA genes are highly polymorphic. Several thousand different allelic variants of class I and class II HLA molecules have been identified in humans.

Driven by selection of alleles for protection against environmental insult and infection, HLA genes have extensive degree of polymorphism, which enables immune system to fight with a high variety range of pathogens. The specific protein sequences of the highly polymorphic HLA locus play a major role in determining histocompatibility of transplants, as well as important insight into susceptibility of a number of immune related disorders, including celiac disease, rheumatoid arthritis, insulin-dependent diabetes mellitus (i.e. type I diabetes), multiple sclerosis, narcolepsy and the like. Matching of donor and recipient HLA genes (e.g. HLA-A, -B, -C, -DQB1, -DPB1, and -DRB1) prior to allogeneic transplantation can influence allograft survival. Therefore, HLA matching can be required as a clinical prerequisite before transplantation of tissue (e.g. renal, bone marrow, cord blood, kidney, liver, and the like).

Conventionally, transplantation matching has been performed by serological and/or cellular typing. However, serological typing can be frequently problematic, due to the availability and cross-reactivity of alloantisera and because live cells are required. A high degree of error and variability is also inherent in serological typing. Therefore, DNA typing can be preferable to serological tests.

In some methods, polymerase chain reaction (PCR) amplified products are hybridized with sequence-specific oligonucleotide probes (PCR-SSO) to distinguish between HLA alleles. This method requires a PCR product of the HLA gene of interest be produced and then dotted onto nitrocellulose membranes or strips. Then each membrane is hybridized with a sequence specific probe, washed, and then analyzed by exposure to x-ray film or by colorimetric assay depending on the method of detection.

More recently, a molecular typing method using sequence specific primer amplification (PCR-SSP) has been described. In PCR-SSP, allelic sequence specific primers amplify only the complementary template allele, allowing genetic variability to be detected with a high degree of resolution. This method allows determination of HLA type simply by whether or not amplification products are present or absent following PCR. In PCR-SSP, detection of the amplification products may be done by agarose gel electrophoresis.

Currently, direct DNA sequencing or “sequence based typing” (SBT) can provides a higher resolution test. Determining the genomic sequence can be used to discriminate alleles at the nucleotide level, where minor differences in sequence have great impact on the phenotype of the HLA genes. However, HLA genes span large regions (e.g. between 5 Kb to 15 Kb) in the human genome. Current DNA sequencing approaches target one or a few of disjoined exons in the genomic DNA. Further, since each individual is diploid, it is important to characterize the unique sequence from each gene to understand how these changes are reflected at the protein level. Without linkage information between those exons, the fragmental information from individual exons generates incomplete data and is not sufficient for definitive haplotype determination.

In addition, the high genetic polymorphism of HLA presents a challenge to the next generation sequencing (NGS) technology used in HLA typing. The NGS involves PCR amplification of specific genomic regions of HLA genes and sequencing of these amplicons. While NGS permits the highest resolution at a single nucleotide level between different genotypes, one of its limitations is the preferential amplification of one allele (i.e. allele dropout) in a heterozygous sample. In other words, long range PCR amplification can unequally amplify maternally and paternally inherited HLA genes. As a result, allele dropout may result in incorrect genotyping, such as false homozygosity, or misdetection of mutations. Allele dropout may arise from differences in the GC content between alleles, differences in allele size, mis-matches between primer and template DNA resulting from single nucleotide polymorphisms (SNPs) in the primer-binding site, low amounts or poor quality of DNA, and/or inappropriate PCR conditions. Traditionally, it has been difficult to generate accurate sequence data of both alleles. This has made it difficult to call both alleles for targeting HLA genes.

Improved methods of typing polymorphic genes, such as HLA, are of great interest for research and clinical applications. Prevention of allele dropout during PCR is beneficial to the reliability of typing polymorphic genes, such as HLA. Methods of this disclosure describe a novel method not only to generate accurate sequence data for diploid and polymorphic targeted HLA genes, but also a machine readable code able to determine the HLA genotype accurately by implementing a novel algorithm into a software to process the sequence data. The software processing, data processing and other methods described herein can be employed to type polymorphic genes.

### SUMMARY OF THE INVENTION

Software and data-processing methods are provided for accurately determining the sequence of a polymorphic genomic region (e.g. HLA). Compositions, including sets of primers for amplification, and methods are provided for accurately determining the sequence of a highly polymorphic regions (e.g. determining the HLA genotype of an individual), or for simultaneous determination of HLA genotypes from a plurality of individuals simultaneously. In some embodiments the HLA genotype comprises sequences of one or more HLA Class I genes. In other embodiments the HLA genotype comprises sequences of one or more HLA Class II genes. In some embodiments, the genotype of all major HLA genes including HLA-A, HLA-B, HLA-C, HLA-DPA1, HLA-DPB1, HLA-DQA1, HLA-DQB1, HLA-DRB1, HLA-DRB3, HLA-DRB4, HLA-DRB5 are determined. In other embodiments, the genotype of a combination of HLA-A, HLA-B, HLA-C and HLA-DRB1 are tested. The information provided by the methods of analysis is useful in screening individuals for transplantation, as well as for the determination of HLA genotypes associated with various diseases, including a number of immune-associated diseases.

The methods of the invention comprise the steps of amplifying multiple exons and intervening introns of an HLA gene in a long-range PCR reaction using a mixture of regular dNTPs and dNTP analogs; deep sequencing the amplified gene; and performing deconvolution analysis to resolve the genotype of each allele. The methods of the invention make an accurate genotype calling with a novel mapping-filtering-enumerating-counting algorithm. The methods of the invention can generate an accurate consensus sequence, which can be used to verify genotype results. The methods of the invention thus call HLA genotype accurately by mapping-filtering-enumerating-counting algorithm; afterwards determine the genomic sequence of a particular HLA gene, including both intron and exons. The resultant consensus sequence can be used to prove the accuracy of genotype results. The resultant consensus sequence from each of the analyzed loci provides linkage information between different exons, and is used to produce the unique sequence from each allele of the gene. The sequence information in intron regions, along with the exon sequences provides an accurate HLA genotype, which can be critical to solve phasing problems that current HLA haplotyping approaches have thus far failed to address.

In some embodiments, each HLA gene being analyzed is amplified from genomic DNA in a single long-range polymerase chain reaction spanning the majority of the coding regions and covering most known polymorphic sites. The benefits of this approach are that (a) more polymorphic sites are sequenced to provide genotyping information of higher definition and the physical linkage between exons can be determined to resolve combination ambiguity; (b) long-range PCR primers can be placed in less polymorphic regions, minimizing primer filtering by polymeric sites, therefore allowing for improved resolution of genetic differences; and (c) exons of the same gene can be amplified in one fragment, thereby decreasing coverage variability.

In the amplification step, a preferred method is long range polymerase chain reaction. For each HLA gene, a plurality of gene specific PCR primers are designed to amplify a genomic area covering multiple exons and intervening introns of the HLA gene of interest in a single reaction. Generally at least 3 exons are amplified, or at least 4 exons are amplified, or more exons, up to the entire gene, are amplified. For example, for Class I genes, e.g. HLA-A, -B, and -C, primers may be selected to amplify the first seven exons of each gene.

In some embodiments, a mixture of regular dNTPs and a dNTP analog with a predetermined ratio is used in the long range PCR reaction. The dNTP analog used includes, but is not limited to, 5-aminoallyl-2′-dCTP, 5-(3-aminoallyl)-2′-deoxycytidine-5′-triphosphate (5-aminoallyl-2′-dCTP), 2′-deoxycytidine-5′-O-(1-thiotriphosphate) ((1-thio)-2′-dCTP), 2′-deoxy-5-methylcytidine 5′-triphosphate (5-methyl-2′-dCTP), 2-thio-2′-deoxycytidine-5′-triphosphate (2-thio-2′-dCTP), 5-iodo-2′-deoxycytidine-5′-triphosphate (5-iodo-2′-dCTP), 2-amino-2′-deoxyadenosine-5′-triphosphate (2-amino-2′-dATP), 2-thiothymidine-5′-triphosphate (thio-TTP), 5-propynyl-2′-deoxycytidine-5′-triphosphate (5-propynyl-2′-dCTP), N⁴-methyl-2′-deoxycytidine-5′-triphosphate (N⁴-methyl-2′-dCTP), 7-deaza-2′-deoxyadenosine-5′-triphosphate (7-deaza-2′-dATP), 2′-deoxyguanosine-5′-O-(1-thiotriphosphate) ((1-thio)-2′-dGTP), 2′-deoxyadenosine-5′-O-(1-thiotriphosphate) ((1-thio)-2′-dATP), 5-bromo-2′-deoxycytidine-5′-triphosphate (5-bromo-2′-dCTP), and 7-deaza-2′-deoxyguanosine-5′-triphosphate (7-deaza-dGTP). The ratio between the dNTP analog and the corresponding dNTP may be chosen from about 1:3, about 1:2, about 1:1, about 2:1, and about 3:1. In one embodiment, a ratio is about 3:1. In one embodiment, a ratio is about 2.7:1. In one embodiment, a ration is about 3.3:1. At least one dNTP analog is used together with regular dNTPs in the long range PCR reaction. In another embodiment, a preferred list of dNTP analogs is (1-thio)-2′-dCTP, N⁴-methyl-2′-dCTP, 7-deaza-2′-dATP, (1-thio)-2′dGTP, and 7-deaza-dGTP. In still another embodiment, a preferred list of dNTP analogs is N⁴-methyl-2′-dCTP and 7-deaza-dGTP.

In one embodiment, the polymerase used in the long range PCR includes, but is not limited to, Crimson LongAmp® Taq DNA Polymerase and Phire Hot Start II DNA Polymerase. In another embodiment, a preferred polymerase is Crimson LongAmp® Taq DNA Polymerase.

Genes in the same HLA locus share a high degree of sequence similarity to each other and to pseudogenes, or to other HLA genes (e.g., HLA-B, and HLA-C genes are similar to each other), which similarity is challenging for the specific amplification of a desired gene target. Gene-specific primers are selected from the regions flanking the gene target. Exemplary primers are provided herein for this purpose. Generally a PCR amplification is performed, where each target is amplified with one or more primers. In some embodiments, nested PCR is performed (e.g. with at least two sets of primers, one set internal to the other). The most polymorphic exons and the intervening sequences for each gene are amplified as a single product. The primers are chosen to lie outside of regions of high variability, and if necessary multiple primers are included in a reaction, to ensure amplification of all known alleles for each gene.

In some embodiments, at least one gene-specific primer comprises at least one dNTP analog. In some embodiments at least one gene-specific primer comprises at least one nucleoside linkage that increases resistance to nuclease digestion (e.g. a phosphothioate linkage). Some primers comprise both phosphodiester and phosphothioate linkages (e.g. the tables of primers listed herein use an * symbol to mark candidate regions for a phosphothioate linkage).

In some embodiments, primers are designed to hybridize regions that lie outside of regions of high variability, and if necessary multiple primers are included in a reaction, to ensure amplification of all known alleles for each gene. In some embodiments, the ratio of primer concentration can range from about 1:1 to about 1:10.

Following amplification, the concentration of the amplicons can be determined. In some embodiments, an approximate equimolar quantity of each locus is pooled (e.g. to create reaction conditions with equal representation of each gene). In some embodiments amplicons are ligated. In other embodiments, a non-equimolar quantity of amplicons are used.

Amplicons can be randomly sheared to an average fragment size of from about 200 to about 700, usually from about 300 to about 600 bp, or from about 400 to about 500 bp in length. In preparation for sequencing, barcodes can be ligated to the resulting fragments, where each barcode includes a target specific identifier for the source of the genomic DNA and the gene; and a sequencing adaptor. Sequence length in some embodiments can range from about 100 to about 500 nucleotides. Sequencing can be be performed from each end of the fragment. Each sequence can therefore be assigned to the sample and the gene from which it was obtained.

In other embodiments, after a long-range PCR amplification, the concentration of each amplicon is measured and an equimolar quantity of each amplicon is pooled to maximize the output of the ensuing multiplex process for DNA sequencing. In some embodiments, the ratios among amplicons are analyzed and determined by a computer device to balance amplicons before the sequencing step of HLA typing.

A report may be prepared disclosing the identification of the haplotypes of the alleles that are sequenced by the methods of the invention, and may be provided to the individual from whom the sample is obtained, or to a suitable medical professional.

In some embodiments, a kit is provided comprising a set of primers suitable for amplification of the one or more genes of the HLA locus, e.g. the class I genes: HLA-A, HLA-B, HLA-C; the Class II gene DRB, etc. The primers may be designed. Exemplary primers are listed as SEQ ID NO: 1-194 (e.g. Table 1; FIG. 64 and FIG. 65). In some embodiments a master mix of primers may be used. One exemplary master mix is comprised of the primers described in FIG. 64 (e.g. SEQ ID NO: 69 through SEQ ID NO: 111). The kit may further comprise a long range polymerase. The kit may further comprise regular dNTPs and at least one dNTP analog. The kit may further comprise reagents for amplification and sequencing. The kit may further comprise instructions for use; and optionally includes software for genotype calling.

Compositions, including sets of primers for amplification, and methods are provided for accurately determining one or more genotypes of an organism or for simultaneous determination of one or more genotypes from a plurality of organisms simultaneously. In some embodiments, the genomic region may be large. In some embodiments, a region to genotype may be a polymorphic genomic region or a highly polymorphic genomic region (e.g. HLA genomic region). In some embodiments, determining the genotype of a large genomic region may comprise amplifying a large nucleic acid by PCR to generate a long amplified nucleic acid (e.g. a large DNA molecule can be amplified using long-range PCR), fragmenting the amplified nucleic acid, and sequencing. In some embodiments, the sequencing is done with an excess of independent paired-end reads. In some embodiments, the sequencing generates data which can be analyzed using a computing device.

### BRIEF DESCRIPTION OF THE DRAWINGS

FIG. 1. Location of long-range PCR primers and PCR amplicons in HLA genes. (A) For class I HLA gene (HLA-A, -B, and -C), the forward primer is located in exon 1 near the first codon and the reverse primer is located in exon 7. For HLA-DRB1, the forward primer is located at the boundary between intron 1 and exon 2 and the reverse primer is located within exon 5. Note that the size of exon or intron in the drawing is not proportional to their actual size. (B) Agarose gel (0.8%) showing amplicons from long range PCR. HLA-A, -B, -C amplicons are 2.7 kb in length, and -DRB1 amplicon is around 4.1 kb.

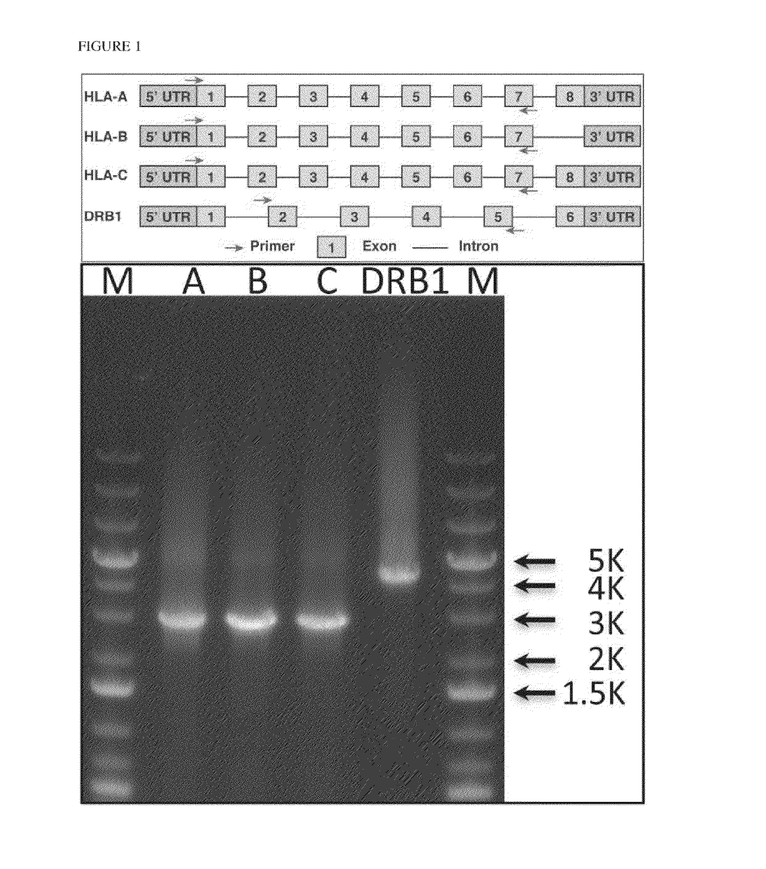

FIG. 2. Mapping patterns of sequencing reads on correct and incorrect references. (A) Central reads of an anchor point are defined as mapped reads, where the ratio between the length of the left arm and that of the right arm related to a particular point is between 0.5 and 2 as those are highlighted in red. (B) Mapping pattern of sequencing reads onto correct references (A and B) and onto an incorrect reference (C). (C) Alignment of references A, B, and C around the anchor point shown in (B). The anchor points are marked as two double-arrow line.

FIG. 3. Identification and verification of three novel alleles with insertions and deletions. (1.a) shows the coverage of overall reads (red) and central reads (blue) mapped onto HLA-A*02:01:01:01 cDNA reference in one clinical sample. (1.b) shows the partial alignment between a contig derived from reads mapped onto HLA-A*02:01:01:01 reference and HLA-A*02:01:01:01 reference. (1.c) shows the chromatogram of Sanger sequence on a clone derived from HLA-A PCR product from the same sample. Black arrows 1 highlight a 5-base ‘TGGAC’ insertion in coverage plot (1.a), alignment (1.b) and chromatogram (1.c). (2.a) shows the coverage of overall reads (red) and central reads (blue) mapped onto HLA-B*40:02:01 cDNA reference in one clinical sample. (2.b) shows the partial alignment between a contig derived from reads mapped onto HLAB* 40:02:01 reference and HLA-B*40:02:01 reference. (2.c) shows the chromatogram of Sanger sequence on a clone derived from HLA-B PCR product from the same sample. Black arrows 2 highlight an 8-base ‘TTACCGAG’ insertion in coverage plot (2.a), alignment (2.b) and chromatogram (2.c). (3.a) shows the coverage of overall reads (red) and central reads (blue) mapped onto HLA-B*51:01:01 genomic reference in one clinical sample. (3.b) shows the partial alignment between a contig derived from reads mapped onto HLA-B*51:01:01 reference and HLA-B*51:01:01 reference. (3.c) shows the chromatogram of Sanger sequence on a clone derived from HLA-B PCR product from the same sample. Black arrows 3 highlight a single base ‘A’ deletion in coverage plot (3.a), alignment (3.b) and chromatogram (3.c). In the coverage plots, exon regions are indicated with Roman numerals.

FIG. 4. Comparison of allele resolution (left) and combination resolution (right) if different regions of HLA genes were sequenced. Analysis was based on the IMGT/HLA reference sequence database released on Oct. 10, 2011. The allele resolution is defined as the percentage of alleles that can be resolved definitively when particular regions of a gene are analyzed. The combination resolution is defined as the percentage of combinations of two heterozygous alleles that can be resolved definitively when particular regions of a gene are analyzed. Note that due to the lack of sequence information outside exon 2 for the HLA-DRB1 gene, where only 15% reference sequences cover exon 3 and 7% reference sequences cover exon 4 region for the HLADRB1 gene, the difference between our method and conventional SBT methods over this gene can be estimated accurately.

FIG. 5. Sanger sequencing validation of the HLA-DRB1 genotype of the cell-line FH11 (IHW09385). (A) Coverage plots for the reference allele HLA-DRB1*11:01:02 (blue) and the predicted allele HLA-DRB1*11:01:01 (red) where the black triangle points to the difference in the coverage plots of these two alleles. (B) Partial Sanger sequencing chromatogram of the amplification products in the exon 2 region of HLA-DRB1 locus. (C) Alignment of HLA-DRB1*01:01:01, HLA-DRB1*11:01:01, and HLADRB1* 11:01:02 where the differences among the three alleles are highlighted in red and the intron-exon boundary is indicated in green. (D) Partial Sanger sequencing chromatogram of the amplification products in the intron 2 region of HLA-DRB1 locus. Arrows link positions that are different between the three references in the alignment, and the corresponding positions in the chromatograms. The IMGT-HLA database reports that the HLA-DRB1 locus of FH11 is heterozygous for 01:01:01/11:01:02. Our Illumina data suggest that it should be heterozygous for 01:01:01/11:01:01. The chromatograms in (B) and (D) match the expected pattern of mixture of HLA-DRB1*01:01:01/11:01:01, instead of HLA-DRB1*01:01:01/11:01:02.

FIG. 6. Sanger sequencing validation of the genotype of HLA-B locus of the cell-line FH34 (IHW09415). A) Coverage plots for the reference allele HLA-B*15:35 (yellow line) and the predicted allele HLA-B*15:21 (black dash line). Note the there is no reference sequence for the HLA-B*15:35 allele in exon 1 region, which is the reason for zero coverage in this region (highlighted by the black triangle). There is no reference sequence for the HLA-B*15:35 allele in exon 5, 6, 7 either. Although HLA-B*15:21 and HLA-B*15:35 are identical in exon 4, HLA-B*15:35 has lower coverage than HLAB* 15:21 (highlighted in gray triangle) due to removal of reads that did not pass the pair end filter. B) Alignment of HLA-B*15:35 and HLA-B*15:21 in partial exon 2 and 3 regions where the differences among the three alleles are highlighted in red and the intron-exon boundary is indicated in green. C) Partial Sanger sequencing chromatogram of the amplification products in the exon 2 region of HLA-B locus. The arrows point out the chromatogram pattern matching the expected pattern of mixture of HLA-B*15:21 and HLA-B*15:35. The reference alleles listed for HLA-B locus of FH34 is 15/15:21 and based on our sequencing data we are able to extend the resolution to 15:21/15:35

FIG. 7. Sanger sequencing validation of the HLA-B genotype of the cell-line ISH3 (IHW09369). (A) Coverage plots for reference HLA-B*15:26N (red) and HLA-B*15:01 (blue). Reads align continuously onto exons 2, 3, 4, and 5, but not exon 1 of HLAB* 15:26N. There are reads aligning to exon 1 of HLA-B*15:01 (black triangle). (B) Partial Sanger sequencing chromatogram of the amplification products in the exon 1 region of HLA-B locus. The nucleotide in the 11th position of exon 1 is C as in HLAB* 15:01:01. (C) Alignment of HLA-B*15:01:01:01 and HLA-B*15:26N where the differences among the three alleles are highlighted in red and the intron-exon boundary is indicated in green. (D) Partial Sanger sequencing chromatogram of the amplification products in the exon 3 region of HLA-B locus. Arrows link positions that are different between the three references in the sequence alignment and the corresponding position in the chromatograms. The IHWG cell-line database reports that the HLA-B locus of ISH3 is homozygous for 15:26N. The chromatograms in panes (B) and (D) suggest that this is a new allele with exon 1 sequence as that of HLA-B*15:01:01:01 and exons 2, 3, 4, and 5 sequence as that of HLA-B*15:26N. 101 102 103 104 105 0 50 100 150 200 250 300 350 400 450 Minimum Coverage Allele

FIG. 8. Minimum coverage (sorted ascending) of all HLA alleles in 59 clinical samples. Only three alleles were typed with minimum coverage less than 100.

FIG. 9. Schematic diagram of primer selection criteria. 500 bp region was set at both ends of each HLA gene as a cushion region. Primers are chosen from 1500 bp region upstream of forward cushion region and 1500 bp region downstream of the reverse cushion region. Each primer is systematically examined for conservation and specificity. Only those with highest conservation and specificity index (CSI) are picked up.

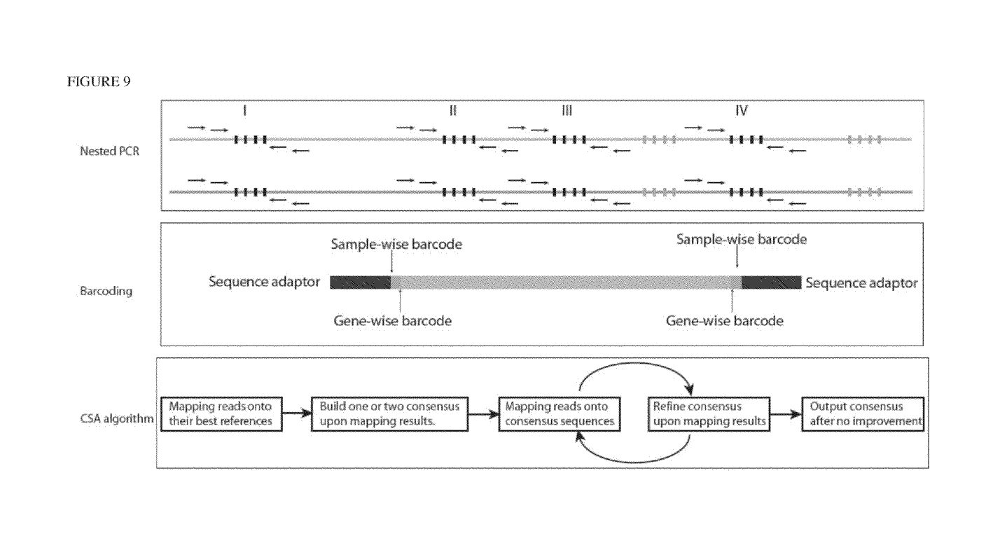

FIG. 10 is a schematic of the HLA locus conservation and specificity.

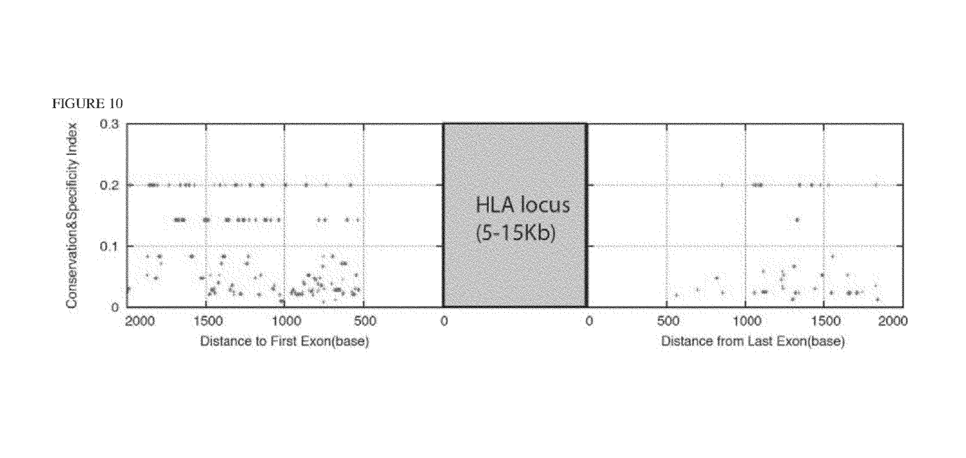

FIG. 11 is a schematic of the chromatid sequence alignment.

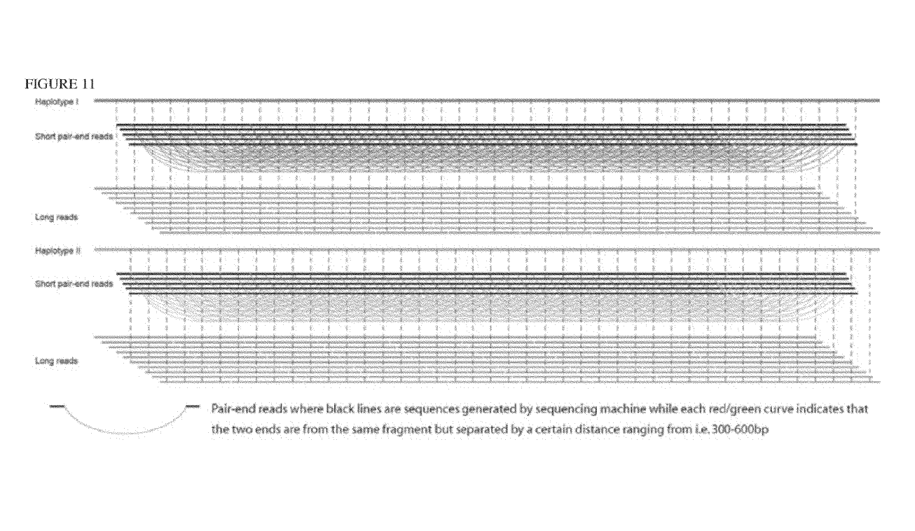

FIG. 12 is a flowchart depicting an exemplary sequence of steps which may be practiced in accordance with a method of the present disclosure.

FIG. 13 is a table depicting some exemplary data using different dNTP analogs in a polymerase chain reaction.

FIG. 14 is a table depicting some exemplary results of using five different dNTP analogs among nine samples.

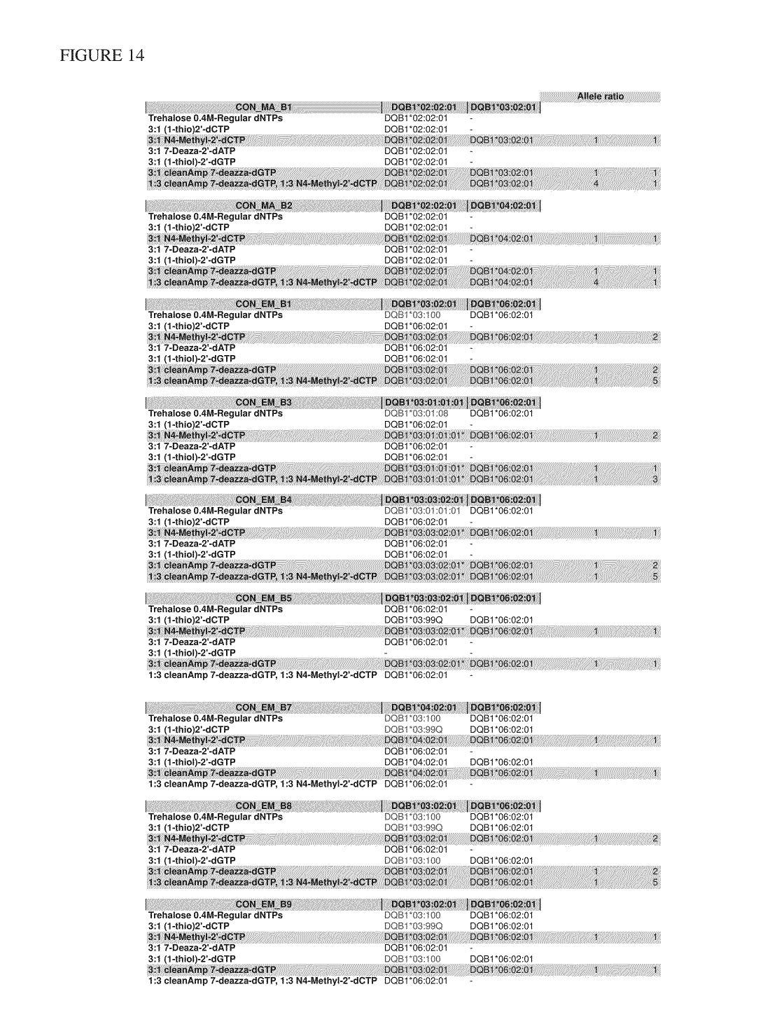

FIG. 15 is a table depicting exemplary results demonstrating the final error rate percentage using different next generation sequencing platforms.

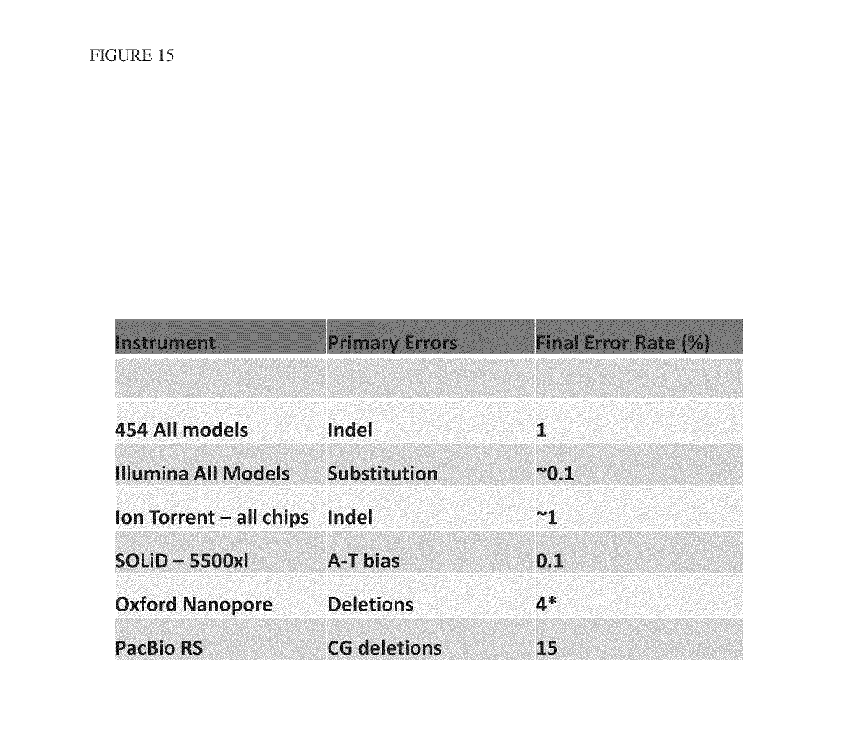

FIG. 16 depicts an illustration comparing exon-wise amplification of a few exons versus whole-gene amplification

FIG. 17 depicts an illustration of an exemplary method to design an assay.

FIG. 18 depicts exemplary results comparing the ability of different enzymes to amplify HLA-B.

FIG. 19 depicts exemplary results comparing the ability of different enzymes to amplify HLA-A.

FIG. 20 depicts exemplary results when an enhancer is added to a reaction.

FIG. 21 depicts exemplary results when different enhancers are added to a reaction.

FIG. 22 depicts exemplary results when trehelose is added to a reaction.

FIG. 23 depicts an exemplary process workflow.

FIG. 24 depicts exemplary results demonstrating coverage variance among different HLA genes.

FIG. 25 depicts exemplary results demonstrating reproducibility of coverage.

FIG. 26 depicts exemplary polymorphic nucleotide positions of two hybrid alleles.

FIG. 27 depicts exemplary ambiguities such as exon shuffling, segmental exchange, and substitutions in untested segments.

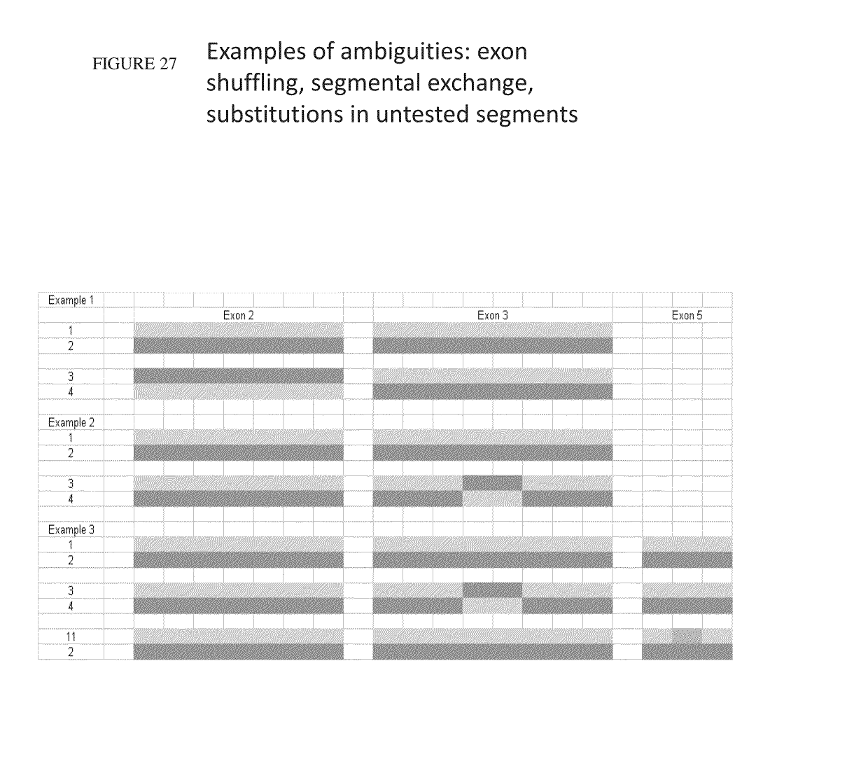

FIG. 28 depicts exemplary exonic substitutions.

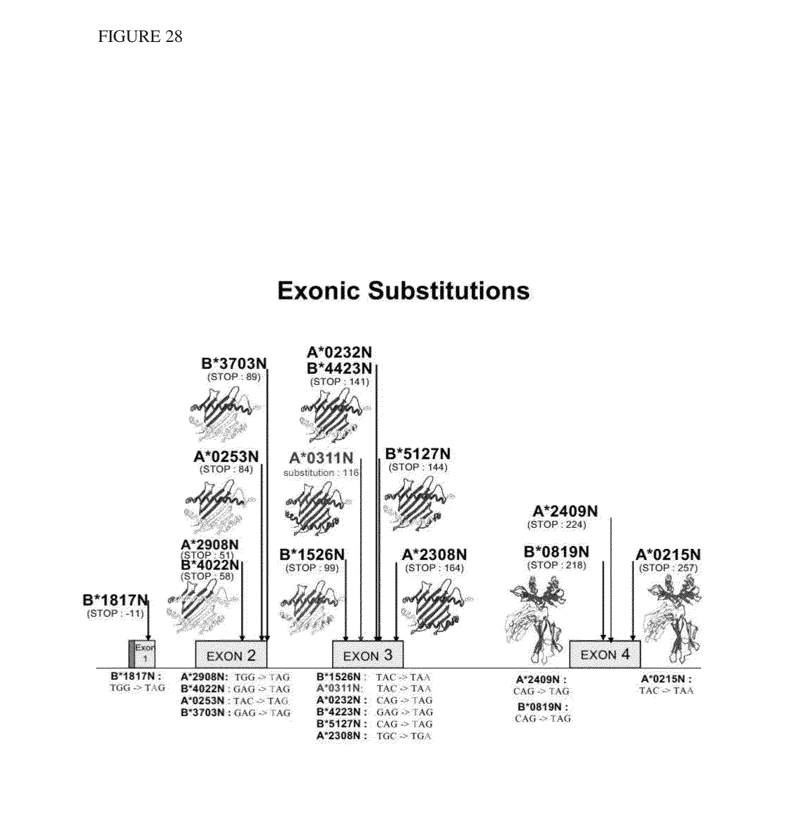

FIG. 29 depicts an illustration of exemplary implications for HLA-A antigen mismatches between patients and donors.

FIG. 30 depicts an exemplary HLA-A allele groups with an extra C′ insertion.

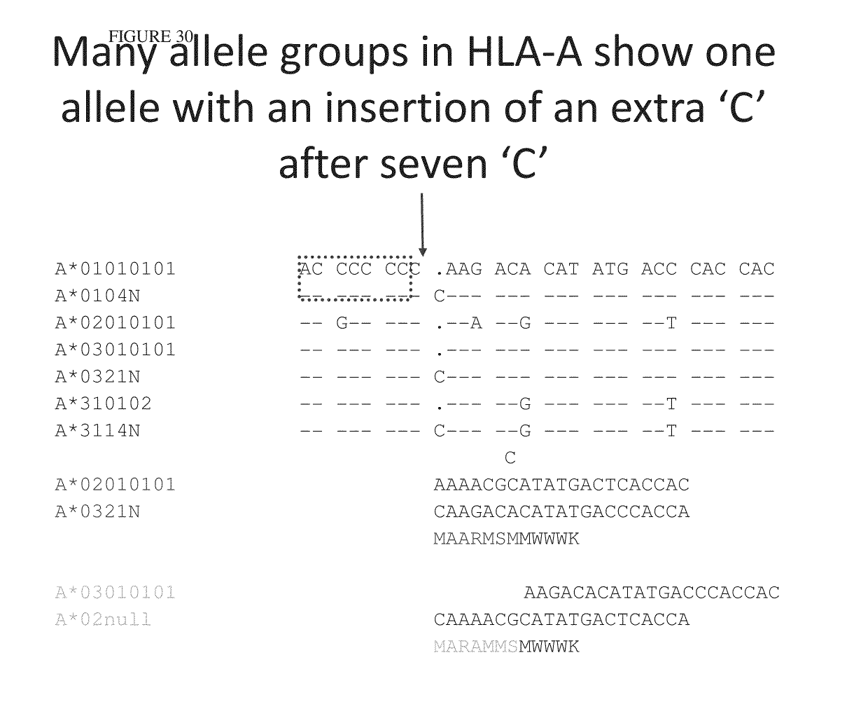

FIG. 31 depicts exemplary results and resolutions of common, well documented, and clinically relevant null-alleles.

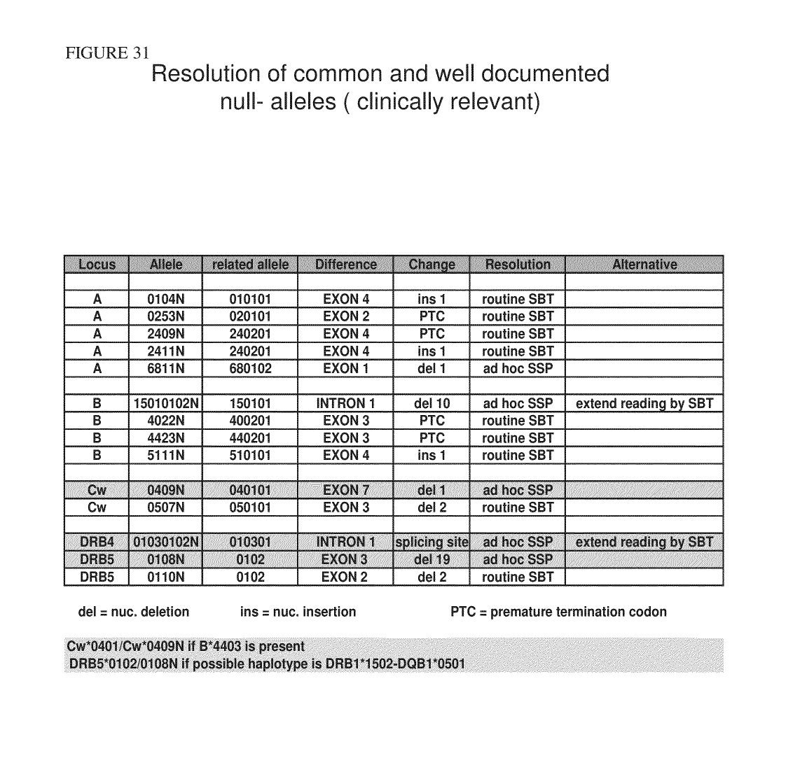

FIG. 32 depicts exemplary results of allele detection and coverage.

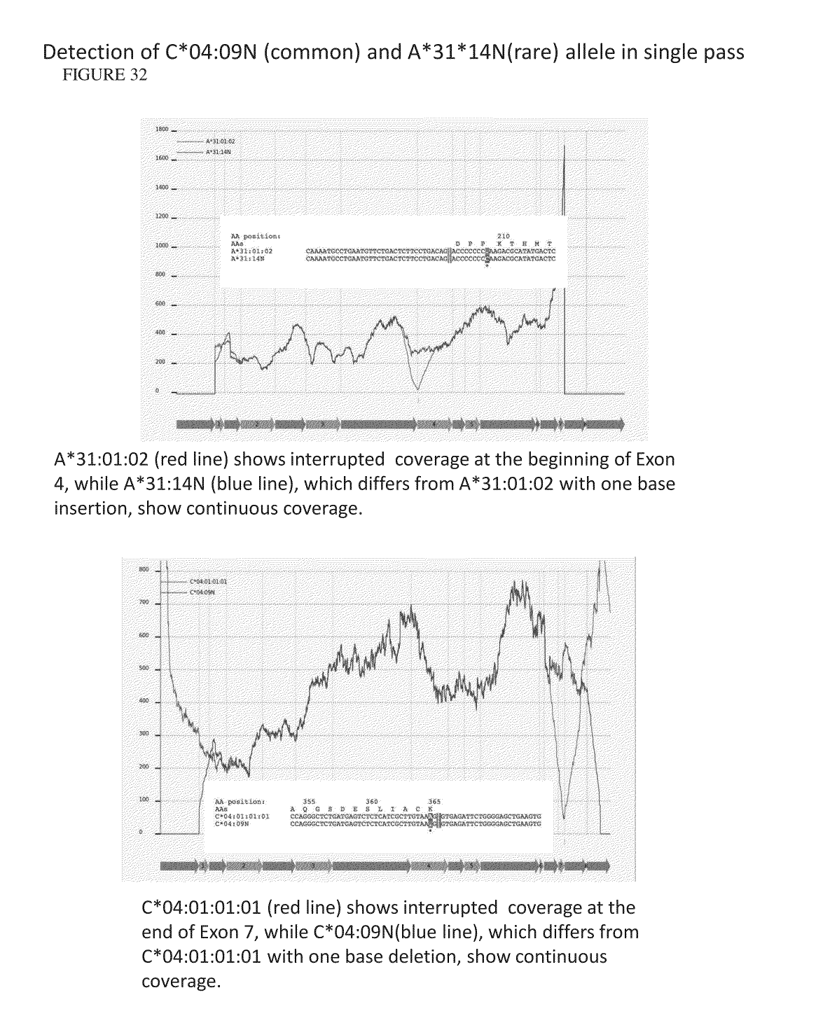

FIG. 33 depicts exemplary genotype differences.

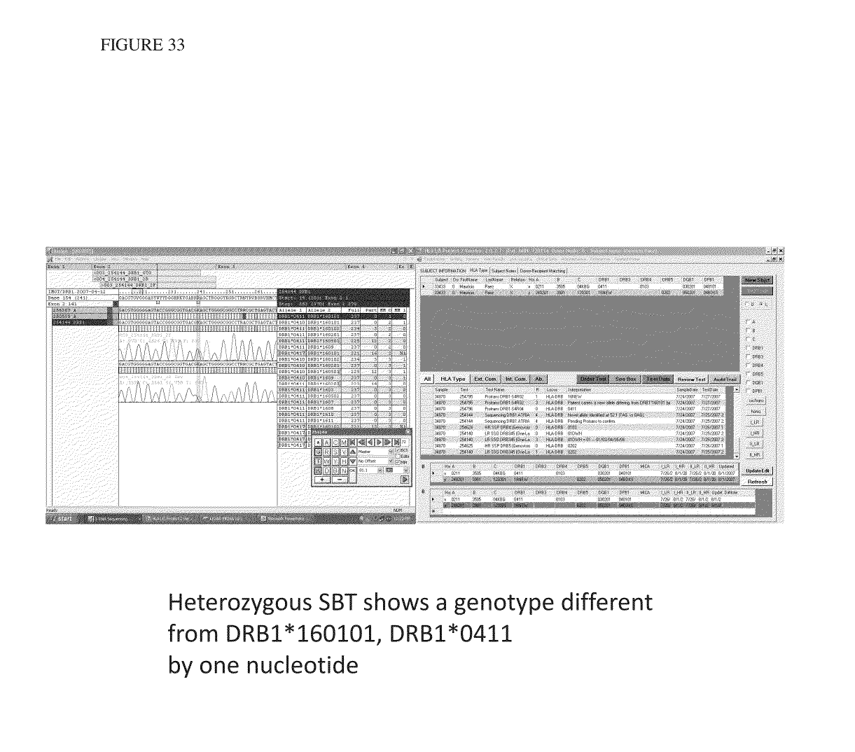

FIG. 34 depicts exemplary group specific amplification.

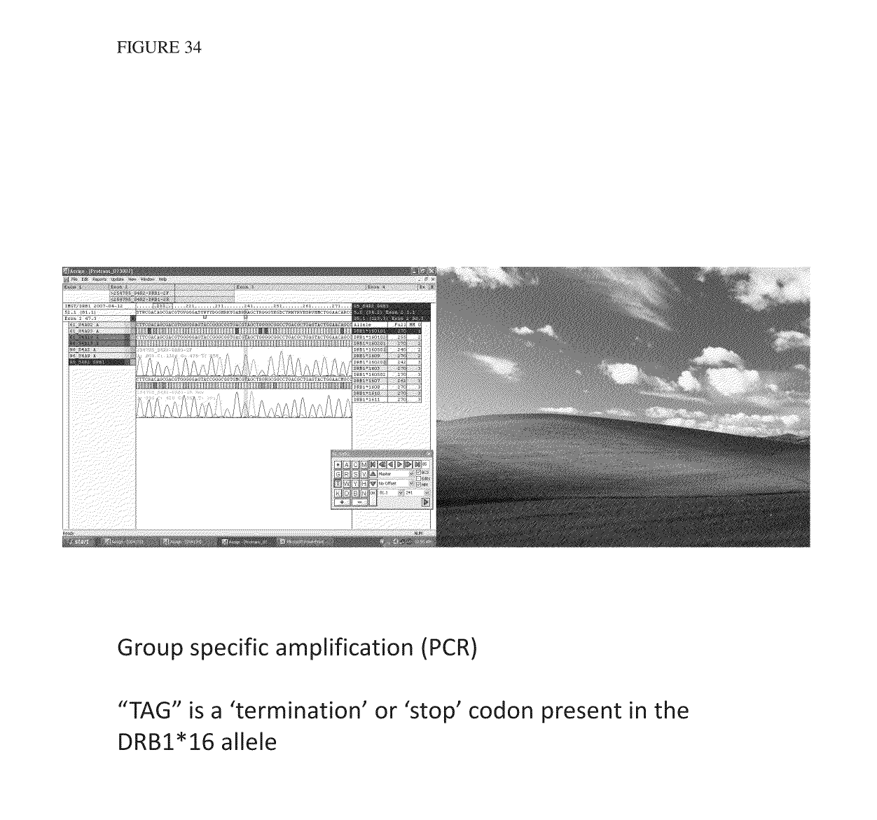

FIG. 35 depicts exemplary Q alleles and biological relevance.

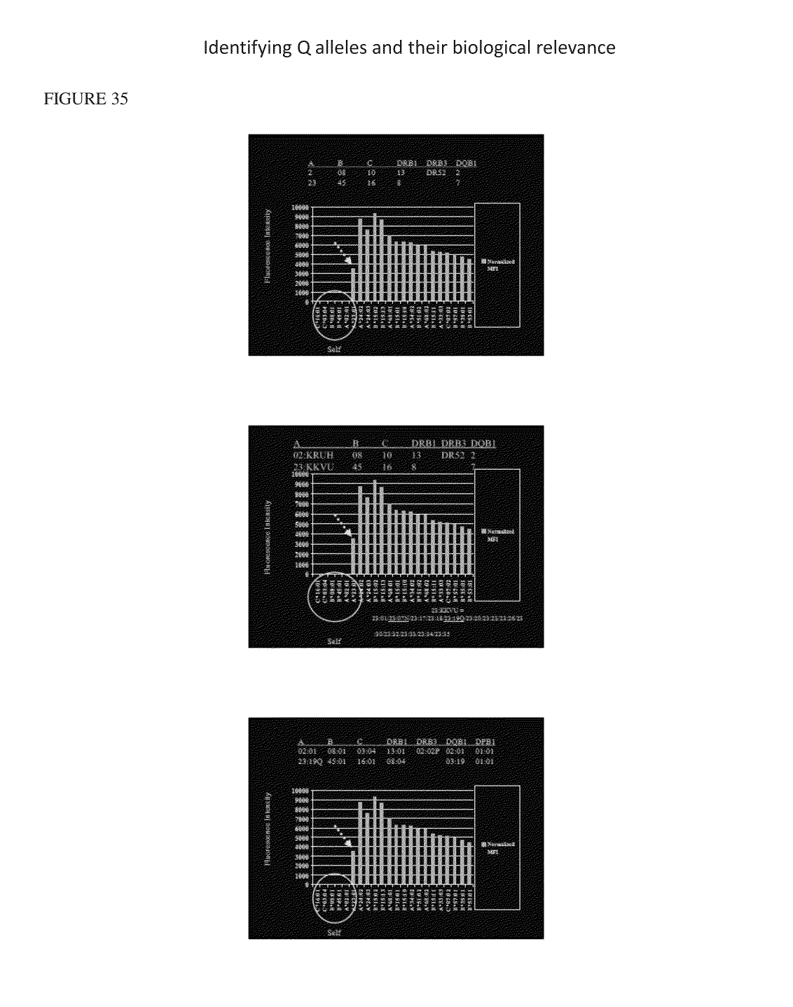

FIG. 36 depicts exemplary nucleotide replacement at the splicing site.

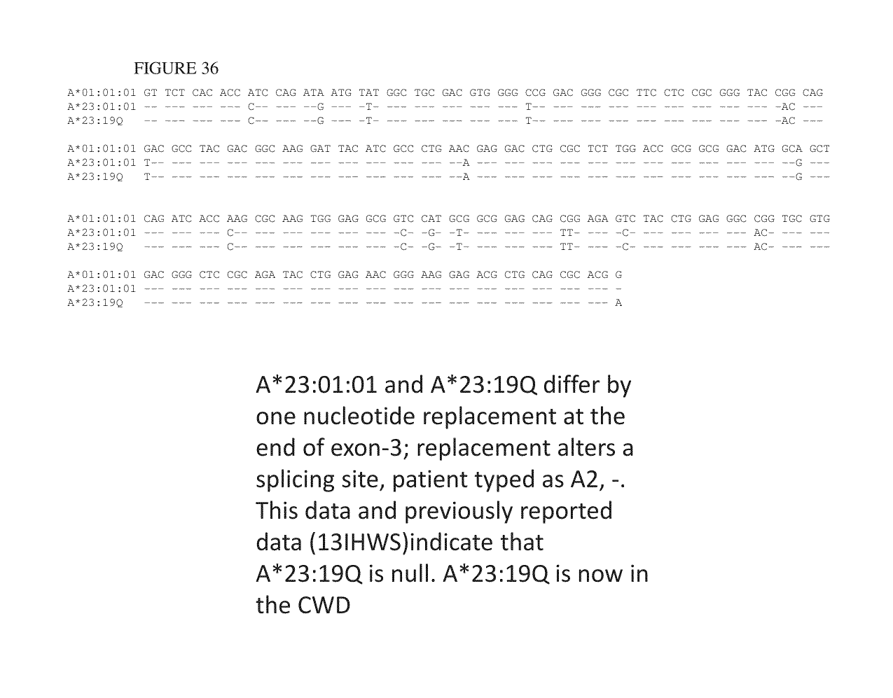

FIG. 37 depicts exemplary new findings obtained through NGS application.

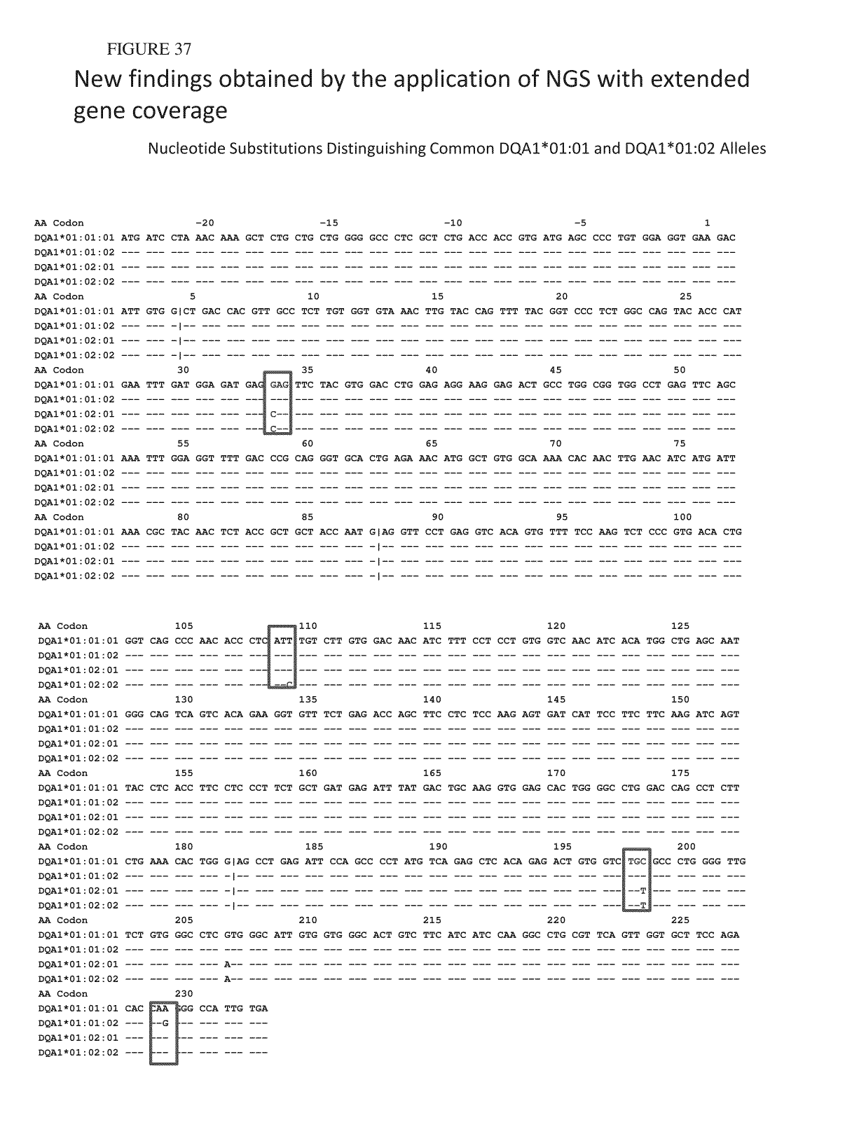

FIG. 38 depicts exemplary results of gene coverage.

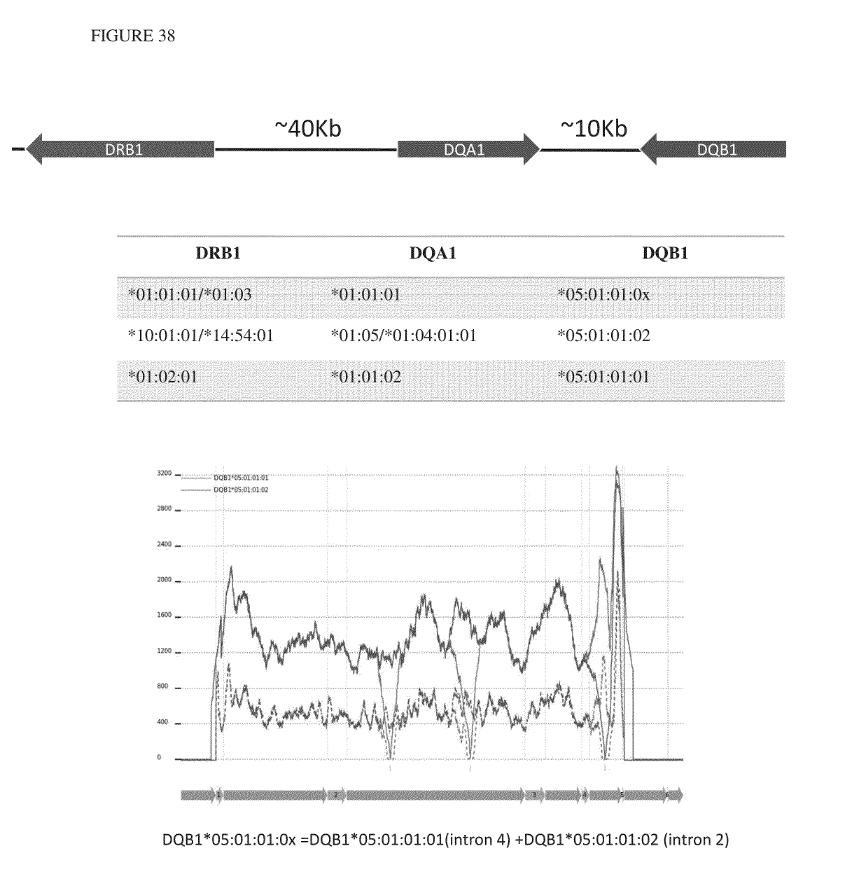

FIG. 39 depicts exemplary results of silent mutations leading to haplotype diversity.

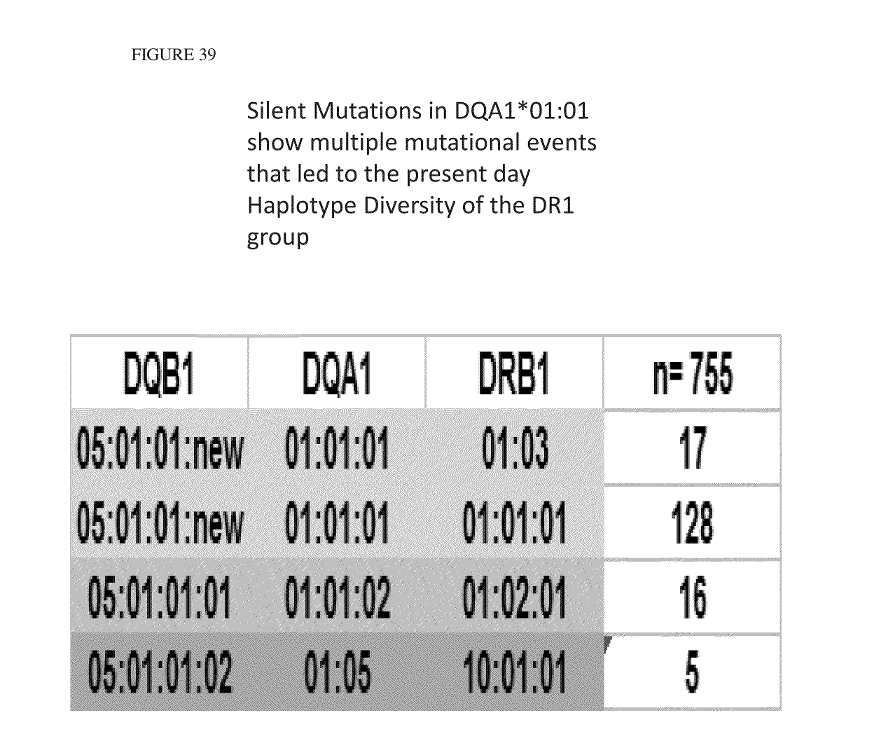

FIG. 40 depicts exemplary results of nucleotide substitutions generating allelic diversity.

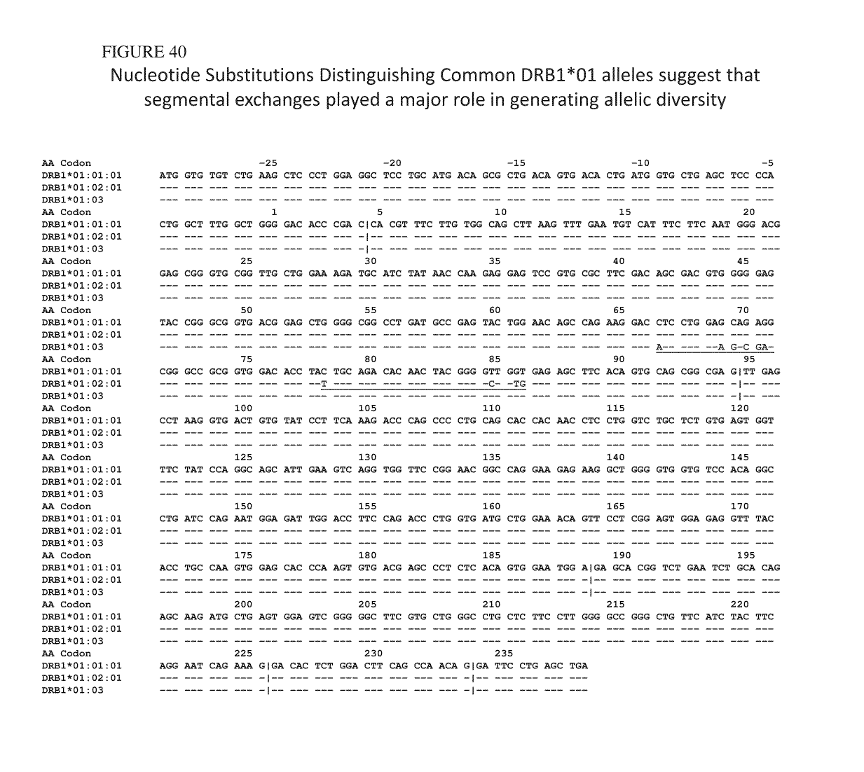

FIG. 41 depicts exemplary silent mutations showing unexpected complexity in haplotype evolution.

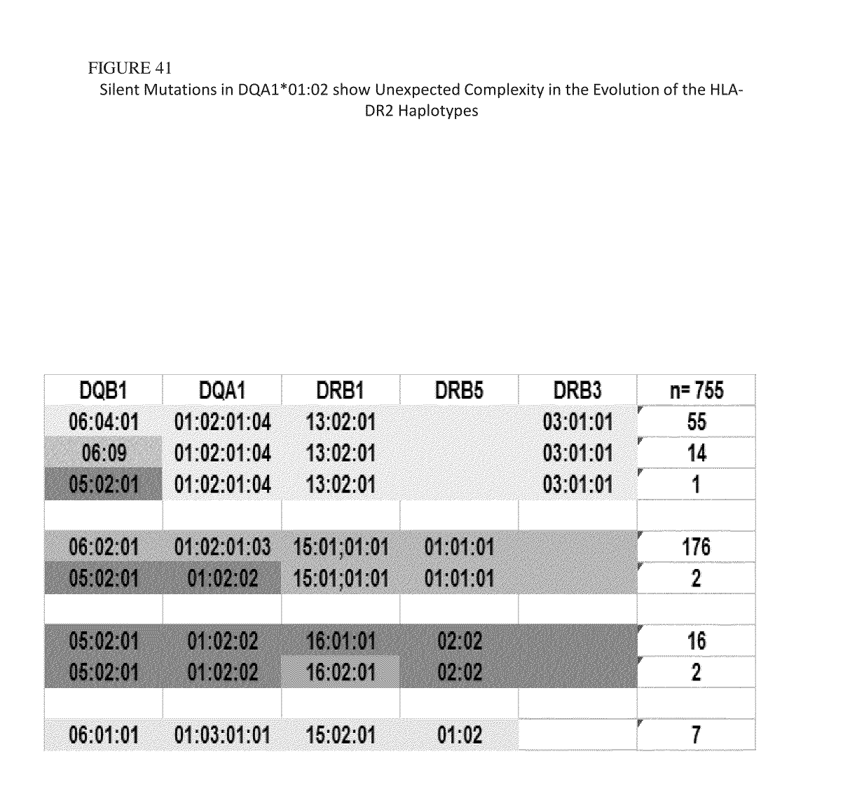

FIG. 42 depicts exemplary homozygous alleles.

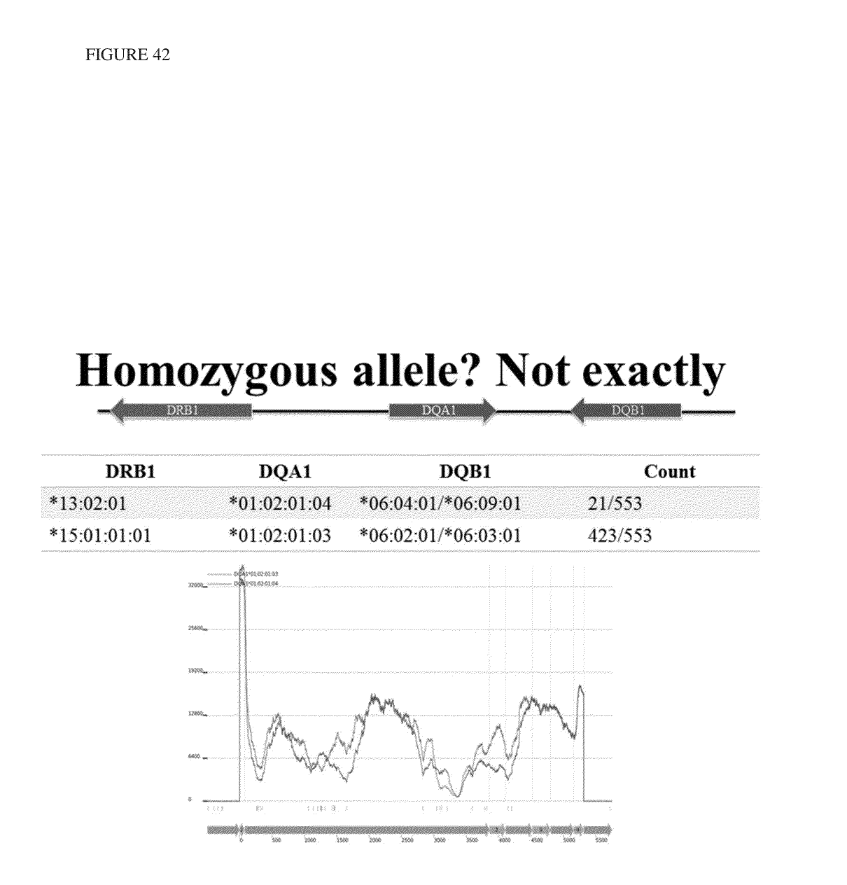

FIG. 43 depicts a coverage graph.

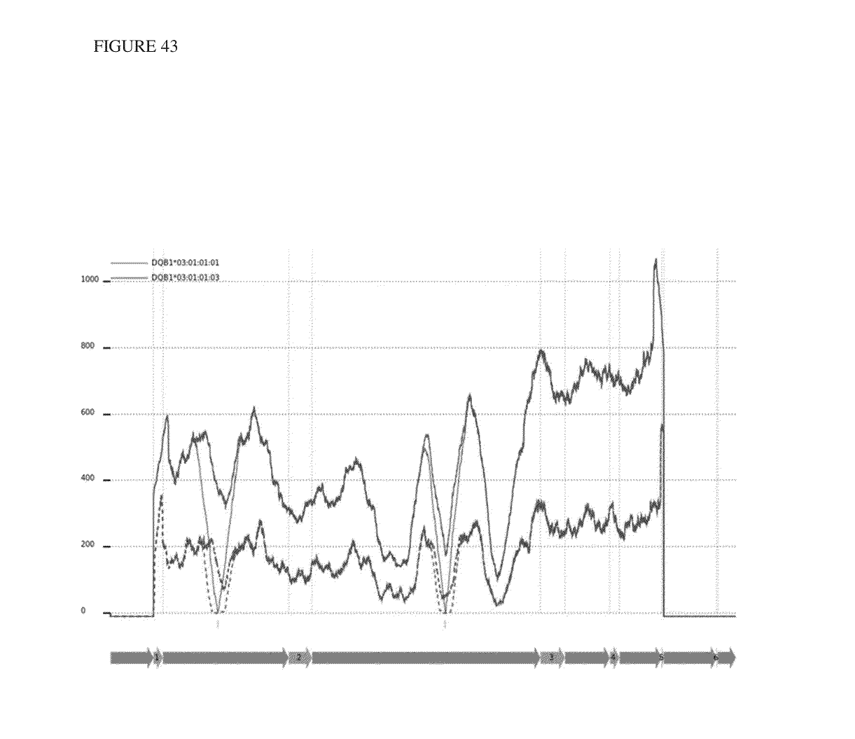

FIG. 44 depicts a coverage graph.

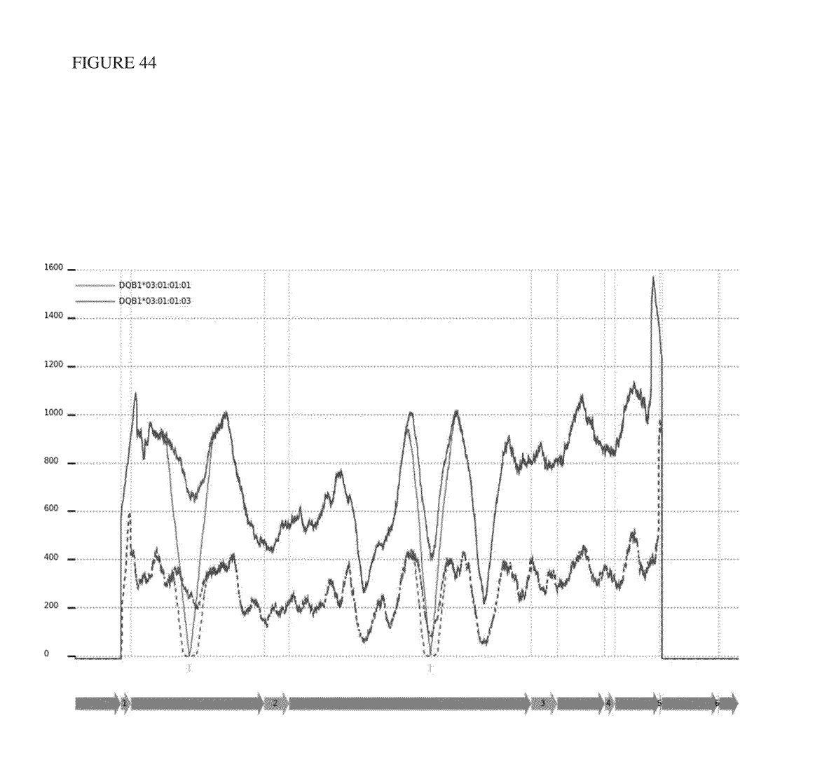

FIG. 45 depicts exemplary silent mutations with multiple mutational events.

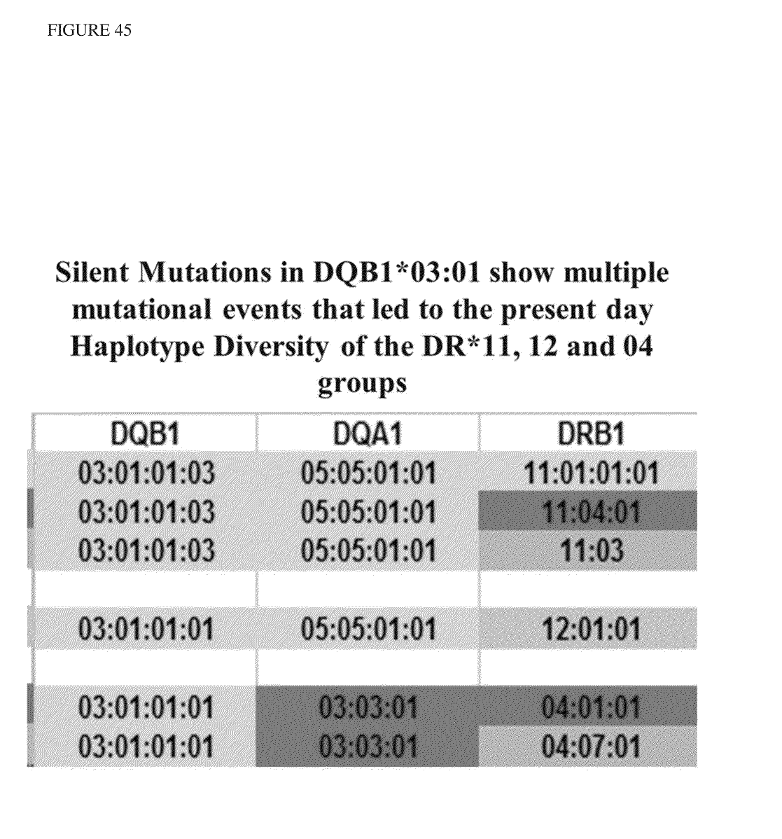

FIG. 46 depicts exemplary multiple mutational events.

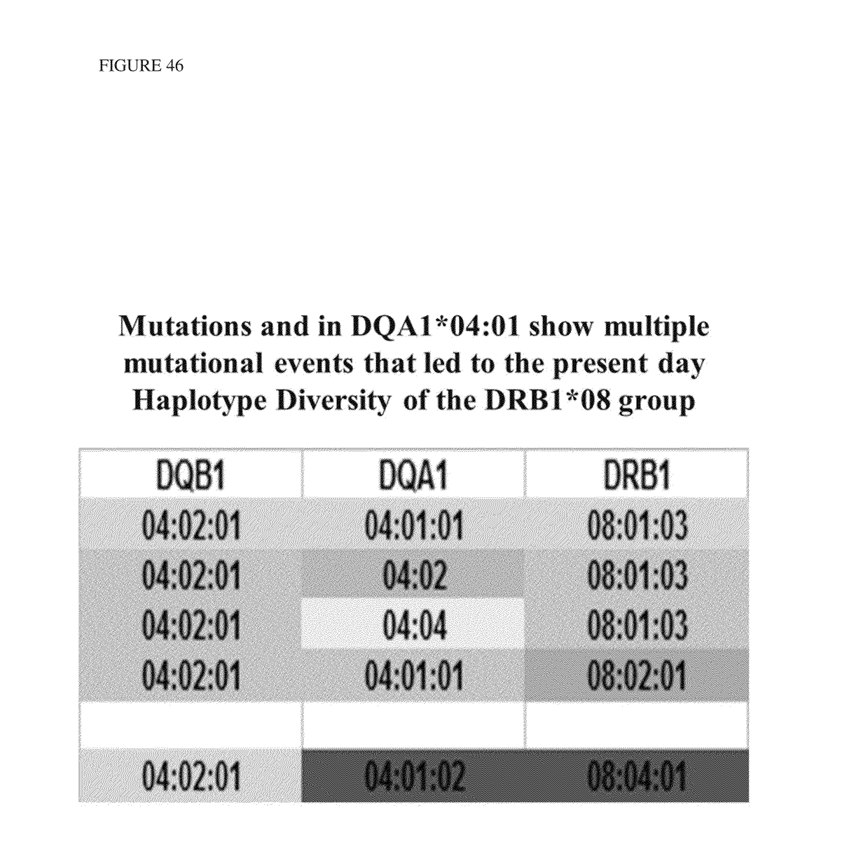

FIG. 47 gives examples of potential erroneous reference sequences.

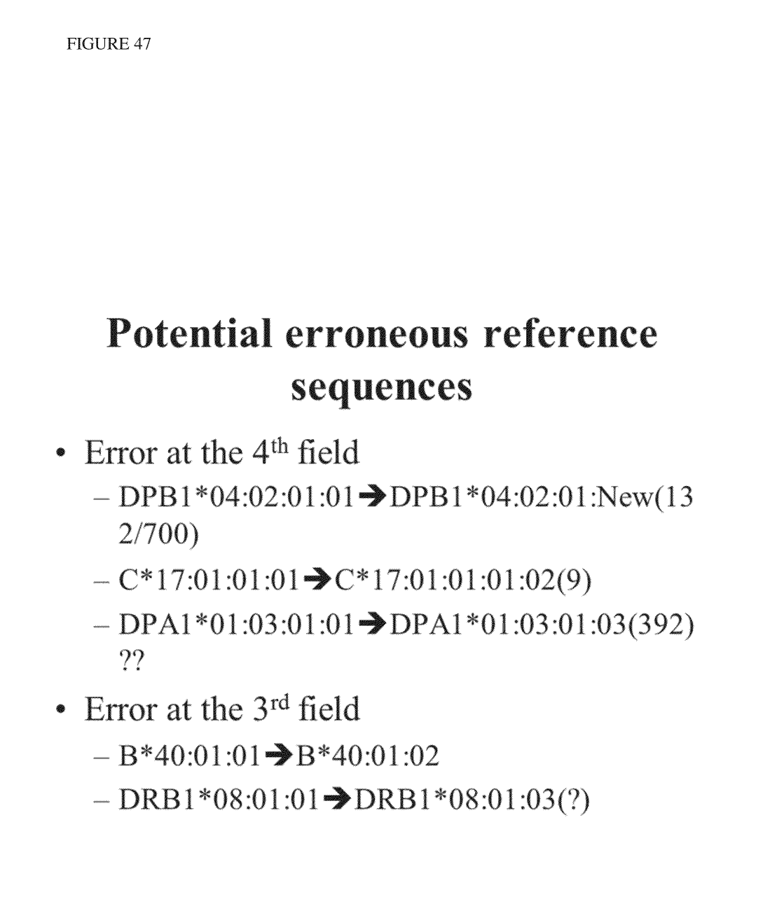

FIG. 48 lists exemplary alleles at the fourth field.

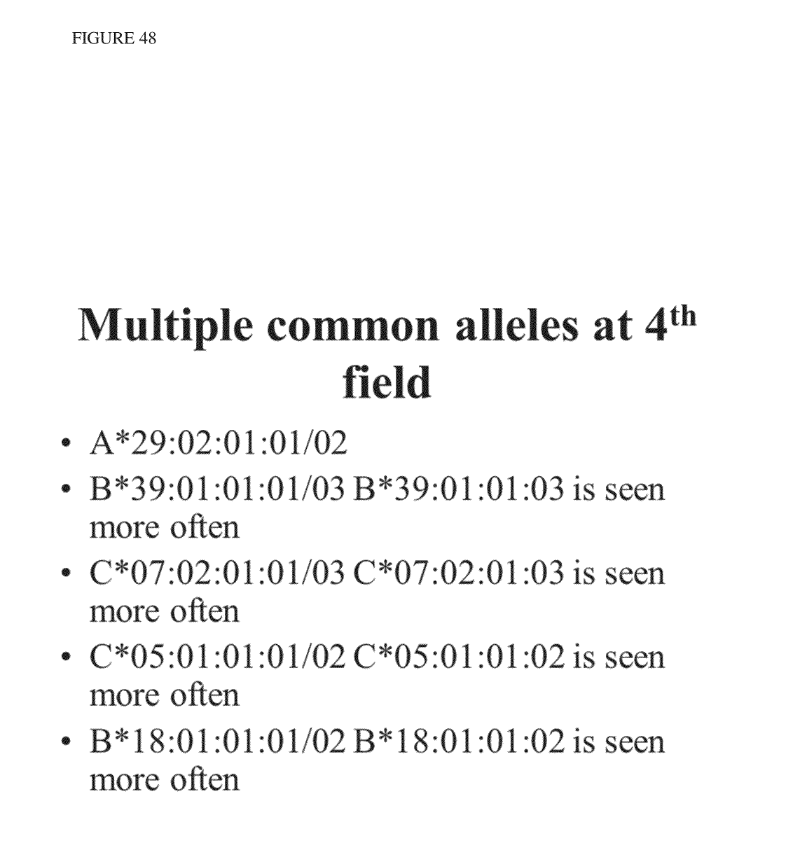

FIG. 49 lists an exemplary rare allele detection sequence.

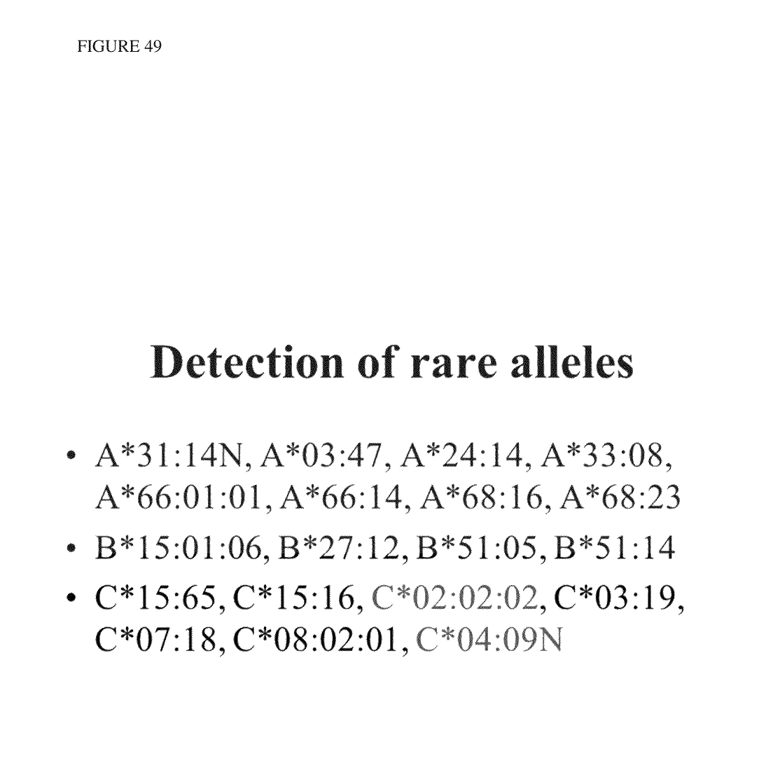

FIG. 50 depicts an exemplary novel allele found.

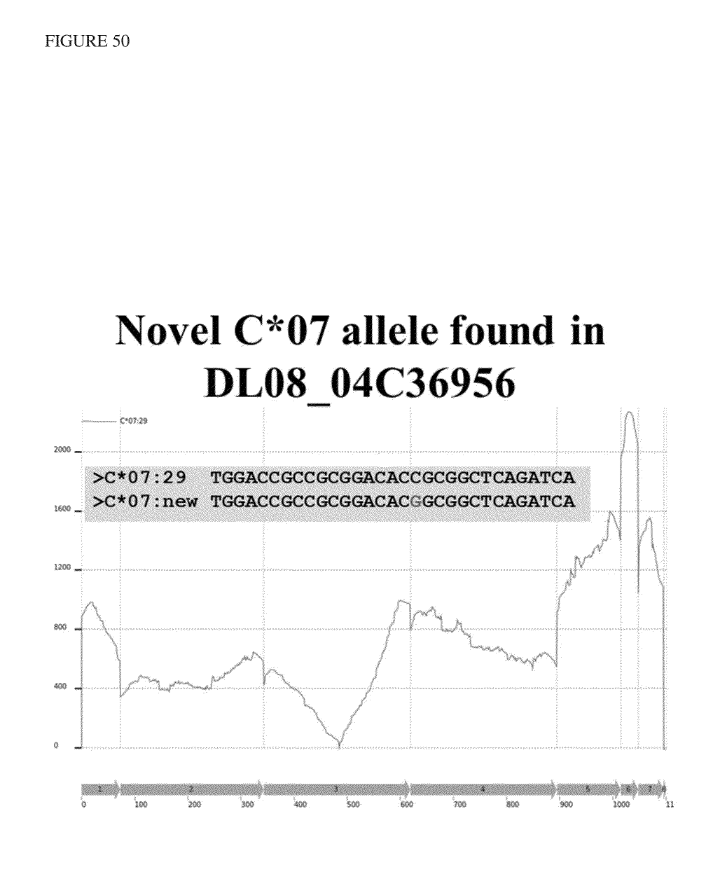

FIG. 51 depicts exemplary allele variants.

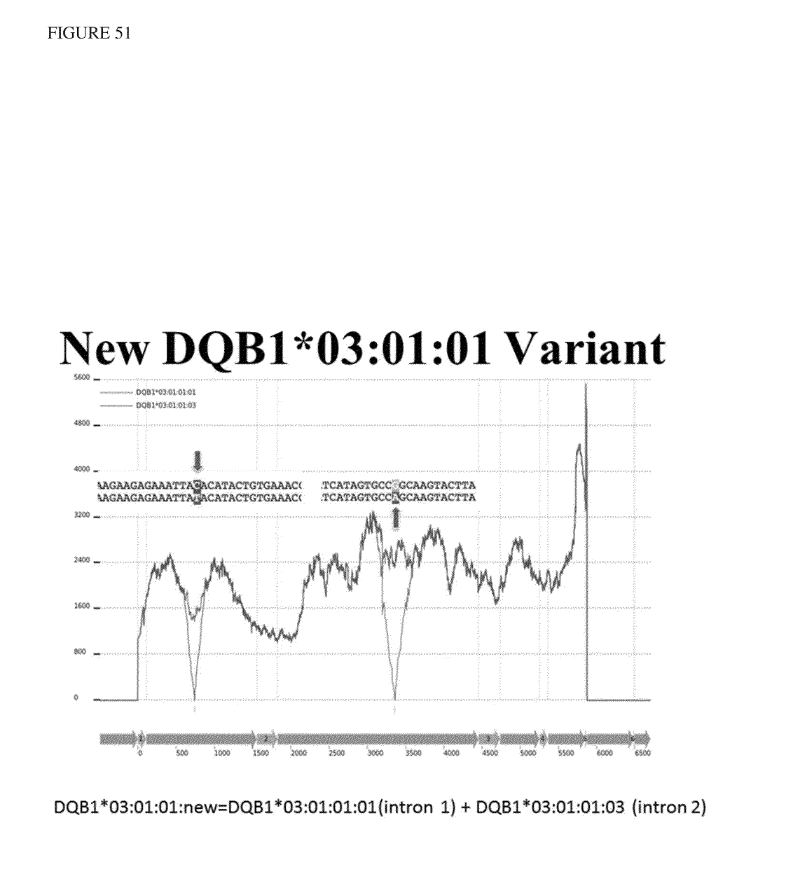

FIG. 52 depicts exemplary allele variants.

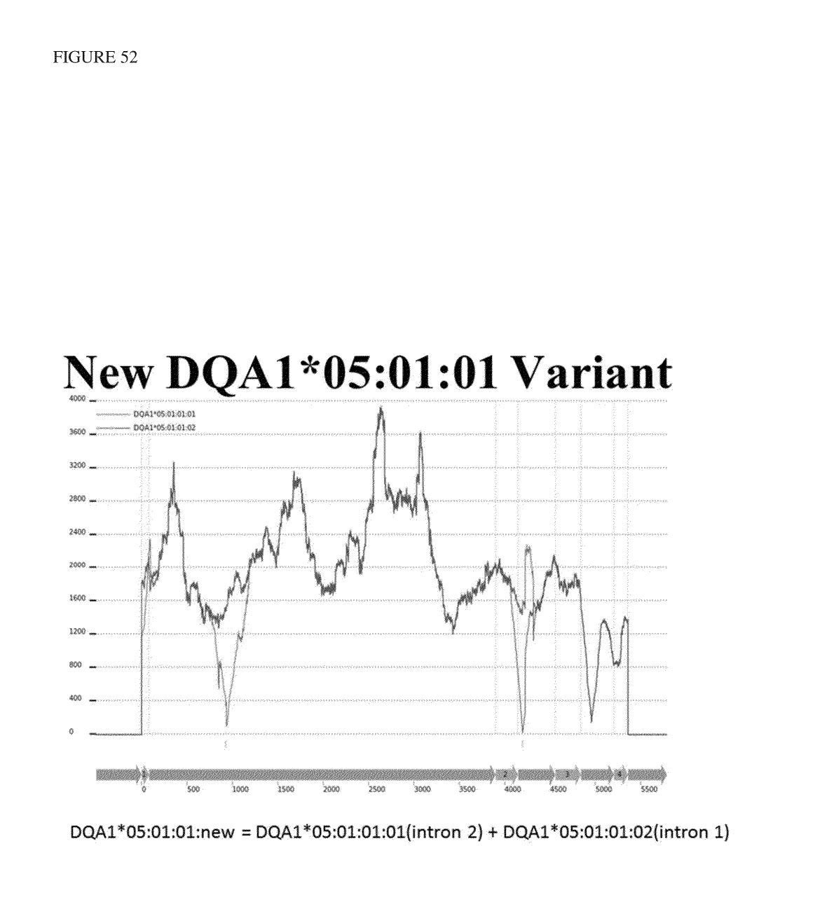

FIG. 53 depicts exemplary LD at fourth fields.

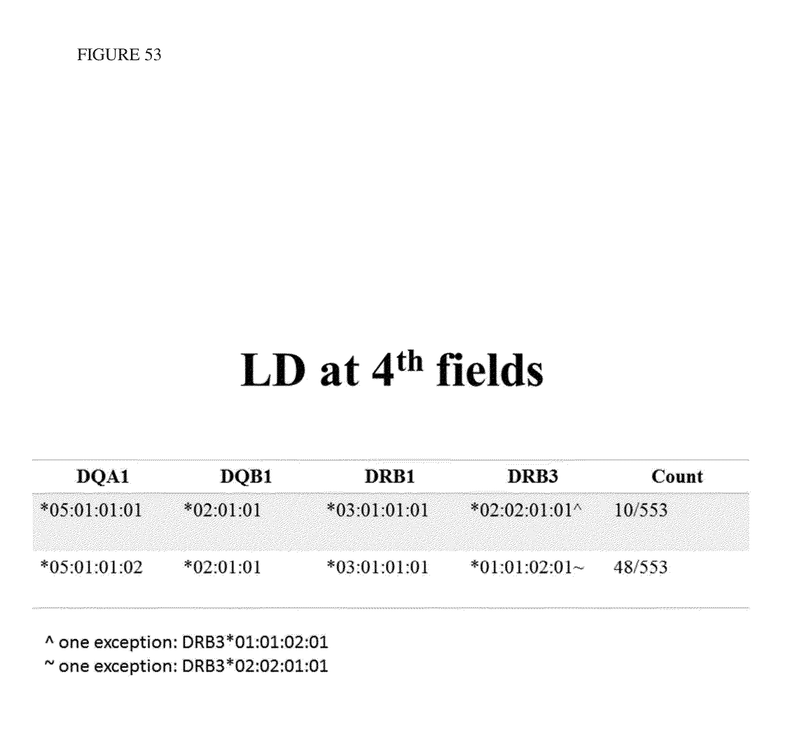

FIG. 54 depicts exemplary LD pattern changes.

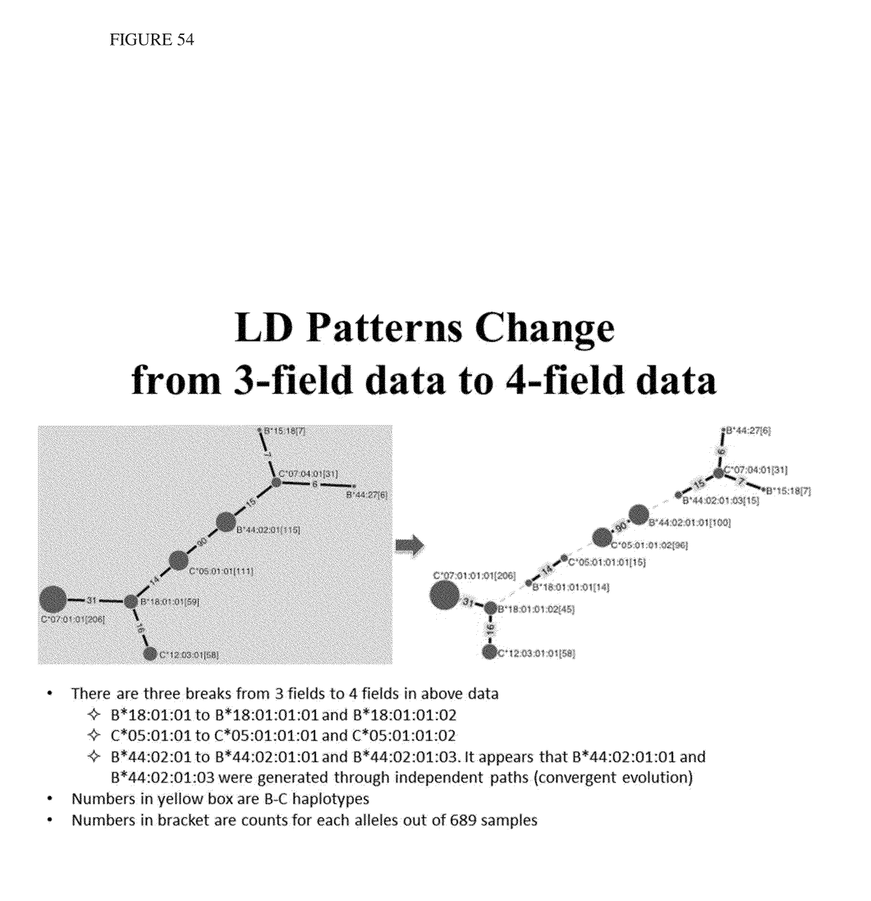

FIG. 55 depicts exemplary amplified signal-vs-noise results.

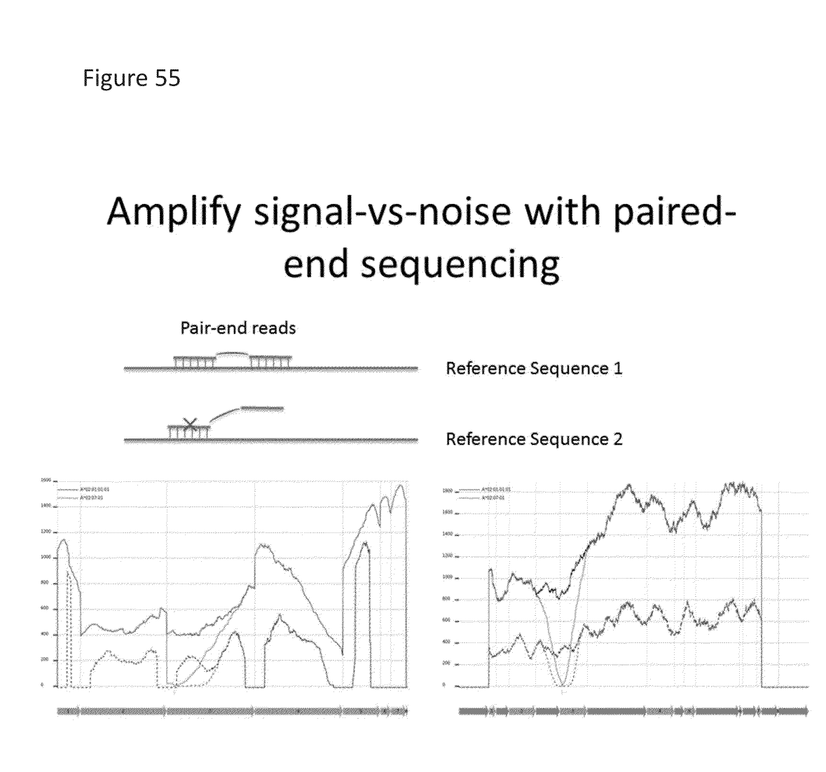

FIG. 56 depicts exemplary use of a paired-end filter.

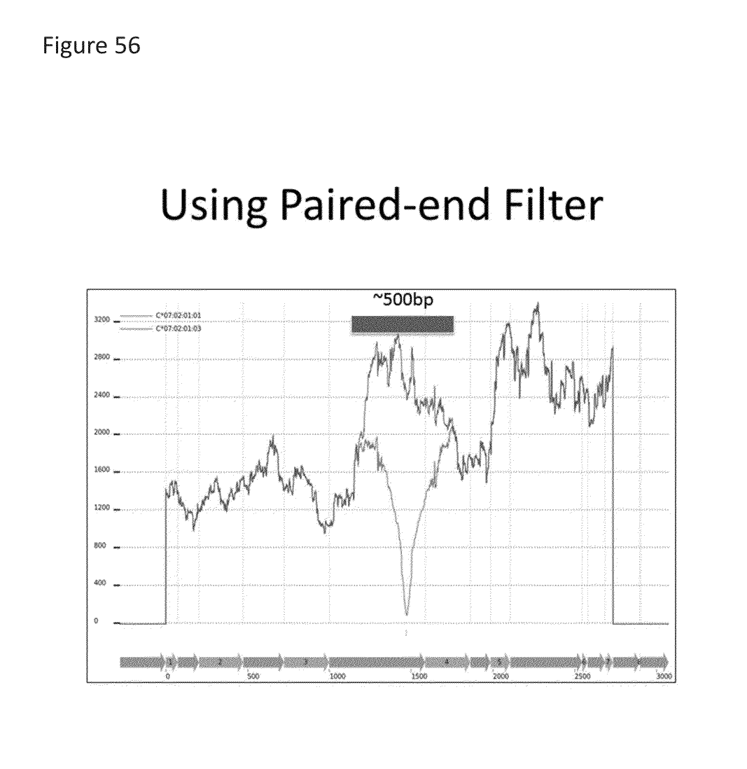

FIG. 57 depicts exemplary central read coverage.

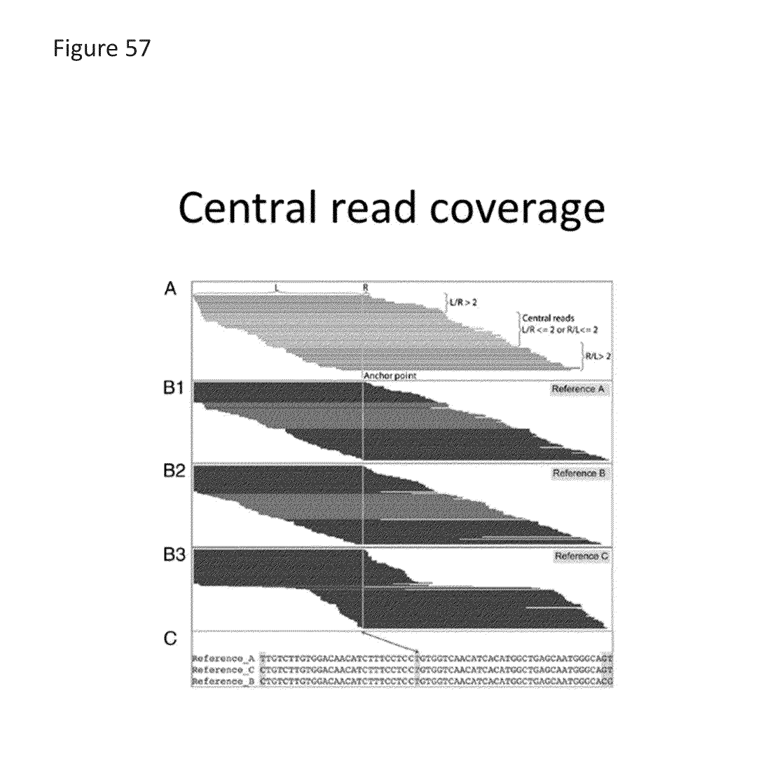

FIG. 58 depicts exemplary use of central reads coverage.

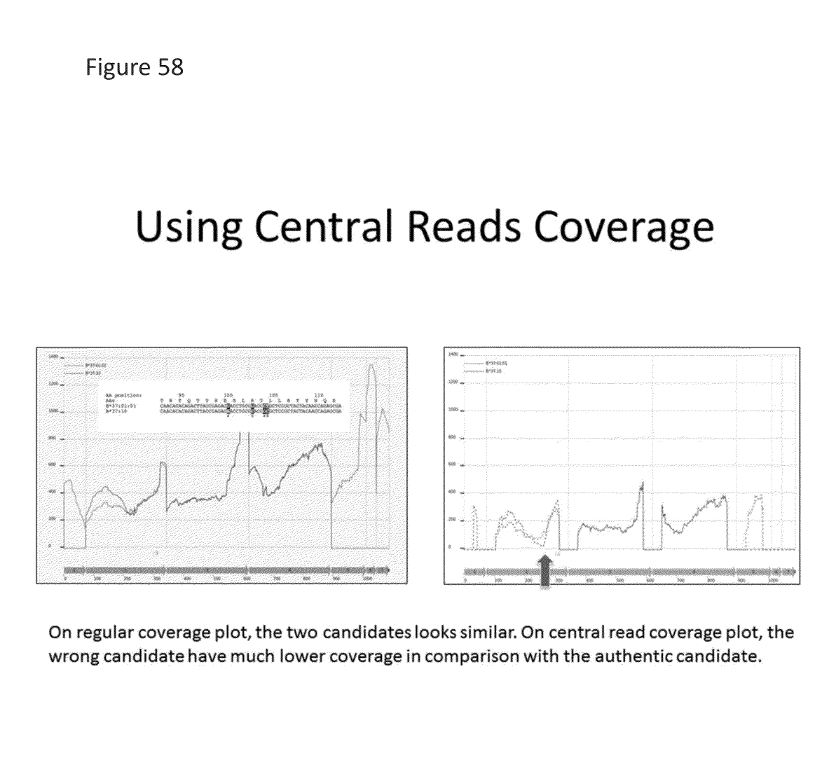

FIG. 59 depicts exemplary complement logics resolved difficult alleles.

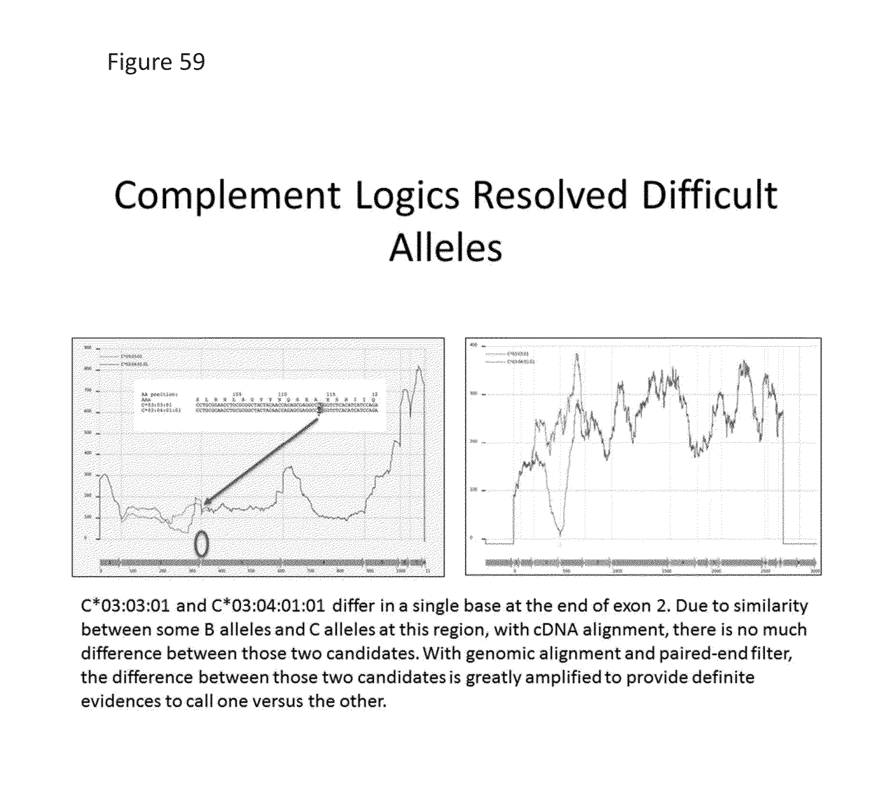

FIG. 60 depicts exemplary use of complement logics.

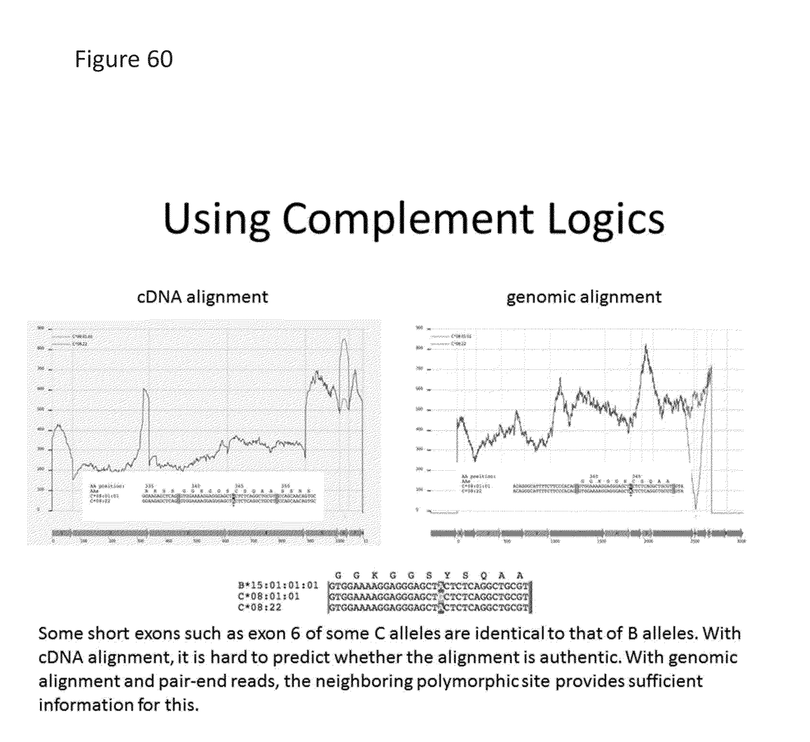

FIG. 61 depicts an exemplary chart of the divide-and-conquer strategy.

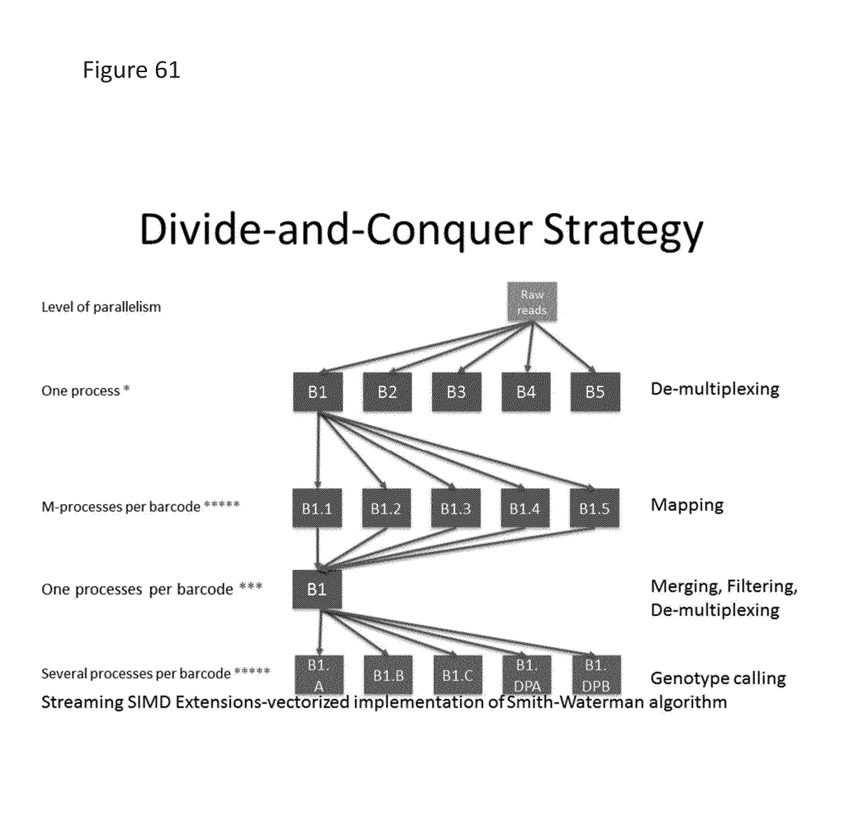

FIG. 62 depicts an exemplary image of the user-friendly interface.

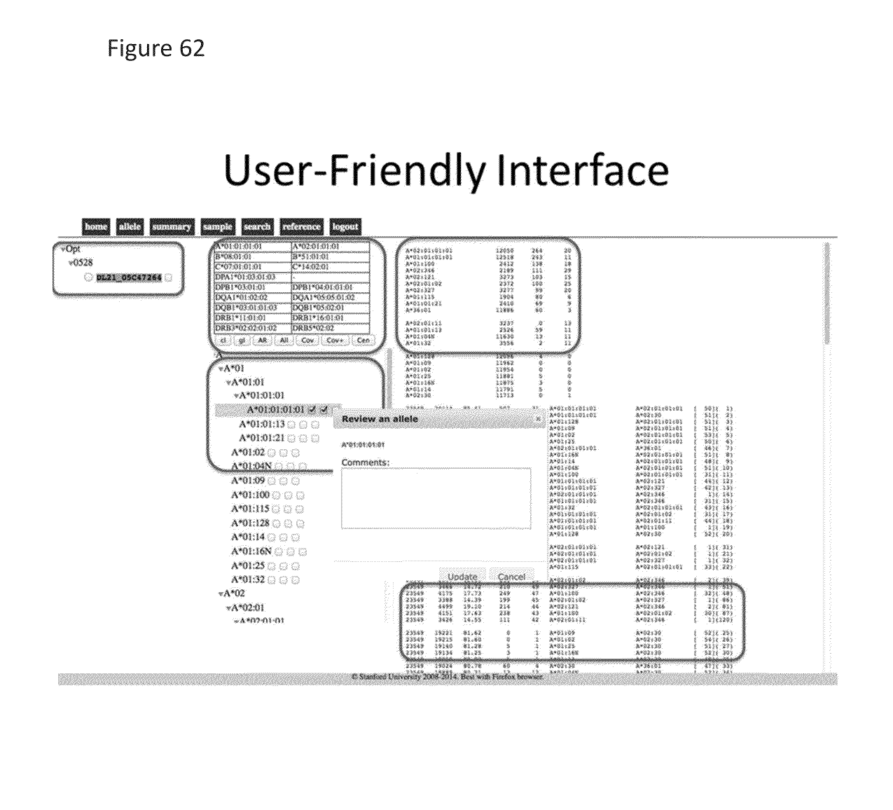

FIG. 63 depicts an exemplary image of the user-friendly interface.

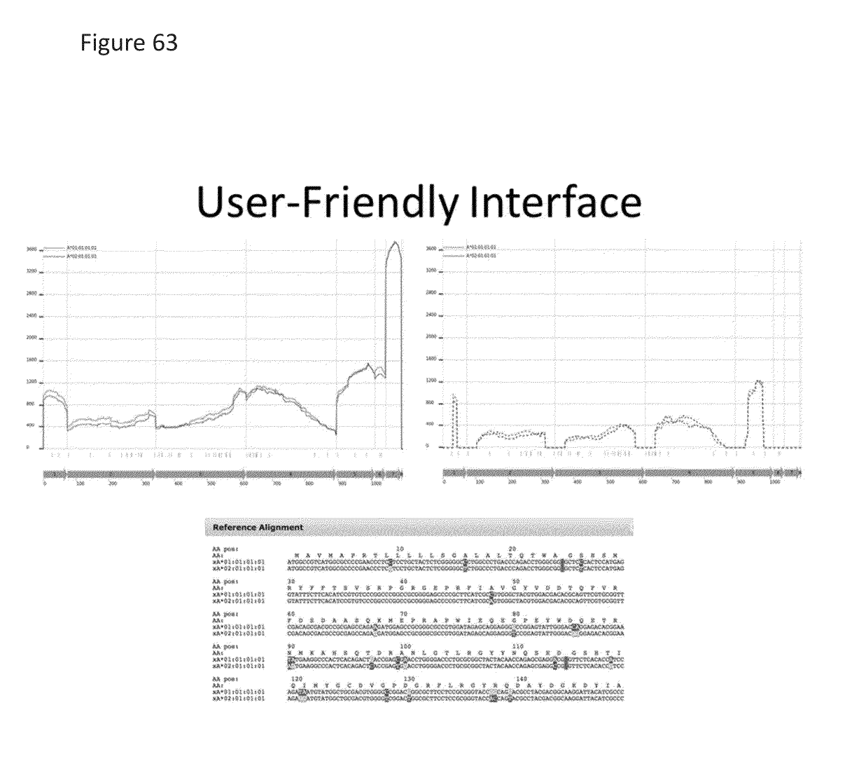

FIG. 64 depicts a table of primers.

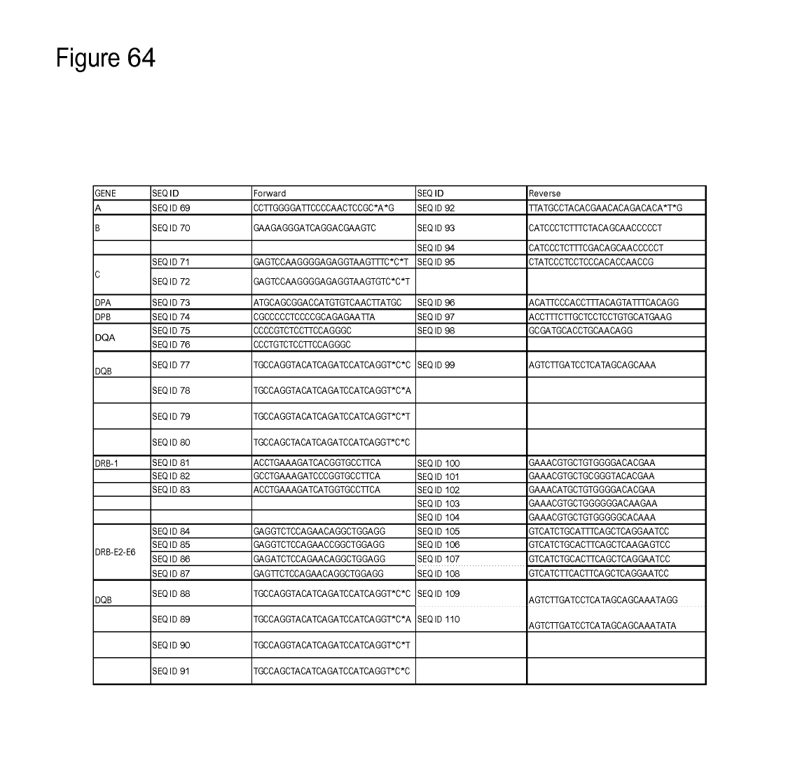

FIG. 65 depicts a table of primers.

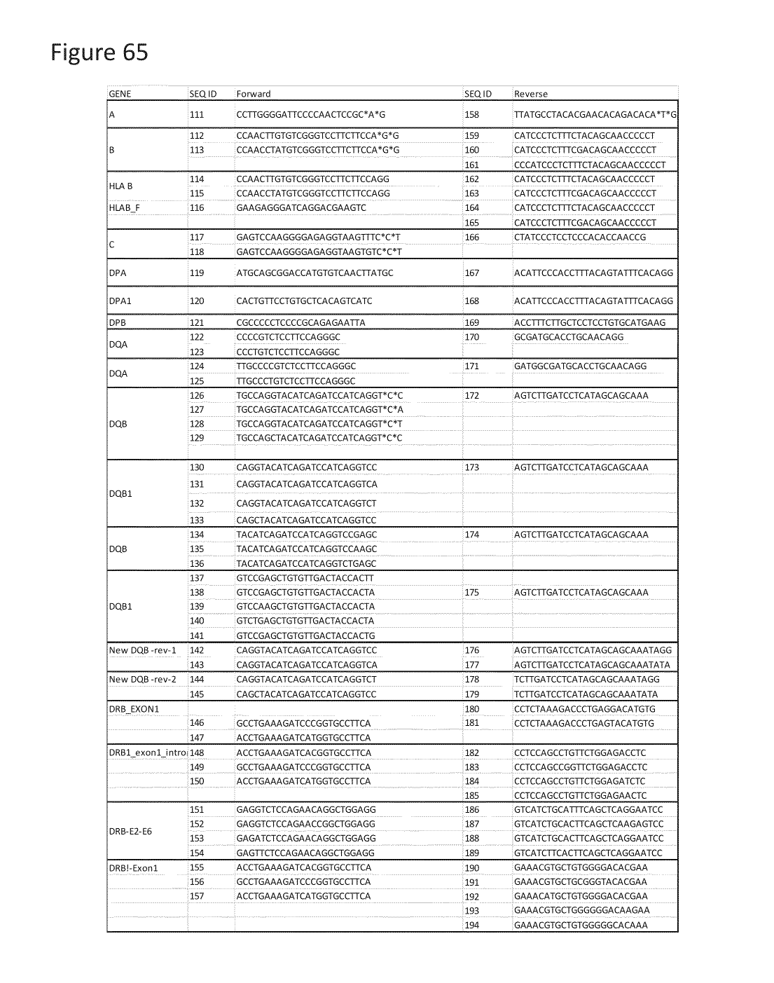

### DETAILED DESCRIPTION

Before the subject invention is described further, it is to be understood that the invention is not limited to the particular embodiments of the invention described below, as variations of the particular embodiments may be made and still fall within the scope of the appended claims. It is also to be understood that the terminology employed is for the purpose of describing particular embodiments, and is not intended to be limiting. In this specification and the appended claims, the singular forms “a,” “an” and “the” include plural reference unless the context clearly dictates otherwise.

Where a range of values is provided, it is understood that each intervening value, to the tenth of the unit of the lower limit unless the context clearly dictates otherwise, between the upper and lower limit of that range, and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, illustrative methods, devices and materials are now described.

All publications mentioned herein are incorporated herein by reference for the purpose of describing and disclosing the subject components of the invention that are described in the publications, which components might be used in connection with the presently described invention.

The present invention has been described in terms of particular embodiments found or proposed by the present inventor to comprise preferred modes for the practice of the invention. It will be appreciated by those of skill in the art that, in light of the present disclosure, numerous modifications and changes can be made in the particular embodiments exemplified without departing from the intended scope of the invention. For example, due to codon redundancy, changes can be made in the underlying DNA sequence without affecting the protein sequence. Moreover, due to biological functional equivalency considerations, changes can be made in protein structure without affecting the biological action in kind or amount. All such modifications are intended to be included within the scope of the appended claims.

### DEFINITIONS

An “allele” can refer to one of the different nucleic acid sequences of a gene at a particular locus on a chromosome. One or more genetic differences can constitute an allele. Examples of HLA allele sequences are set out in Mason and Parham (1998) Tissue Antigens 51: 417-66, which list HLA-A, HLA-B, and HLA-C alleles and Marsh et al. (1992) Hum. Immunol. 35:1, which list HLA Class II alleles for DRA, DRB, DQA1, DQB1, DPA1, and DPB1. Further the International Histocompatibility Working Group (IHWG) has a reference panel.

A “locus” can refer to a discrete location on a chromosome. Exemplary loci can include the class I MHC genes designated HLA-A, HLA-B and HLA-C; nonclassical class I genes including HLA-E, HLA-F, HLA-G, HLA-H, HLA-J and HLA-X, MIC; and class II genes such as HLA-DP, HLA-DQ and HLA-DR.

The MICA (PERB11.1) gene spans an 11 kb stretch of DNA and is approximately 46 kb centromeric to HLA-B. MICB (PERB11.2) is 89 kb farther centromeric to MICA (MICC, MICD and MICE are pseudogenes). Both genes are highly polymorphic at all three alpha domains and show 15-36% sequence similarity to classical class I genes. MIC genes are classified as MHC class Ic genes in the beta block of MHC.

A method of “identifying an genotype” can be a method that permits the determination or assignment of one or more genetically distinct polymorphisms, and where the polymorphisms are assigned to one of the alleles present in an individual.

The term “haplotype” can be used herein to refer to the set of alleles comprising the genotype on one chromatid of the linked genes of the major histocompatibility locus.

The term “amplifying” can refer to a reaction wherein the template nucleic acid, or portions thereof, are duplicated at least once. “Amplifying” may refer to arithmetic, logarithmic, or exponential amplification. The amplification of a nucleic acid can take place using any nucleic acid amplification system, both isothermal and thermal gradient based, including but not limited to, polymerase chain reaction (PCR), reverse-transcription-polymerase chain reaction (RT-PCR), ligase chain reaction (LCR), self-sustained sequence reaction (3 SR), and transcription mediated amplifications (TMA). Typical nucleic acid amplification mixtures (e.g. PCR reaction mixture) include a nucleic acid template that is to be amplified, a nucleic acid polymerase, nucleic acid primer sequence(s), and nucleotide triphosphates, and a buffer containing all of the ion species required for the amplification reaction.

An “amplification product” can be a single stranded or double stranded DNA or RNA or any other nucleic acid products of isothermal or thermal gradient amplification reactions, including PCR, TMA, 3SR, LCR, etc.

The term “amplicon” is used herein to mean a population of nucleic acids that has been produced by amplification, e.g., by PCR.

The phrase “template nucleic acid” refers to a nucleic acid polymer that is sought to be copied or amplified. The “template nucleic acid(s)” can be isolated or purified from a cell, tissue, animal, or amplified product as well etc. Alternatively, the “template nucleic acid(s)” can be contained in a lysate of a cell, tissue, animal, etc. The template nucleic acid can contain genomic DNA, cDNA, plasmid DNA, etc.

The term “dNTP” can be a generic term referring to deoxyribonucelotide triphosphates and can be used to refer to both “regular dNTPs” and “dNTP analogs. The term “regular dNTPs” can be used to refer to the four most common deoxyribonucleotide triphosphates found in nature, including dATP, dCTP, dGTP and dTTP.

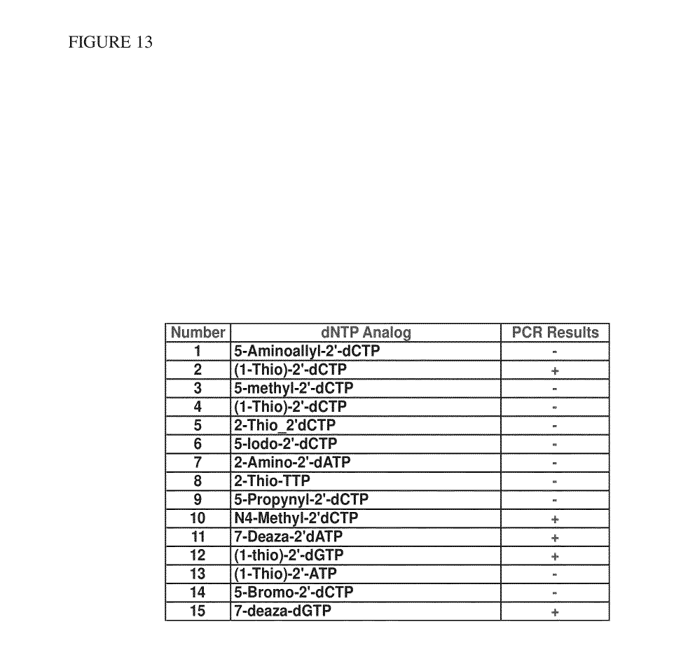

The term “dNTP analog” can refer to a chemical analog of dNTP. The dNTP analog can have a chemical structure similar to that of the corresponding dNTP, but differs from the dNTP in at least one atom or at least one bond type. Some non-limiting examples of dNTP analogs can are listed in the table of FIG. 13.

An “HLA allele-specific” primer can be an oligonucleotide that hybridizes to nucleic acid sequence variations that define or partially define that particular HLA allele.

An “HLA gene-specific” primer can be an oligonucleotide that permits the amplification of a HLA gene sequence or that can hybridize specifically to an HLA gene.

A “forward primer” and a “reverse primer” can constitute a pair of primers that can bind to a template nucleic acid and under proper amplification conditions produce an amplification product. If the forward primer is binding to the sense strand then the reverse primer is binding to antisense strand. Alternatively, if the forward primer is binding to the antisense strand then the reverse primer is binding to sense strand. In essence, the forward or reverse primer can bind to either strand as long as the other reverse or forward primer binds to the opposite strand.

The phrase “hybridizing” can refer to the binding, duplexing, and/or hybridizing of a molecule only to a particular nucleotide sequence or subsequence through specific binding of two nucleic acids through complementary base pairing. Hybridization typically involves the formation of hydrogen bonds between nucleotides in one nucleic acid and complementary sequences in the second nucleic acid.

The phrase “hybridizing specifically” can refer to hybridizing that is carried out under stringent conditions.

The term “stringent conditions” can refer to conditions under which a capture oligonucleotide, oligonucleotide or amplification product will hybridize to its target subsequence, but to no other sequences. Stringent conditions are sequence-dependent and will be different in different circumstances. Longer sequences hybridize specifically at higher temperatures. Generally, stringent conditions are selected to be about 5° C. lower than the thermal melting point (Tm) for the specific sequence at a defined ionic strength and pH. The Tm is the temperature (under defined ionic strength, pH, and nucleic acid concentration) at which 50% of the probes complementary to the target sequence hybridize to the target sequence at equilibrium. (As the target sequences are generally present in excess, at Tm, 50% of the capture oligonucleotides are occupied at equilibrium). Typically, stringent conditions will be those in which the salt concentration is at most about 0.01 to 1.0 M Na⁺ ion concentration (or other salts) at pH 7.0 to 8.3 and the temperature is at least about 30° C. for short probes (e.g., 10 to 50 nucleotides). Stringent conditions may also be achieved with the addition of destabilizing agents such as formamide. An extensive guide to the hybridization and washing of nucleic acids is found in Tijssen (1993) Laboratory Techniques in biochemistry and molecular biology—hybridization with nucleic acid probes parts I and II, Elsevier, N.Y., and, Choo (ed) (1994) Methods In Molecular Biology Volume 33-In Situ Hybridization Protocols Humana Press Inc., New Jersey; Sambrook et al., Molecular Cloning, A Laboratory Manual (2^{nd} ed. 1989); Current Protocols in Molecular Biology (Ausubel et al., eds., (1994)).

The term “complementary base pair” refers to a pair of bases (nucleotides) each in a separate nucleic acid in which each base of the pair is hydrogen bonded to the other. A “classical” (Watson-Crick) base pair always contains one purine and one pyrimidine; adenine pairs specifically with thymine (A-T), guanine with cytosine (G-C), uracil with adenine (U-A). The two bases in a classical base pair are said to be complementary to each other. Base pairs can also hydrogen bond to nucleotide analogs.

The term “portions” should similarly be viewed broadly, and would include the case where a “portion” of a DNA strand is in fact the entire strand.

The term “specificity” refers to the proportion of negative test results that are true negative test result. Negative test results include false positives and true negative test results.

The term “sensitivity” is meant to refer to the ability of an analytical method to detect small amounts of analyte. Thus, as used here, a more sensitive method for the detection of amplified DNA, for example, would be better able to detect small amounts of such DNA than would a less sensitive method. “Sensitivity” refers to the proportion of expected results that have a positive test result.

The term “reproducibility” as used herein refers to the general ability of an analytical procedure to give the same result when carried out repeatedly on aliquots of the same sample.

### Methods and Compositions

Compositions and methods are provided for accurately determining the gene sequence of highly polymorphic genes (e.g. the HLA genotype of an individual). The methods of the invention can comprise the steps of: amplifying HLA regions (e.g. multiple introns and exons of an HLA gene in a single, long-range reaction); sequencing the amplified genomic regions (e.g. by deep sequencing or NGS sequencing methods); and performing analysis (e.g. deconvolution analysis to resolve the genotype of each allele). The methods of the invention thus determine the genomic sequence of both alleles at a particular HLA gene, including both intron and exons. The resultant consensus sequence from each of the analyzed loci provides linkage information between different exons, and is used to produce the unique sequence from each allele of the gene. The sequence information in intron regions, along with the exon sequences, provides an accurate HLA genotype, which can be critical to solve phasing problems that current HLA haplotyping approaches have thus far failed to address.

In some embodiments, the methods described herein can be advantageous over previously known methods in the art. For example, the methods of the disclosure can use the Illumina NGS platform with consistent performance. The methods of the disclosure can use the Illumina NGS platform with a reduced error rate. The methods of the disclosure can be adaptable for both high and low throughput. Some non-limiting examples of throughput, as measured from sample to results, can be follows: about 16 to about 24 samples for all HLA loci in about 4 to about 5 days; about 192 to 768 samples for all loci in about 1 week; about 3072 samples for all loci in about two weeks. One skilled in the art will recognize that these examples of scalable throughput are only intended as an example and demonstrate the scalability of the protocol.

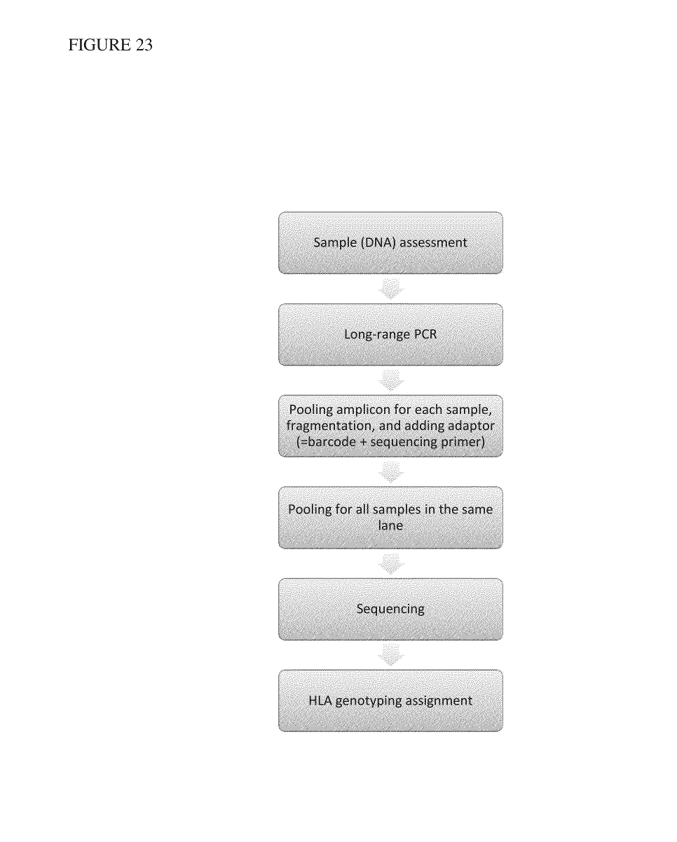

The process work flow can be automated. In some embodiments, the methods are advantageous over previously known methods because the methods can be semi or fully automated. The methods described herein can be advantageous because of cost-effectiveness (e.g. lower cost via multiplexed NGS was previously not possible using Sanger-based sequencing methods). FIG. 23 depicts an exemplary process workflow. Automation can occur throughout the workflow. For example, the long-range PCR step can be automated; the pooling and fragmentation step can be automated and the like.

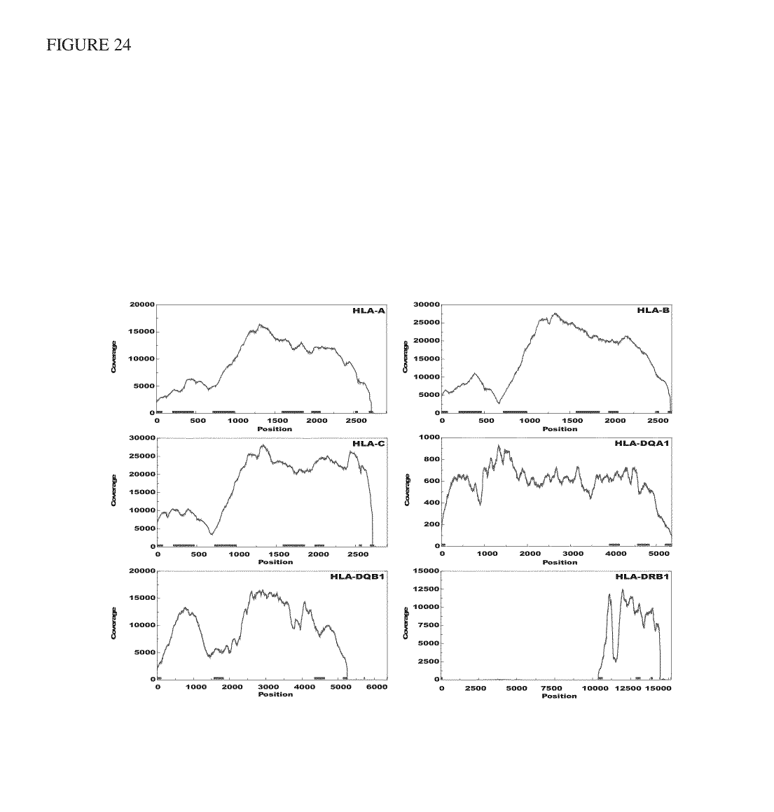

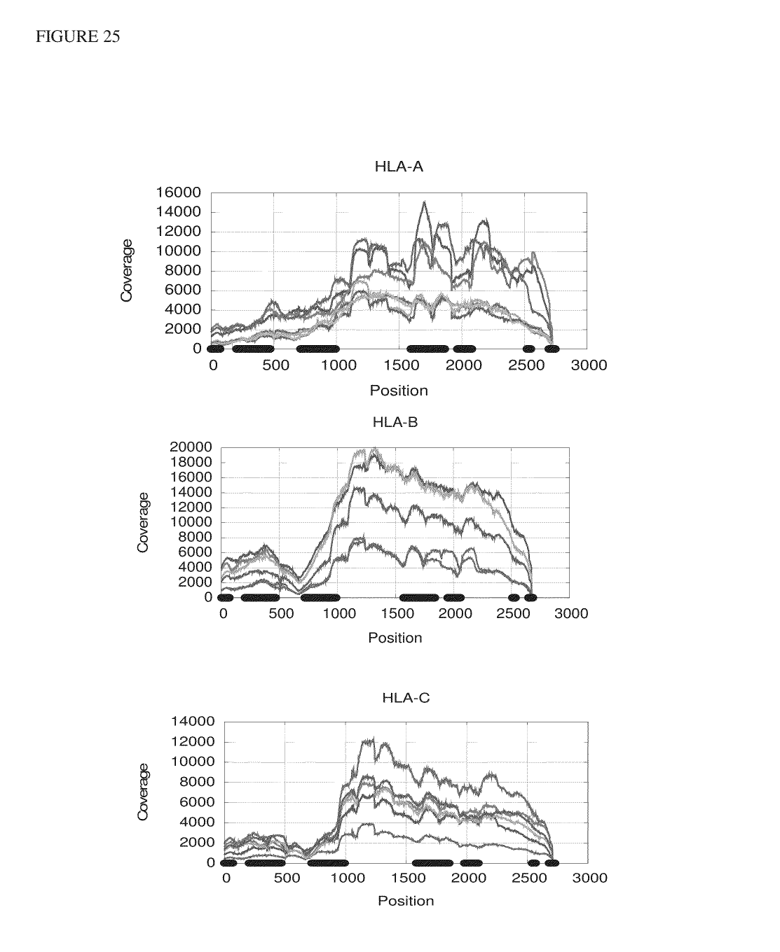

The methods described herein can be preferred over current HLA-typing methods. For example, standard HLA typing methods have resulted in incomplete coverage of important HLA loci and gene segments (e.g. resulting in invalid assumptions that lead to undetected functional differences or mismatches). In one non-limiting example, each mismatch can reduce the success rate of a bone marrow transplant by 22%. In another example, the lower-resolution results used to type HLA can result in a longer matching process because multiple donors may need to be evaluated. FIGS. 24 and 25 show exemplary experimental data demonstrating the reproducibility of coverage using an embodiment of methods described herein.

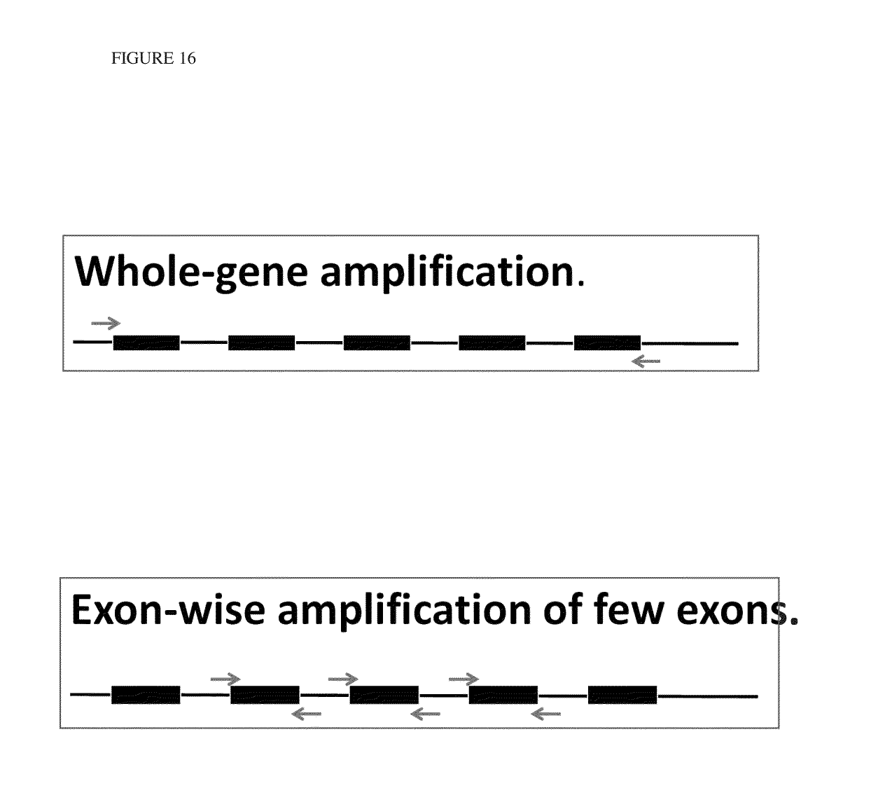

The methods described herein can employ a whole gene amplification strategy. In some instances, a whole gene amplification strategy can be preferable over an exon-wise amplification of a few genes. An exemplary illustration of the difference between exon-wise amplification and whole-gene amplification can be seen in FIG. 16.

In particular, the methods of the present invention can be useful for determining HLA genotypes of samples. Samples can be from subjects. Samples can be from non-human organisms such as: bacteria, insects, non-vertebrates, vertebrates, amphibians, birds, reptiles, mammals and the like. Subjects can be human or non-human. Some examples of non-human subjects can include pets and farm animals. Such genotyping is important in the clinical arena (e.g. for the diagnosis of disease, transplantation of organs, and bone marrow and cord blood applications and for disease-association studies).

A DNA sample can be obtained from any suitable cell source, (e.g. blood, saliva, skin, etc.). Suitable samples may be fresh or frozen, and extracted DNA may be dried or precipitated and stored for long periods of time.

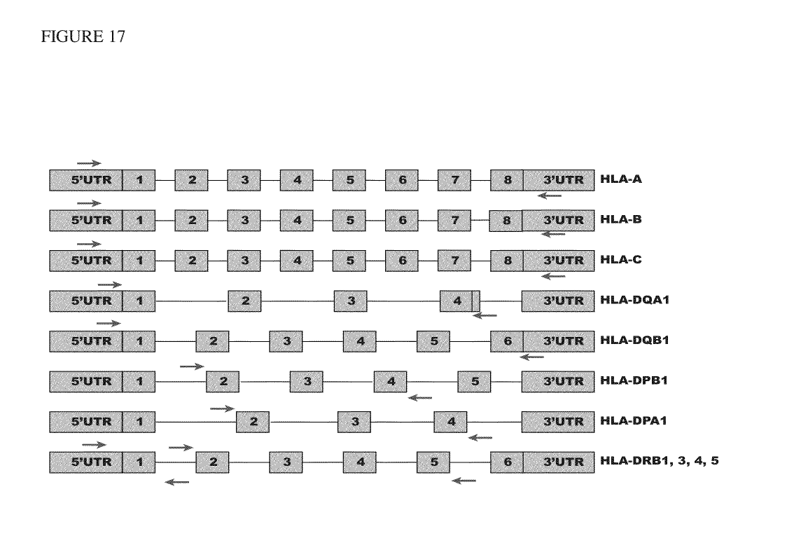

Suitable sets of primers can be used for obtaining high throughput sequence information for genotyping. Sequencing can be performed on sets of nucleic acids across many individuals or on multiple loci in a sample obtained from one individual. Primers can be designed based on an assay design strategy. One non-limiting example of an assay design strategy for use typing HLA genes is depicted in FIG. 17. In the assay development strategy depicted in FIG. 17, long range PCR can be used. Long range PCR can be used, e.g. to capture target regions, including regions that are upstream and/or downstream to the region of interest. In some embodiments, it is both upstream and downstream regions can be included.

The assay design strategy can involve several variables, including: primer design, use of dNTP analogs, use of polymerase, PCR reaction conditions (i.e. including the use of chemicals in the reaction mix, and Tm), the use of downstream software and/or the like. An assay design strategy can also incorporate specificity (e.g. through primer design). The assay design strategy can be used to preserve the specificity and allelic balance. The assay design strategy can be used to effect coverage variance. The assay design strategy can be used to increase reproducibility. The assay design strategy can be used to improve accuracy over conventional HLA typing methods. The assay design strategy can be used to substantially enhance allele resolution. The assay design strategy can be used to dramatically improve combination resolution. The assay design strategy can be used to cover certain gene regions (e.g. all major HLA gene regions). Major HLA gene regions can include: HLA-A, -B, -C, HLA-DPA1 HLA-DPB1, -DQA1 HLA-DQB1, and HLA-DRB1/3/4/5. Coverage of major HLA gene regions can include, for example, HLA-A, -B, -C, including all exons, introns and 5′ and 3′ UTR; HLA-DPA1 and -DQA1, including all exons and introns; HLA-DQB1, including all exons and introns except intron 5 and exon 6; HLA-DRB1, 3/4/5, including all exons and introns except part of introns 1 and 5 and exon 6; and HLA-DPB1, including all exons and introns except exons 1 and 5 and introns 1 and 4.

### Primer Design

The sequences of many HLA alleles are publicly available through GenBank and other gene databases such as IMGT/HLA database and have been published. In the design of the HLA primer pairs, primers can be selected based on the known HLA sequences available in the literature. Those of skill in the art will recognize that a multitude of oligonucleotide compositions that can be used as HLA target-specific primers. Primers can be designed such that the entire gene is amplified. Primers may amplify the entire gene for class I genes (e.g. HLA-A, HLA-B, and HLA-C). Primers may amplify the entire gene for class II genes (e.g. HLA-DQA and HLA-DQB).

Homogeneous PCR conditions were developed to amplify all targeted HLA genes in a uniform PCR conditions to simplify sample process protocol.

Primers can be made to contain at least one dNTP analog. Two or more dNTP analogs can be used in primers. Primers can contain two or more dNTP analogs that are the same. Primers can contain two or more dNTP analogs that are different. A primer pair can contain at least one or more dNTP analogs. Forward and reverse primers can contain the same or different dNTP analogs. These primers may amplify the entire gene for the class I genes (HLA-A, HLA-B, and HLA-C) and two class II genes (HLA-DQA and HLA-DQB).

The primers can be specific. A combination of specific forward and reverse primers together can be used. The primers can specifically hybridize to a specific region of template nucleic acid. Specific primers can be used to amplify a specific region of target nucleic acid, as discussed herein. In one non-limiting example, a plurality of gene-specific primers can be used. Some non-limiting examples of primers that can be used to amplify HLA are shown in Table 1. Primers comprising the sequences disclosed in Table 1 can be used to amplify HLA genes. For example, one skilled in the art will recognize that in some instances, nucleotides can be added to primers (e.g. barcodes, adapters, dNTP analogs, restriction enzyme sites, hairpins, etc) without substantially affecting the utility.

Primers can be designed such that an entire genomic area can be amplified in a single reaction. Gene-specific primers are can be designed to hybridize to the regions flanking a gene target. In some embodiments, nested PCR amplification can be performed (e.g. where each target loci is amplified using two or more sets of primers. The primers can be designed to hybridize to regions outside of regions of high variability. Multiple primers can be included in a reaction (e.g. to ensure amplification of all known alleles for each gene).

Primers can be designed to prevent allele drop out. Primers can contain one or more dNTP analogs. Primers can be designed to hybridize to specific regions to prevent allelic drop out. The molarity ratio of primers can be varied to prevent allele drop out.

### Amplification

The methods of the invention can comprise an amplification step. An amplification step may comprise an amplification of template nucleic acid. For example, genomic DNA obtained from a tissue sample may be used as template nucleic acid in a PCR reaction. The amplification step can comprise the use of primers to amplify a genomic region.

In some embodiments, some of HLA genes like DRB gene are amplified in two or more independent PCR reactions. The region of template to be amplified can be long. In instances where the target region is long, a long-range PCR reaction can be used (e.g. to generate long amplicons). A long-range PCR reaction can be used to amplify long and/or polymorphic regions. For HLA-typing, long-range PCR can be preferable because the length of a HLA gene is typically longer than the upper threshold for accepted template nucleic acid in many PCR protocols. In one non-limiting example, a Klenow-based PCR process can generate products on the range of about 400 base pairs. However, the length for class I HLA genes is about 3.5 kilobases; the length for class II HLA genes is about 5-7 kilobases; and the difference between HLA alleles may be about 0.5 kb. Fidelity and/or yield of PCR products can be increased by using long-range PCR methods. In some embodiments, genes such as HLA-DRB1, HLA-DRB3, HLA-DRB4, and HLA-DRB5 may need at least two PCR reactions (e.g. exon 1 is too long).

Long-range PCR amplification can be accomplished with a polymerase enzyme. Some non-limiting examples of commercially available enzymes that can be used in a long-range PCR reaction include: Expand Long Range Template PCR (Roche); Fidelity Taq Polymerase (USB); Crimson Taq (NEB); Q5 and Q5 Hot Start High Fidelity DNA Polymerase (NEB); TAKARA LA Taq; AccuPrime pfx DNA polymerase (Invitrogen); Phire Hot Start II (ThermoFisher); Crimson Long AMP Taq DNA Polymnerase (NEB); Bioline Ranger DNA Polymerase; Bioline Velocity DNA Polymerase; One Taq 2×MM DNA Polymerase (NEB); KAPA Long Range Hot Start Readymix with dye; Extensor HF PCR MM (Thermo Scientific); Master AMP Extra Long PCR (Epicentre); Dynazyme EXT DNA Polymerase (Thermo Scientific); Qiagen Long Range PCR Kit (QIAGEN); and Phire Hot Start II DNA Polymerase (Thermo Scientific); LongAmp Taq DNA Polymerase (i.e. blend of Taq and Deep VentR™ DNA Polymerases).

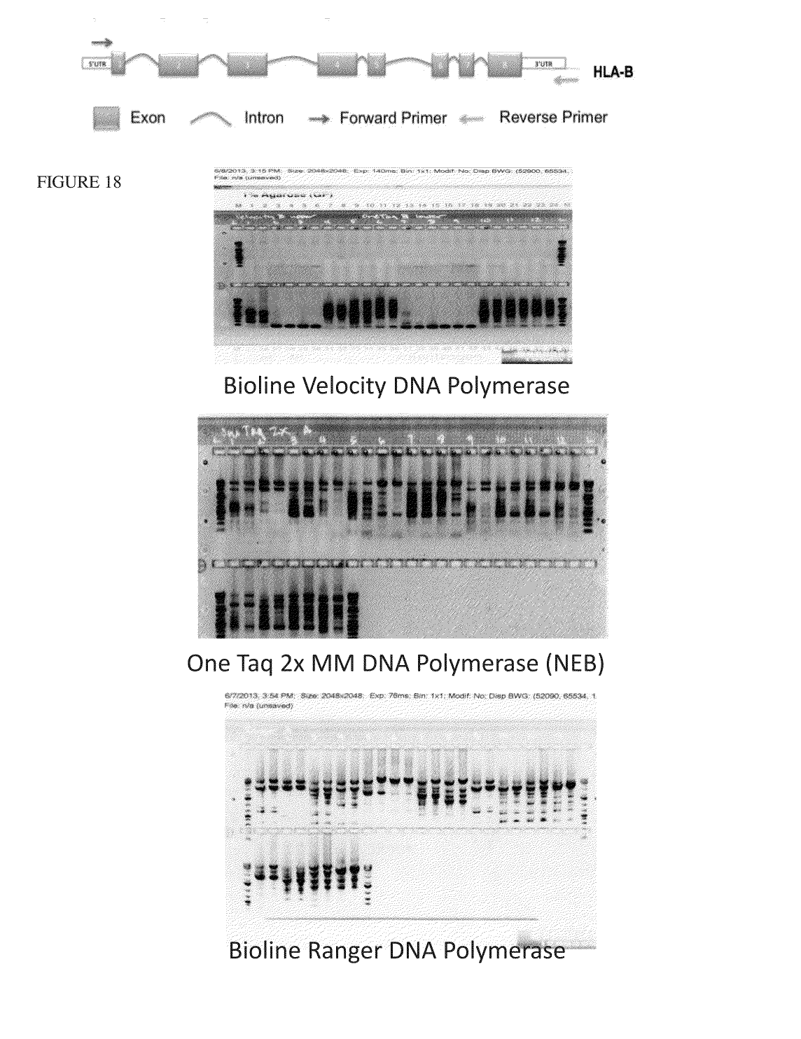

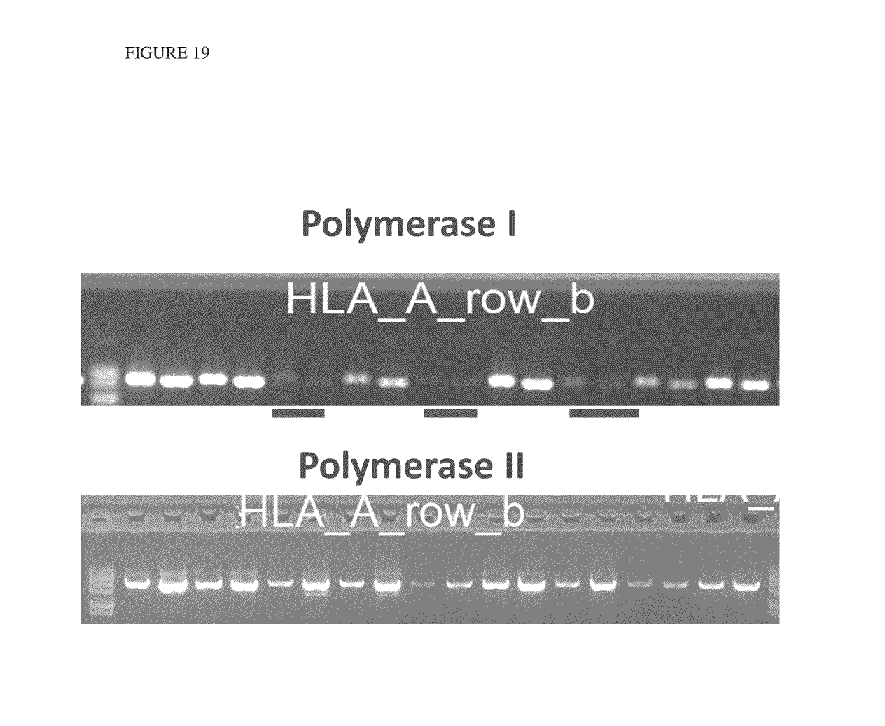

The polymerase used can have an effect on the reaction. FIG. 18 depicts exemplary results comparing the ability of different polymerases to amplify HLA-B. The polymerases compared in FIG. 18 include: Bioline Velocity DNA Polymerase, One Taq 2×MM DNA Polymerase, and Bioline Ranger DNA Polymerase. FIG. 19 depicts exemplary results comparing the ability of different enzymes to amplify HLA-A.

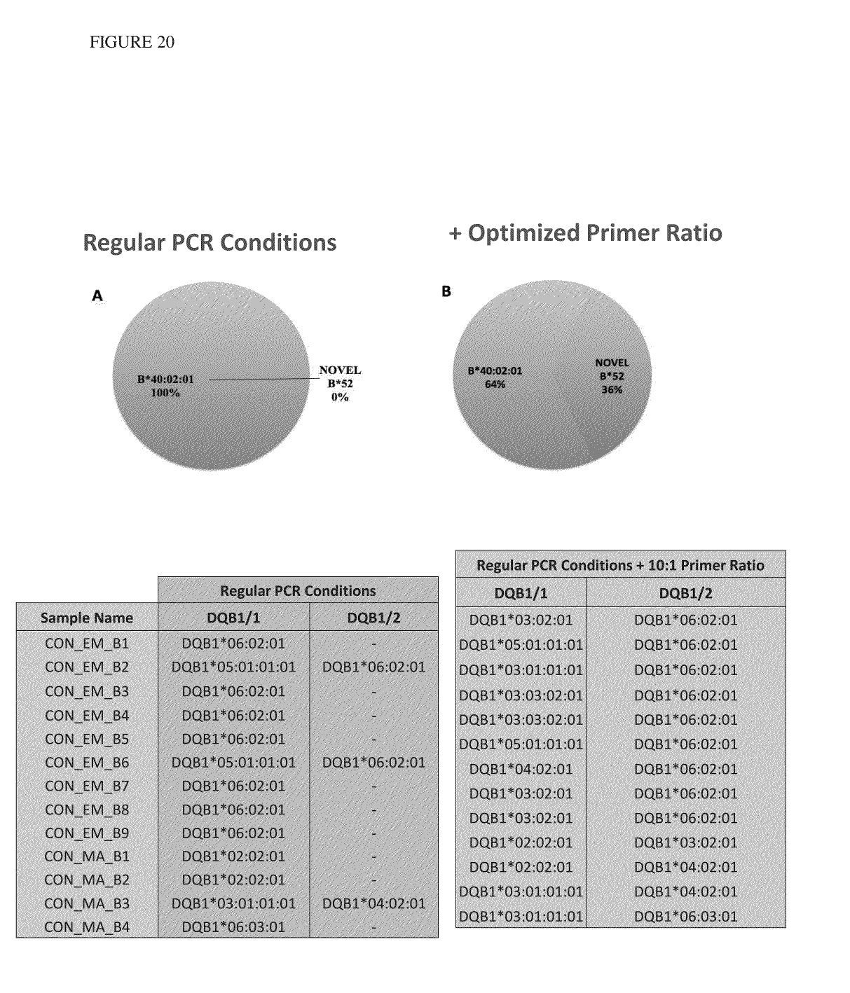

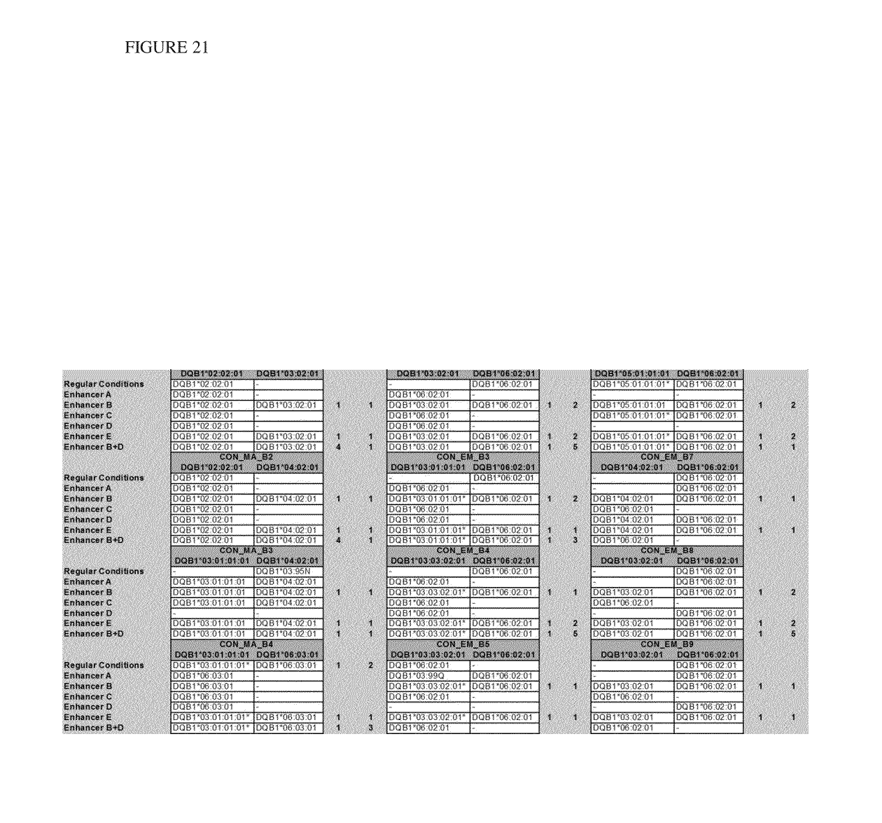

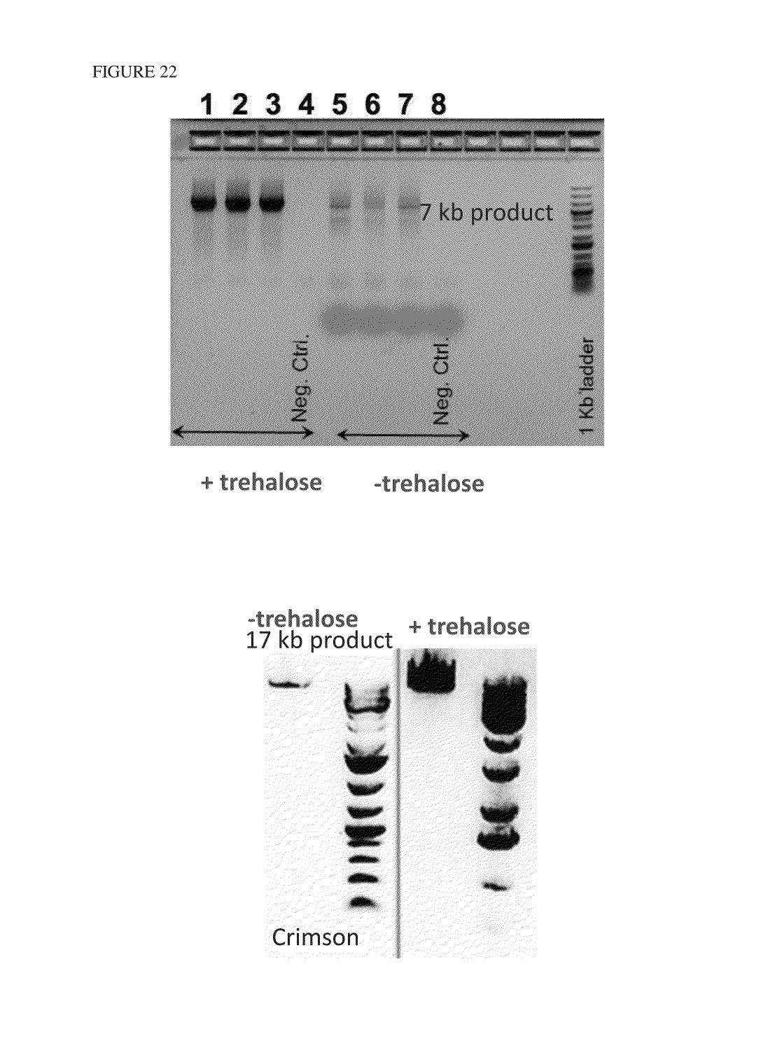

The amplification conditions can have an effect on reaction. Conditions such as primer design, polymerase, use of dNTP analogs (e.g. in primers and/or elongation), Tm, and chemical makeup (including ratios of components and/or the presence/absence of components in the reaction mix) can affect the reaction. In some embodiments, the specific combination of reaction conditions can affect the reaction. As disclosed herein, combinations of polymerase, primers, chemical make-up, and/or use of dNTP analogs affects the outcome. One affect can be allelic drop out. When a reaction condition reduces allelic drop out, it can be referred to as an “enhancer.” FIG. 20 shows exemplary data where allelic drop out is reduced for DQB1/1 and DQB1/2 when the primer ratio is optimized (e.g. a ratio of 10:1). FIG. 22 shows exemplary experimental results when using trehelose (e.g. trehelose helps reduce allelic drop out when a 7 kb fragment is amplified). FIG. 21 depicts exemplary data, showing the effect of several enhancers on allelic drop out. In some embodiments, more than one enhancer can be used (e.g. optimizing several reaction conditions: polymerase; nucleotide analogs used in primers; nucleotide analogs used in dNTP mix; addition of trehelose; primer ratio). In some embodiments multiple different combinations of enhancers can be used. In some embodiments, no enhancers may be used.

The polymerase used can have an effect on the reaction. FIG. 18 depicts exemplary results comparing the ability of different polymerases to amplify HLA-B. The polymerases compared in FIG. 18 include: Bioline Velocity DNA Polymerase, One Taq 2×MM DNA Polymerase, and Bioline Ranger DNA Polymerase. FIG. 19 depicts exemplary results comparing the ability of different enzymes to amplify HLA-A.

The amplification step can comprise the use of dNTP analogs. Exemplary dNTP analogs are disclosed herein. dNTP analogs can be used in primers, as disclosed herein. dNTP analogs can be used in addition to regular dNTPs during elongation. The ratio between a dNTP analog to its corresponding regular dNTP can have an effect on the reaction (e.g. in reduction of allelic dropout). In one non-limiting example, a ratio of about 2.7:1 about 2.8:1; about 2.9:1; about 3:1; about 3.1:1; about 3.2:1 between N⁴-methyl-2′-dCTP and 2′-dCTP can be preferred. In another non-limiting example, a ratio of about 2.7:1 about 2.8:1; about 2.9:1; about 3:1; about 3.1:1; about 3.2:1 between 7-deaza-dGTP and 2′-dGTP can be preferred.

The choice of polymerase and dNTP analog together can affect the reaction. For example, in some embodiments, it can be preferable to use Crimson LongAmp® Taq DNA Polymerase to amplify human genes or gene fragments (e.g. HLA) when dNTP analogs, such as (1-thio)-2′-dCTP, N⁴-methyl-2′-dCTP, 7-deaza-2′-dATP, (1-thio)-2′-dGTP and 7-deaza-dGTP, are used.

In some embodiments, addition of a chemical in the reaction mix can have an effect. In some instances, adding trehalose can improve the reliability and/or effectiveness of long-range PCR. For example, trehalose can reduce allelic drop-out.

Different polymerases can be used with different primers, and the addition of trehalose can have an effect on allelic drop out. In one non-limiting experimental example, trehalose had a negative effect on the Phire polymerase. In another non-limiting example, the addition of trehalose improved Crimson polymerase. For example, trehalose when used with Crimson, can reduce allelic drop out for HLA-B and DQ-B. In another example, trehalose when used with Crimson reduced allelic drop out for DQ-BA, -B; HLA-A, -B, -C; and DRB.

In one exemplary experiment, the following reaction conditions were used (see FIG. 22).

| | FIG. 22 reaction conditions | |
|---|---|---|
| | PCR conditions | PCR conditions |
| | Regular conditions | Enhancer conditions |
| Component | Final Concentration | Final Concentration |
| 5X Crimson LongAmp | | |
| Taq Reaction Buffer | 1X* | 1X* |
| 10 mM dNTPs | 300 μM | 300 μM |
| (2.5 mM each) | | |
| 10 μM Forward Primers | 0.04 μM (0.05-1 μM) | 0.04 μM (0.05-1 μM) |
| 10 μM Reverse Primers | 0.04 μM (0.05-1 μM) | 0.04 μM (0.05-1 μM) |
| Trehalose (1.5M) | No | .4M |
| Template DNA | 100 ng | 100 ng |
| Crimson LongAmp | | |
| Taq DNA Polymerase | 2.5 units/25 μl PCR | 2.5 units/25 μl PCR |
| Nuclease-free water up to | | |
| 25 ul | | |
| *1X Crimson buffer | | |
| 60 mM Tris-SO₄ | | |
| 20 mM (NH₄)₂SO₄ | | |
| 2 mM MgSO₄ | | |
| 3% Glycerol | | |
| 0.06% IGEPAL ® | | |
| CA-630 | | |

In another exemplary experiment, the following reaction conditions were compared (see FIG. 20).

| | PCR conditions FIG. 20 | |
|---|---|---|
| | PCR conditions | PCR conditions |
| | Regular conditions | Enhancer conditions |
| Component | Final Concentration | Final Concentration |
| 5X Crimson | | |
| LongAmp | | |
| Taq Reaction Buffer | 1X* | 1X* |
| 10 mM dNTPs | 300 μM | 300 μM |
| (2.5 mM each) | | |
| 10 μM Forward | 0.04 μM (0.05-1 μM) #1 | 0.04 μM (0.05-1 μM) #1 |
| Primers | | |
| 10 μM Reverse | 0.04 μM (0.05-1 μM) #2 | 0.04 μM (0.05-1 μM) #3 |
| Primers | | |
| Trehaose (1.5M) | .4M | .4M |
| Template DNA | 100 ng | 100 ng |
| Crimson LongAmp | | |
| Taq DNA | 2.5 units/25 μl PCR | 2.5 units/25 μl PCR |
| Polymerase | | |
| Nuclease-free water | | |
| up to 25 ul | | |
| *1X Crimson buffer | | |
| 60 mM TriS-SO₄ | | |
| 20 mM (NH₄)₂SO₄ | | |
| 2 mM MgSO₄ | | |
| 3% Glycerol | | |
| 0.06% IGEPAL ® | | |
| CA-630 | | |

In some instances, if the template is degraded or the quantity is not sufficient for robust amplification with long-range PCR, whole genome amplification of the template nucleic acid can be used to restore the condition for successful, robust long-range PCR.

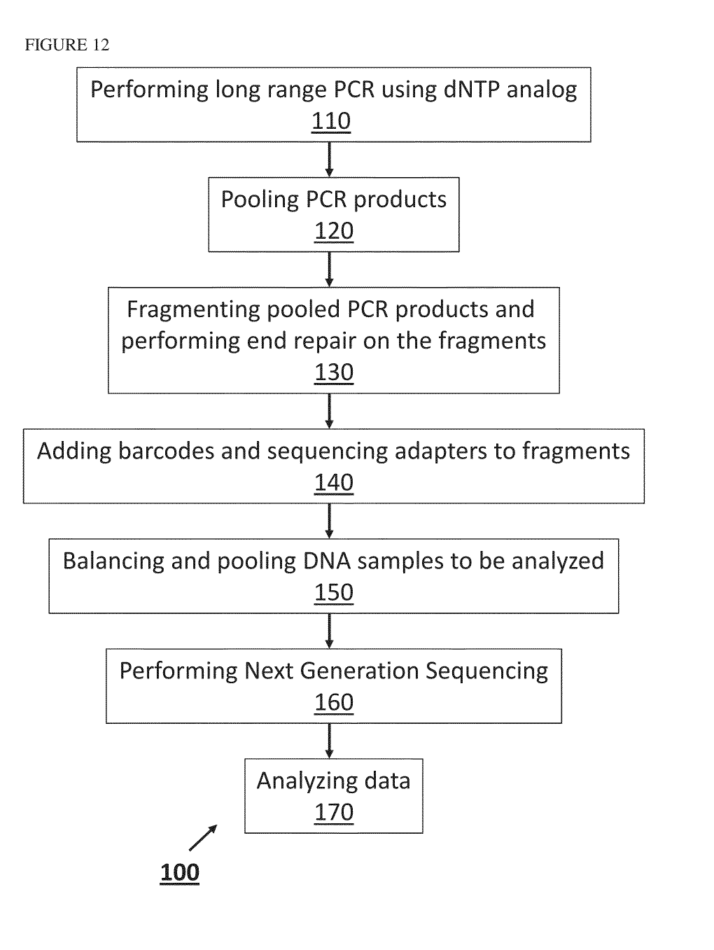

FIG. 12 depicts an exemplary sequence of steps which may be practiced in accordance with a method of the present disclosure. For example, after long range PCR amplification of step 110 is performed, PCR products of different genes may be quantified, balanced according to each allele relative to the other, and pooled in step 120. In some embodiments, equimolar amounts of the amplified gene products are pooled to ensure equal representation of each gene. A large number of samples can be typed in the same sequencing reaction, but the PCR yield is typically variable among different reactions. When the PCR products are pooled together without adjusting the relative amount among each gene in the same sequencing reaction, target genes with a higher PCR yield may have more sequencing reads, and those with a lower PCR yield may have fewer sequencing reads.

In some embodiments, quantification of PCR product amounts is determined, e.g. to ensure equal representation. One non-limiting way to quantify PCR products is by using the PicoGreen® dsDNA quantification assay (Life Technologies). However, one skilled in the art will understand that PCR products can be quantified by various methods. Depending on the amount of PCR products obtained for each gene or gene fragment or allele, a preferred ratio of PCR products of several genes and/or gene fragments and/or alleles, which can be pooled together, may be determined for the ensuing deep sequencing process. In one embodiment, an equimolar addition of all PCR products of selected genes and/or gene products and/or alleles may be pooled to ensure equal representation, or approximately equal representation of each gene/allele in the ensuing deep sequencing process. In another embodiment, a non-equimolar addition of some or all PCR products of selected genes and/or gene products and/or alleles may be pooled to ensure equal representation of each gene/allele in the ensuing deep sequencing process. Such a balancing step when pooling PCR samples may maximize the number of PCR samples we can multiplex per analytical sample in the deep sequencing process. For example, 4× the amount of HLA-DRB gene products and 0.5× of HLA-DPA gene products may be pooled to ensure better genotyping results for both genes in the same analytical sample for deep sequencing.

In some embodiments, an automatic amplicon balancing method may be applied. For example, after step 110, the concentration of each PCR product may be determined; then the size of amplicons, the number of target genes in each PCR reaction, and the concentration of amplicon in each well may be used to calculate the volume required for each amplicon in the pool. An amount for each gene in a sequencing sample may be determined by an automated amplicon balancing method.

Once PCR products are balanced and pooled in step 120, the pooled PCR products may be fragmented and end repaired in step 130. Either enzymatic or mechanical shearing may be employed in step 130. In one embodiment, fragmenting pooled PCR products was conducted by enzymatic shearing, for example, NEBNext® dsDNA Fragmentase in a time-dependent manner. In another embodiment, fragmentation is done by sonication. The desired length of DNA fragments may be, for example, about between 200-700 bp, about between 300-600 bp, about between 400-600 bp, about between 500-600 bp, and about between 400-500 bp. This desired length may be optimized for the specific sequencer used in the sequencing process. For example, a length of about between 500-600 bp may be preferred for an Illumina sequencer, HiSeq2000 instrument. If other sequence instruments are used, different size of DNA fragments might be selected. For example, one skilled in the art will recognize that the methods of this disclosure can be altered and optimized for various sequencing systems (e.g. Ion Torrent). Standard DNA end repair was performed by blunting and phosphorylating DNA ends of the fragments. For example, the Thermo Scientific Fast DNA End Repair Kit may be used for end repair in step 130 before the ensuing blunt-end ligation.

Step 140 adds barcodes and sequencing adapters to fragments after the end repair process is complete in step 130. In one embodiment, sequencing adapters were selected according to the sequencer machine which would be used in the sequencing process. In another embodiment, one pair of identical barcodes were ligated to both ends of one strand of DNA in the end-repaired DNA fragments; and a different pair of identical barcodes were ligated to both ends of the other strand of DNA in the same fragment. In one embodiment, each barcode differs in at least 2 positions to avoid sequencing error and cross contamination. In another embodiment, each barcode differs in at least 3 positions. In another embodiment, by ligating two pairs of distinct barcodes per sample, we have developed a strategy to provide greater evenness of coverage in sequencing and thereby increasing multiplicity of sequencing samples in a sequencing run. Each barcode may include a target specific identifier for the source of the genomic DNA and/or the gene so that a sequence, according to its barcode(s), may be assigned to the source sample and the gene from which the DNA sequence was obtained.

After the barcoding step 140, step 150 balances and pools barcoded DNA samples for the sequencing process. In one embodiment, multiple barcoded DNA samples were quantified using the method and balanced using the method. For example, 192 samples may be balanced and pooled into one individual lane for the Illumina sequencer. Another number of samples may be balanced and pooled for the same or different sequencer.

In one embodiment, the pooled DNA fragments were purified using AM-Pure XP beads (Beckman Coulter). In another embodiment, the purified DNA fragments may be selected according to their size using a Pippin Prep DNA size selection system (Sage Biosciences). For example, a size of about 400-700, about 500-700, about 500-600 may be used in this step.

In step 160, next generation sequencing was performed on balanced and pooled DNA fragments obtained in step 150. Sequence runs in some embodiments range from about 100 to about 500 nucleotides for each sample, and may be performed from each end of the ligated fragment.

Any appropriate sequencing method may be used in the context of the invention. Common methods include sequencing-by-synthesis, Sanger or gel-based sequencing, sequencing-by-hybridization, sequencing-by-ligation, or any other available method. Particularly preferred are high throughput sequencing methods. In some embodiments of the invention, the analysis uses pyrosequencing (e.g., massively parallel pyrosequencing) relying on the detection of pyrophosphate release on nucleotide incorporation, rather than chain termination with dideoxynucleotides, and as described by, for example, Ronaghi et al. (1998) Science 281:363; and Ronaghi et al. (1996) Analytical Biochemistry 242:84, herein specifically incorporated by reference. The pyrosequencing method is based on detecting the activity of DNA polymerase with another chemiluminescent enzyme. Essentially, the method allows sequencing of a single strand of DNA by synthesizing the complementary strand along it, one base pair at a time, and detected which base was actually added at each step. The template DNA is immobile and solutions of selected nucleotides are sequentially added and removed. Light is produced only when the nucleotide solution complements the first unpaired base of the template.

Sequencing platforms that can be used in the present disclosure include but are not limited to: pyrosequencing, sequencing-by-synthesis, single-molecule sequencing, nanopore sequencing, sequencing-by-ligation, or sequencing-by-hybridization. Preferred sequencing platforms are those commercially available from Illumina (RNA-Seq), Helicos (Digital Gene Expression or “DGE”), Ion torrent (Thermo Fisher). “Next generation” sequencing methods include, but are not limited to those commercialized by: 1) 454/Roche Lifesciences including but not limited to the methods and apparatus described in Margulies et al., Nature (2005) 437:376-380 (2005); and U.S. Pat. Nos. 7,244,559; 7,335,762; 7,211,390; 7,244,567; 7,264,929; 7,323,305; 2) Helicos BioSciences Corporation (Cambridge, Mass.) as described in U.S. application Ser. No. 11/167,046, and U.S. Pat. Nos. 7,501,245; 7,491,498; 7,276,720; and in U.S. Patent Application Publication Nos. US20090061439; US20080087826; US20060286566; US20060024711; US20060024678; US20080213770; and US20080103058; 3) Applied Biosystems (e.g. SOLiD sequencing); 4) Dover Systems (e.g., Polonator G.007 sequencing); 5) Illumina as described U.S. Pat. Nos. 5,750,341; 6,306,597; and 5,969,119; and 6) Pacific Biosciences as described in U.S. Pat. Nos. 7,462,452; 7,476,504; 7,405,281; 7,170,050; 7,462,468; 7,476,503; 7,315,019; 7,302,146; 7,313,308; and US Application Publication Nos. US20090029385; US20090068655; US20090024331; and US20080206764. All references are herein incorporated by reference. Such methods and apparatuses are provided here by way of example and are not intended to be limiting.

In one embodiment, the sequencing was performed using Illumina sequencer. In another embodiment, the sequencing was performed using an Ion Torrent sequencer.

In step 170, raw sequence data from the sequencing machine was received by and the machine readable code was transferred and read by a computer-based system for analysis. In one embodiment, the received raw data was de-multiplexed or deconvoluted according to their barcodes. Those sequences which had identical barcodes at both ends of one strand of DNA may be assigned to the same DNA fragment of interest and/or the same source sample according to the target specific barcode. In another embodiment, those sequences, wherein their two DNA strands have identical, paired barcodes on both ends, may be assigned to the same DNA fragment of interest and/or the same source sample according to the target specific barcode, and may be counted as one read. Each nucleotide of a target gene is read at least about 100 times, and may be read at least about 1000 times, or at least about 10,000 times.

In one embodiment, the received sequence data is deconvoluted and assigned to each sample, and to each gene using the target specific barcode for each fragment analyzed, if possible. Each nucleotide of a target gene can be read at least about 100 times, and may be read at least about 1000 times, or at least about 10,000 times. The process of deconvolution is the set of bioinformatics steps that take sequence reads for a particular gene, map it to its corresponding reference sequence. The novel computational algorithm “Chromatid Sequence Alignment” (CSA) can be applied for this purpose. The CSA algorithm was designed to use short DNA sequence fragments generated by high-throughput sequencing instruments. This algorithm efficiently clusters sequence fragments properly according to their origins and effectively reconstructs chromatid sequences. The output sequence from CSA algorithm consisting of consecutive nucleotides and covering an entire HLA gene provides the information to call haplotype of HLA loci, or any other similarly complex and polymorphic locus.

When sequence reads thus obtained are mapped onto a correct reference sequence, they form a continuous tiling pattern over the entire sequenced region. Reference sequences for the HLA region are known in the art and publicly available, for example including the IMGT-HLA database. When reads were mapped onto an incorrect reference sequence, they formed a staggered tiling pattern at some positions of the sequenced region or discontinued tiling patterns.

To quantify this difference between the two alignment patterns, the number of “central reads” for any given point is counted, where central reads are empirically defined as mapped reads for which the ratio between the length of the left arm and that of the right arm related to a particular point is between 0.5 and 2. The genotype-calling algorithm is based on the assumption that more reads are mapped to correct reference(s) than to incorrect reference(s). The minimum coverage of overall reads (MGOR) is computed; and the minimum coverage of central reads (MGGR) for each reference is computed. The MGGR values for 30 bases near intron/exon boundaries are ignored, as they are always zero, based on the definition of central reads and the cutoff length. References with an MGOR less than 20 and an MGGR less than 10 are eliminated, as they were unlikely to be correct.

From the remaining references, all possible combinations of either one reference (homozygous allele) or two references (heterozygous alleles) of the same gene are enumerated, and the number of distinct reads that mapped to each combination is counted. To compensate for a single reference (homozygous allele), the number of distinct reads is multiplied with an empirical value of 1.05 to avoid miscalls due to spurious alignments. The member(s) in the combination with maximum number of distinct reads is assigned as the genotype of that particular sample.

In another embodiment, the average MOOR of all reference sequences is at least 40, at least 60, at least 80, at least 100, at least 150, or at least 200. This central reads counting method may distinguish true HLA alleles from sequencing artifacts and thereby improve the reliability of HLA typing.

Optionally, to ensure that unmapped nucleotides outside aligned regions are taken into consideration, de novo assembly of mapped reads including their unmapped regions is performed. The mapped reads, including unmapped regions, are partitioned into tiled 40-base fragments with a one base offset. A directed weighted graph is built where each distinct fragment is represented as a node and two consecutive fragments of the same read are connected, and an edge between two nodes is weighted with the frequency of reads from the two connected nodes. A contig is constructed on the path with the maximum sum of weights. By comparing a contig with its corresponding reference sequence, differences between a contig built from reads and its closest reference can be identified.

After mapping the alignments may be parsed in the following order: a best-match filter, a mismatch filter, a length filter, and a paired-end filter. The best-match filter only keeps alignments with best bit-scores. The mismatch filter eliminated alignments containing either mismatches or gaps. The length filter deletes alignments shorter than 50 bases in length if their corresponding exons were longer than 50 bases. It also removed any alignments shorter than their corresponding exons if those were less than 50 bases in length. Finally, the paired-end filter removes alignments in which references were mapped to only one end of a paired-end read, while at least one reference was mapped to both ends of the paired-end read. In one embodiment, a consensus sequence may be deduced from analyzing mixed consensus sequences assigned to the same DNA fragment, gene or sample source.

The result is a set of sequences assigned to specific alleles for the HLA genes of interest. In one embodiment, the genotype of two alleles for each of HLA-A, HLA-B, HLA-C and HLA-DRB1 can be obtained. In another embodiment, the genotype of two alleles for each of HLA-A, HLA-B, HLA-C, HLA-DQA, and HLA-DQB may be obtained. In still another embodiment, class II genes of HLA-DRB, HLA-DQA, and HLA-DQB are usually inherited in one block. This sequence information thus provided may be used to diagnose a condition; for tissue matching; blood typing; and the like.

In one embodiment, in silico reference database filling may be performed on a computer-based system. In particular, confirmed consensus sequences are used to build a reference database for genes or alleles for a sample or samples. In silico reference database filling is based on the fact that new HLA genes are derived from closely related HLA genes through either mutation, deletion, insertion, gene shuffling et al. Therefore, genes sharing the same exon are likely to share neighboring introns, vice versa.

In another embodiment, in silico reference database validation may be performed on a computer-base system. Among reference sequences, newly called genotypes, and deep-sequencing data, deep sequencing data are less likely to be erroneous. In the validation process, the newly derived reference sequences will be compared to deep sequencing data the same way as the regular genotype calling. If the derived reference sequence is correct, the CSA algorithm will be able to verify that.

In still another embodiment, a number of new sequences obtained from NGS may be used to run a combined validation against the corresponding sequence in the reference database.

In one embodiment, bench verification via Sanger may be performed to validate a particular sequence in the reference database.

The CSA genotyping algorithm, in silico reference sequence database filling and consensus sequence calling and validation are formed into an integrated system (i.e. the acronym GSV can be used to refer to the process; e.g. Genotyping algorithm, in Silico and Validation). In the GCV system, Next Generation Sequencing data can be used as a reliable source of information.

In still another embodiment, a self-learning flagging system may be developed for HLA genotyping, wherein description.

The goal of the above in silico analysis is to build and refine a reference database which reflects and store reliable genetic information.

Also provided herein are software products tangibly embodied in a machine-readable medium, the software product comprising instructions operable to cause one or more data processing apparatus to perform operations comprising: a) clustering sequence data from a plurality of reads to generate a contig as described above; and b) providing an analysis output on said sequence data.

More specifically, a software product may comprise instructions for one or more of the following modules, e.g. to align sequence reads to reference sequences, to filter out incorrect alignments, to filter out unlikely reference candidates, to enumerate combinations of candidate alleles, to count the number of reads mapped to each combination of candidate alleles, to call genotype for each allele, and/or to derive the consensus sequence for each called allele. Each module may comprise one or more of the following:

Alignment of Sequences to a Reference Sequence.

i. reference sequences can be aligned to a database; ii. the number of central reads can be counted; iii. the minimum coverage of overall reads can be computed; iv. the minimum coverage of central reads for each reference sequence can be computed; v. combinations of all or substantially all combinations of homozygous alleles or heterozygous alleles of the same gene may be determined and distinct reads that map to each combination can be determined; and vi. The genotype can be assigned to the combination with maximum number of distinct reads.

Perform De Novo Assembly of Reads Including Unmapped Regions Outside of Reference Sequences.

i. reads can be partitioned, including unmapped regions, into short tiled fractions, e.g. tiles of from about 30, about 40, about 50 bases; with a one base offset. ii. a directed weighted graph can be built, wherein each distinct fragment can be represented as a node, wherein two consecutive fragments of the same read can be connected, and an edge between two nodes can be weighted with the frequency of reads from the two connected nodes; iii. a contig can be constructed on the path with the maximum sum of weights; and iv. the contig can be compared with its corresponding closest reference sequence.

Parse Alignments.

i. Filter to keep only alignments with best bit-scores. ii. Eliminate alignments containing either mismatches or gaps. iii. Delete alignments shorter than 50 bases in length if their corresponding exons were longer than 50 bases, and remove any alignments shorter than their corresponding exons if those were less than 50 bases in length. iv. Remove alignments in which references were mapped to only one end of a paired-end read, while at least one reference was mapped to both ends of the paired-end read.

In Silico Reference Database Filling.

Step 1 filling: for any pair of alleles of the same gene, compute the similarity score for each corresponding components (either exon or intron). The gapped component (either exon or intron) of allele Y is filled with the complete component from allele X if neighboring component of allele Y is most similar to the corresponding component of X. Step 2 validation: for any filled reference sequence (e.g. Y) will be checked against NGS data from a sample which is known has allele Y. The filled reference is put into reference database and will be checked whether it can be called by CSA algorithm.

Mapping-Assembling Consensus Calling.

Step 1 mapping. map sequencing reads onto all known genomic reference sequences including those from pseudogenes. Step 2 filtering: a. keep alignment with highest score for each read. b. for a pair-end read, if there is a reference sequence where both ends can be mapped to a reference sequence, then keep those alignments with both ends mapped to the same reference sequence. Step 3 build consensus. a. through read-reference alignments and reference-reference alignments, mapped reads are re-positioned to a universal coordinates for each gene. b. collapse alignment of each column to A, C, G and T and their corresponding frequency. c. keep bases of each column beyond frequency cutoff.

An Integrated System:

Among the four components: NGS data, CSA algorithm, consensus construction, and reference database filling and validation, NGS data is the most reliable information. The combination of NGS data and the CSA algorithm is used to validate filled reference sequences. In addition, the genotypes called by CSA algorithm and the consensus build by mapping-assembling algorithm are checked against each other: Case 1. Both alleles called by the CSA algorithm are complete. The polymorphic sites can be derived from the called allele reference sequences. The consistence between derived consensus sequences and the mapping-assembling consensus sequences will be used to calibrate the accuracy of genotype results. Case 2. If only one allele called is complete and the other one is partial. Those polymorphic sites derived from references are checked with the mapping-assembling consensus sequences. In addition, by subtracting the complete allele reference from the mapping-assembling consensus, the partial reference can be extended to be complete. The newly derived sequence for the partial reference will be put into reference database and checked whether it can be called by the CSA algorithm. Case 3. If both alleles called are incomplete. Those polymorphic sites derived from references are checked with the mapping-assembling consensus sequences. The mapping-assembling consensus is put into reference database and checked whether they can be called by the CSA algorithm. Case 4. If a novel allele in a sample, the mapping-assembling consensus will be checked with the known allele. The newly called reference will be put into reference database and checked whether it can be called by the CSA algorithm.

To further improve the accuracy of HLA genotype algorithm, a flagging system is implemented based on public information and pattern learned from results generated by this algorithm. In the flagging system, the linkage disequilibrium between different genes and sequencing depth et al are used to calibrated the reliability of genotypes.

Software products disclosed herein are software products tangibly embodied in a machine-readable medium, the software product comprising instructions operable to cause one or more data processing apparatus to perform operations comprising: storing sequence data and clustering the reads to a chromatid.

Information provided to an individual or for cataloging purposes. The HLA genotype results and databases thereof may be provided in a variety of media to facilitate their use. “Media” refers to a manufacture that contains the HLA genotype information of the present invention. The databases of the present invention can be recorded on computer readable media, e.g. any medium that can be read and accessed directly by a computer. Such media include, but are not limited to: magnetic storage media, such as floppy discs, hard disc storage medium, and magnetic tape; optical storage media such as CD-ROM; electrical storage media such as RAM and ROM; and hybrids of these categories such as magnetic/optical storage media. One of skill in the art can readily appreciate how any of the presently known computer readable mediums can be used to create a manufacture comprising a recording of the present database information. “Recorded” refers to a process for storing information on computer readable medium, using any such methods as known in the art. Any convenient data storage structure may be chosen, based on the means used to access the stored information. A variety of data processor programs and formats can be used for storage, e.g. word processing text file, database format, etc.

As used herein, “a computer-based system” refers to the hardware means, software means, and data storage means used to analyze the information of the present invention. The minimum hardware of the computer-based systems of the present invention comprises a central processing unit (CPU), input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture.

A variety of structural formats for the input and output means can be used to input and output the information in the computer-based systems of the present invention.

The deconvolution and chromatid sequence assignment analysis, e.g. one or more of the modules to align sequences to a reference sequence, to de novo assemble reads into a contig; and to parse the resulting alignments to provide the best match result for a genotype of each allele, may be implemented in hardware or software, or a combination of both. In one embodiment of the invention, a machine-readable storage medium is provided, the medium comprising a data storage material encoded with machine readable data which, when using a machine programmed with instructions for using said data, is capable of displaying any of the datasets and data comparisons of this invention. In some embodiments, the invention is implemented in computer programs executing on programmable computers, comprising a processor, a data storage system (including volatile and non-volatile memory and/or storage elements), at least one input device, and at least one output device. Program code is applied to input data to perform the functions described above and generate output information. The output information is applied to one or more output devices, in known fashion. The computer may be, for example, a personal computer, microcomputer, or workstation of conventional design.

Each program can be implemented in a high level procedural or object oriented programming language to communicate with a computer system. However, the programs can be implemented in assembly or machine language, if desired. In any case, the language may be a compiled or interpreted language. Each such computer program can be stored on a storage media or device (e.g., ROM or magnetic diskette) readable by a general or special purpose programmable computer, for configuring and operating the computer when the storage media or device is read by the computer to perform the procedures described herein. The system may also be considered to be implemented as a computer-readable storage medium, configured with a computer program, where the storage medium so configured causes a computer to operate in a specific and predefined manner to perform the functions described herein. A variety of structural formats for the input and output means can be used to input and output the information in the computer-based systems of the present invention.

Further provided herein is a method of storing and/or transmitting, via computer, sequence, and other, data collected by the methods disclosed herein. Any computer or computer accessory including, but not limited to software and storage devices, can be utilized to practice the present invention. Sequence or other data (e.g., HLA genotype analysis results), can be input into a computer by a user either directly or indirectly. Additionally, any of the devices which can be used to sequence DNA or analyze DNA or analyze HLA genotype data can be linked to a computer, such that the data is transferred to a computer and/or computer-compatible storage device. Data can be stored on a computer or suitable storage device (e.g., CD). Data can also be sent from a computer to another computer or data collection point via methods well known in the art (e.g., the internet, ground mail, air mail). Thus, data collected by the methods described herein can be collected at any point or geographical location and sent to any other geographical location.

Referring now to the drawings and with specific reference to FIG. 12, a method of high throughput genotyping according to the present disclosure is shown in detail. More specifically, one embodiment of the method of the present disclosure, indicated generally by the numeral 100, may sequentially include: performing long rang PCR reaction using dNTP analog (110); pooling PCR products (120); fragmenting pooled PCR products and performing end repair on the obtained fragments (130); adding barcodes and sequencing adapters to fragments (140); balancing and pooling DNA samples to be analyzed (150); performing Next Generation Sequencing on the DNA samples (160); and analyzing sequencing data obtained to complete genotyping

### Reagents and Kits

Also provided are reagents and kits thereof for practicing one or more of the above-described methods. The subject reagents and kits thereof may vary greatly. Reagents of interest include reagents specifically designed for use in production of the above described HLA genotype analysis. For example, reagents can include primer sets for PCR amplification and/or for high throughput sequencing. In some embodiments, a kit is provided comprising a set of primers suitable for amplification of the one or more genes of the HLA locus, e.g. the class I genes: HLA-A, HLA-B, HLA-C; the Class II gene HLA-DQA and HLA-DQB, etc. The primers are optionally selected from those shown in Table 1.

The kits of the subject invention can include the above described gene specific primer collections. The kits can further include a software package for sequence analysis. The kit may include reagents employed in the various methods, such as primers (including primers containing dNTP analog(s)) for generating copies of target nucleic acids, dNTPs, dNTP analogs, and/or rNTPs, which may be either premixed or separate, one or more uniquely labeled dNTPs and/or rNTPs, such as biotinylated or Cy3 or Cy5 tagged dNTPs, gold or silver particles with different scattering spectra, or other post synthesis labeling reagent, such as chemically active derivatives of fluorescent dyes, enzymes, such as reverse transcriptases, DNA polymerases, RNA polymerases, and the like, various buffer mediums, e.g. hybridization and washing buffers, prefabricated probe arrays, labeled probe purification reagents and components, like spin columns, etc., signal generation and detection reagents, e.g. streptavidin-alkaline phosphatase conjugate, chemifluorescent or chemiluminescent substrate, and the like.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed, site. Any convenient means may be present in the kits.

The above-described analytical methods may be embodied as a program of instructions executable by computer to perform the different aspects of the invention. Any of the techniques described above may be performed by means of software components loaded into a computer or other information appliance or digital device. When so enabled, the computer, appliance or device may then perform the above-described techniques to assist the analysis of sets of values associated with a plurality of genes in the manner described above, or for comparing such associated values. The software component may be loaded from a fixed media or accessed through a communication medium such as the internet or other type of computer network. The above features are embodied in one or more computer programs may be performed by one or more computers running such programs.

Software products (or components) may be tangibly embodied in a machine-readable medium, and comprise instructions operable to cause one or more data processing apparatus to perform operations comprising: a) clustering sequence data from a plurality of immunological receptors or fragments thereof; and b) providing a statistical analysis output on said sequence data. Also provided herein are software products (or components) tangibly embodied in a machine-readable medium, and that comprise instructions operable to cause one or more data processing apparatus to perform operations comprising: storing and analyzing sequence data.

### EXAMPLES

The following examples are offered by way of illustration and not by way of limitation.

### Example 1

### Accurate Determination of Haplotype of HLA Loci with Ultra-Deep, Shot-Gun Sequencing

Human leukocyte antigen (HLA) genes are the most polymorphic in the human genome. They play a pivotal role in the immune response and have been implicated in numerous human pathologies, especially autoimmunity and infectious diseases. Despite their importance, however, they are rarely characterized comprehensively because of the prohibitive cost of standard technologies and the technical challenges of accurately discriminating between these highly-related genes and their many alleles. Here we demonstrate a novel, high resolution, and cost-effective methodology to type HLA genes by sequencing, that combines the advantage of long-range amplification and the power of high-throughput sequencing platforms. We calibrated our method using 40 reference cell lines for HLA-A, -B, -C, and -DRB1 genes with an overall concordance of 99% (226 out of 229 alleles), and the 3 discordant alleles were subsequently re-analyzed to confirm our results. We also typed 59 clinical samples in one lane of an Illumina HiSeq2000 instrument and identified three novel alleles with insertions and deletions. We have further demonstrated the utility of this method in a clinical setting by typing five clinical samples in an Illumina MiSeq instrument with a five-day turnaround. The data analysis included allele calls that were made virtually by the software with no operator evaluation. In most instances, the fourth field data contained no previous information. This yielded stronger haplotype expectations than expected and several common allele subtypes were distinguished at the fourth field. Specific allele associations became apparent without any assumptions made. These studies show the robustness and comprehensive coverage provided by the typing system.

Overall, this technology has the capacity to deliver low-cost, high-throughput, and accurate HLA typing by multiplexing thousands of samples in a single sequencing run. Furthermore, this approach and can also be extended to include other polymorphic genes that are important in immune responses, or other important functions. Other advantages of this method include the use of clonal template amplification in vitro to eliminate the problem of sequencing heterozygous DNA, a sufficiently long read length (300+bp) to cover entire exons in phase, increased sequence coverage of HLA genes, capability to multiplex patient specimens, and the potential to complete run and data analysis within one week.

Human leukocyte antigen (HLA) genes encode cell-surface proteins that bind and display fragments of antigens to T lymphocytes. This helps to initiate the adaptive immune response in higher vertebrates and thus is critical to the detection and identification of invading microorganisms. Six of the HLA genes (HLA-A, -B, -C, -DQA1, -DQB1 and -DRB1) are extremely polymorphic and constitute the most important set of markers for matching patients and donors for bone marrow transplantation. For example, assume that in bone marrow transplantation a donor carries an expressed allele. If, in fact, the allele is not expressed this could result in a mismatch in the graft-versus host direction. If null alleles are not expressed, the assumption that a patient carries an expressed allele, when in fact the allele is not expressed, results in a mismatch in the rejection direction.

In another bone marrow null allele example, a novel A-locus allele was identified by sequence based typing of a bone marrow donor whose HLA typing results showed some inconsistencies. The donor was initially typed by CDC (complement dependent cytotoxicity) and forward PCR-SSOP utilizing commercial primers and probes; these results showed only HLA-A3 or 03XX allele. This donor was then selected for further testing as in a bone marrow donor search. The confirmatory typing showed A*03XX and 23XX by SSP. It could be argued that the nucleotide substitution alters the intron-3 splicing site since the canonical motif GGT (exon-G-GT-intron) present in the exon/intron junction of exons 1 to 5 HLA-A and B and most C-alleles. The nucleotide substitution in the novel allele would then lead to the production of a mRNA with an anomalous sequence. The RNA could be spliced downstream of the normal exon-3/intron-3 splicing site; for example the sequence GGT is found in nucleotides 40-42 of intron 3 and could therefore serve as an alternative splicing site. If this was the case then the sequence of exon-3 would be elongated and an in-phase termination codon would be found (TGA at codon 192 if the elongated exon was generated)

Specific HLA alleles have also been found to be associated with a number of autoimmune diseases, such as multiple sclerosis, narcolepsy, celiac disease, rheumatoid arthritis and type I diabetes. Alleles have also been noted to be protective in infectious diseases such as HIV, and numerous animal studies have shown that these genes are often the major contributors to disease susceptibility or resistance.

HLA genes are among the most polymorphic in the human genome, and the changes in sequence affect the specificity of antigen presentation and histocompatibility in transplantation. A variety of methodologies have been developed for HLA typing at the protein and nucleic acid level. While earlier HLA typing methods distinguished HLA antigens, modern methods such as sequence-based typing (SBT) determine the nucleotide sequences of HLA genes for higher resolution. However, due to cost and time constraints, HLA sequencing technologies have traditionally focused on the most polymorphic regions encoding the peptide-binding groove that binds to HLA antigens, i.e. exons 2 and 3 for the class I genes, and exon 2 for class II genes. Although the polymorphic regions of HLA genes predominantly cluster within these exons, an increasing number of alleles display polymorphisms in other exons and introns as well. Therefore, typing ambiguities can result from two or more alleles sharing identical sequences in the targeted exons, but differing in the exons that are not sequenced. Resolving these ambiguities is costly and labor-intensive, which makes current SBT methods unsuitable for studies involving even a moderately large group of samples.

Next generation typing systems, such as the one described here, offer significantly better accuracy compared to conventional methods. These new typing systems substantially enhanced allele resolution and dramatically improved combination resolution. Further, they offer the highest coverage of all major HLA gene regions: HLA-A, -B, -C, all exons, introns and 5′ and 3′ UTR; HLA-DPA1 and -DQA1, all exons and introns; HLA-DQB1, all exons and introns except intron 5 and exon 6; HLA-DRB1, 3/4/5, all exons and introns except part of introns 1&5 and exon 6; HLA-DPB1, all exons and introns except exons 1&5 and introns 1&4; and limited allele ambiguities (e.g. DPB1*13:01:01/DPB1*107:01 (ex1)).

Next generation typing systems also offer the ability to obtain sequence phase information. Paired-end sequencing results in phasing of approximately 600 base segments and no genotype ambiguities with the exception of the genotypes DPB1*04:01:01:01, 04:02:01:01, vs. DPB1*105:01, 126:01. These next generation typing systems could offer reduced time to identify donor-recipient match, the highest resolution and zero ambiguity, require no secondary testing, and allow physicians to immediately identify optimal matches. Next generation typing systems may detect novel HLA alleles, a capability that is limited or not possible with any gold-standard typing method.

Here we demonstrate a novel method targeting a contiguous segment of each of four polymorphic HLA genes (HLA-A, -B, -C and -DRB1), which define the minimal requirements for HLA matching for allogeneic hematopoietic stem cell transplantation (HSCT). Each HLA gene was amplified from genomic DNA in a single long-range polymerase chain reaction spanning the majority of the coding regions and covering most known polymorphic sites. This approach has several advantages. First, more polymorphic sites are sequenced to provide genotyping information of higher definition and the physical linkage between exons can be determined to resolve combination ambiguity. Second, long-range PCR primers can be placed in less polymorphic regions, allowing for improved resolution of genetic differences. Third, exons of the same gene can be amplified in one fragment, thereby decreasing coverage variability. We calibrated this typing method on HLA-A, -B, -C, and -DRB1 genes using 40 reference cell-line samples in the SP reference panel provided by the International Histocompatibility Working Group (IHWG, www.ihwg.org) The overall concordance rate of 99% with previous results and verification of our HLA typing results in the 3 discordant alleles by an independent sequencing technology demonstrate that this low-cost, high-throughput HLA typing protocol provides a high level of reliability. In addition, we tested our method on 59 clinical samples and found three new alleles (two short insertions and one single-base deletion), further illustrating the ability of this method to discover novel alleles. New alleles can also be found through Sanger Heterozygous SBT sequencing. We can apply other methods to identify which allele has the new allele (either null or novel) and serology may be the second method to assess expression. Alternatively, another family member sharing one haplotype with the proband may be tested. Isolation of the segment of the novel change (PCR or DNA or RNA strand capture) is called Novel polymorphism (NGS) and may be immediately mapped to one allele.

We designed PCR primers for each gene such that the most polymorphic exons and the intervening sequences could be amplified as a single product. For the class I genes HLAA, -B, and -C, primer sequences were selected to amplify the first seven exons. For HLADRB1, we designed primers to capture exons 2-5 and to avoid amplifying a large (approx. 8 kb) intron between exon 1 and exon 2. Equimolar amounts of the four HLA gene products were pooled to ensure equal representation of each gene and ligated together to minimize bias in the representation of the ends of the amplified fragments. These ligated products were then randomly sheared to an average fragment size of 300-350 bp and prepared for Illumina sequencing, after the addition of unique barcodes to identify the source of genomic DNA for each sample, using encoded sequencing adaptors. Each sequencing adaptor had a seven base barcode between the sequencing primer and the start of the DNA fragment being ligated. The barcodes were designed such that at least three bases differed between any two barcodes. Samples sequenced in the same lane were pooled together in equimolar amounts. The sequences of 150 bases from both ends of each fragment for cell-line samples were determined using the Illumina GAIIx sequencing platform. For clinical samples, the sequences of 100 and 150 bases from both ends of each fragment were determined with the Illumina HiSeq2000 and MiSeq platforms, respectively. For GAIIx sequence reads (counting each paired-end read as 2 independent reads), 91.8% of the sequence reads were parsed and separated according to their barcode tags.

After stripping the barcode tags, 95.5% (approximately 54 million sequence reads) were aligned to genomic reference sequences from the IMGT-HLA database with the NCBI BLASTN program, resulting in an average of 10,600 reads per position (coverage), which was estimated based on the number of reads mapped to genomic reference sequences without filtering. For clinical samples, 97.7% of the sequence reads from the HiSeq2000 instrument were parsed and separated according to their barcode tags. After stripping the barcode tags, 96.7% (around 152 million sequence reads) were aligned to genomic references, resulting in an estimated average of 10,000 reads per position.

Normalize and Pool PCR Products. In the methods of the invention, all major HLA genes including HLA-A, HLA-B, HLA-C, HLA-DPA1, HLA-DPB1, HLA-DQA1, HLA-DQB1, HLA-DRB1, HLA-DRB3, HLA-DRB4, HLA-DRB5 can be typed together for each sample (subject). Some genes are amplified in independent PCR reactions and pooled together in sequencing reaction. In addition, ten to thousand samples can be typed in the same sequencing reaction. However, the PCR yield is typically variable among different reactions. When the PCR products are pooled together without adjusting the relative amount among each genes in the same sequencing reaction, target genes with a higher PCR yield will have more sequencing reads, and those with a lower PCR yield will have fewer sequencing reads.

The HLA genotype calling method described below requires a minimum number of reads for each target gene to make a reliable calling. Therefore, the imbalance of sequencing reads directly impacts how many targets of each sample, and how many samples can be pooled together and reliably typed in one sequencing reaction.

To address this issue, an automatic amplicon balancing strategy was developed. After a PCR reaction, the PicoGreen assay is used to find the concentration of the PCR product of each. Taking into account the size of amplicons, the number of target genes in each reaction, and the concentration of each well in consideration, it is calculated what volume of each sample in the pool is required to make each product equimolar. All procedures are carried out automatically in a liquid handling system.

Classical HLA genotype assignment. Although genomic DNA was amplified and sequenced in our current approach, the standard genotype-calling algorithm relies mainly on the alignment to cDNA references from the IMGT-HLA database due to the lack of genomic reference sequences. Out of 6398 cDNA reference sequences for HLA-A, -B, -C and -DRB1 genes in the IMGT-HLA database released on Oct. 10, 2011, only 375 (5.8%) of them have genomic sequences. The IMGT-HLA database contains sequences of HLA genes, pseudogenes, and related genes, which allowed us to filter out sequences from pseudogenes or other non-classical HLA genes, such as HLA-, E, -F, -G, -H, -J, -K, -L, -V, -DRB2, -DRB3, -DRB4, DRB5, -DRB6, -DRB7, -DRB8, and -DRB9. After mapping, the alignments were parsed in the following order: a best-match filter, a mismatch filter, a length filter, and a paired-end filter. The best-match filter only kept alignments with best bit-scores. The mismatch filter eliminated alignments containing either mismatches or gaps. The length filter deleted alignments shorter than 50 bases in length if their corresponding exons were longer than 50 bases. It also removed any alignments shorter than their corresponding exons if those were less than 50 bases in length. Finally, the paired-end filter removed alignments in which references were mapped to only one end of a paired-end read, while at least one reference was mapped to both ends of the paired-end read.

HLA genes share extensive similarities with each other, and many pairs of alleles differ by only a single nucleotide; it is this extreme allelic diversity that has made definitive SBT difficult and subject to misinterpretation. For instance, due to the short read lengths generated using the Illumina platform, it is possible for the same read to map to multiple references. In this study, sequencing was performed in the paired-end format so that the combined specificity of paired-end reads could be used to minimize mis-assignment to an incorrect reference. Also, because of sequence similarities amongst different alleles, combinations of different pairs of alleles could result in a similar pattern of observed nucleotide sequence, based on the fortuitous mixture of sequences.

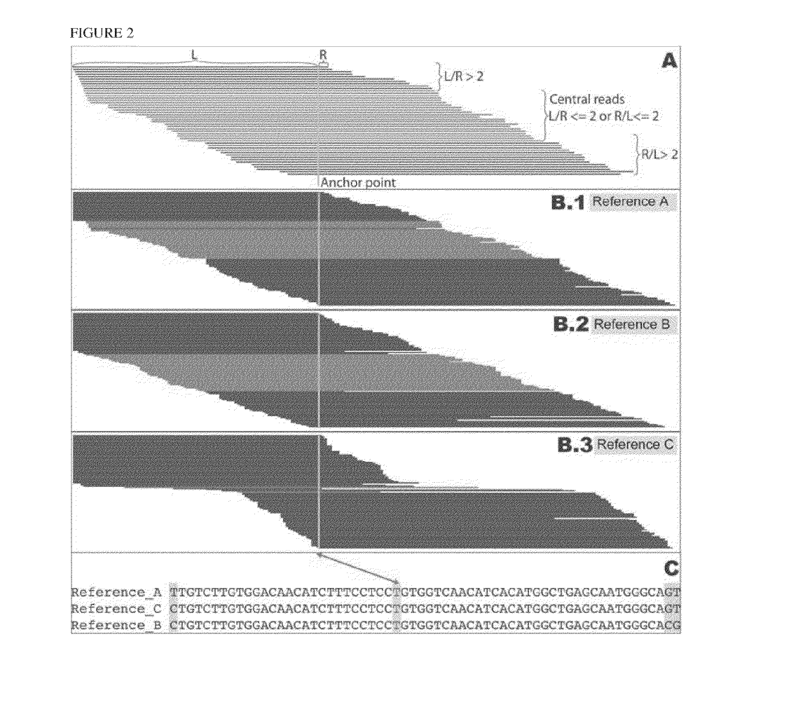

We noted that when reads were mapped onto a correct reference sequence, they formed a continuous tiling pattern over the entire sequenced region (FIGS. 2B.1 and 2B.2). When reads were mapped onto an incorrect reference sequence, they formed a staggered tiling pattern at some positions of the sequenced region (FIG. 2B.3). To quantify this difference between the two alignment patterns, we counted the number of “central reads” for any given point. Central reads (FIG. 2 A) were empirically defined as mapped reads for which the ratio between the length of the left arm and that of the right arm related to a particular point is between 0.5 and 2 (FIG. 2). The genotype-calling algorithm is based on the assumption that more reads are mapped to correct reference(s) than to incorrect reference(s). We could, in a brute-force manner, enumerate all possible combinations of references and count the number of mapped reads for each combination. However, due to the large number of possible combinations, this approach is very inefficient.

Therefore, we applied a heuristic approach to eliminate those implausible references first. We computed the minimum coverage of overall reads (MOOR) and the minimum coverage of central reads (MCCR) for each reference. We ignored the MCCR values for 30 bases near intron/exon boundaries, which were always zero, based on the definition of central reads and the cutoff length (FIG. 2). We eliminated the references with an MOOR less than 20 and an MCCR less than 10, as they were unlikely to be correct. From the remaining references, we enumerated all possible combinations of either one reference (homozygous allele) or two references (heterozygous alleles) of the same gene, and counted the number of distinct reads that mapped to each combination. To compensate for a single reference (homozygous allele), the number of distinct reads was multiplied with an empirical value of 1.05 to avoid miscalls due to spurious alignments. The member(s) in the combination with maximum number of distinct reads were assigned as the genotype of that particular sample. The aforementioned procedure only used the sequence information in the aligned region to do genotype calling. Such a process necessarily introduces bias in the interpretation, since it relies on existing reference data.

However, unmapped nucleotides outside aligned regions could also have important sequence information for new alleles. To ensure that they were taken into consideration, we implemented a program named EZ_assembler which carries out de novo assembly of mapped reads including their unmapped regions. Briefly, we partitioned the mapped reads, including unmapped regions, into tiled 40-base fragments with a one base offset. We built a directed weighted graph where each distinct fragment was represented as a node and two consecutive fragments of the same read were connected, and an edge between two nodes was weighted with the frequency of reads from the two connected nodes. A contig was constructed on the path with the maximum sum of weights. By comparing a contig with its corresponding reference sequence, we were able to identify differences between a contig built from reads and its closest reference. We applied the de novo assembly procedure for each candidate allele to verify the accuracy of the HLA typing, and to detect novel alleles.

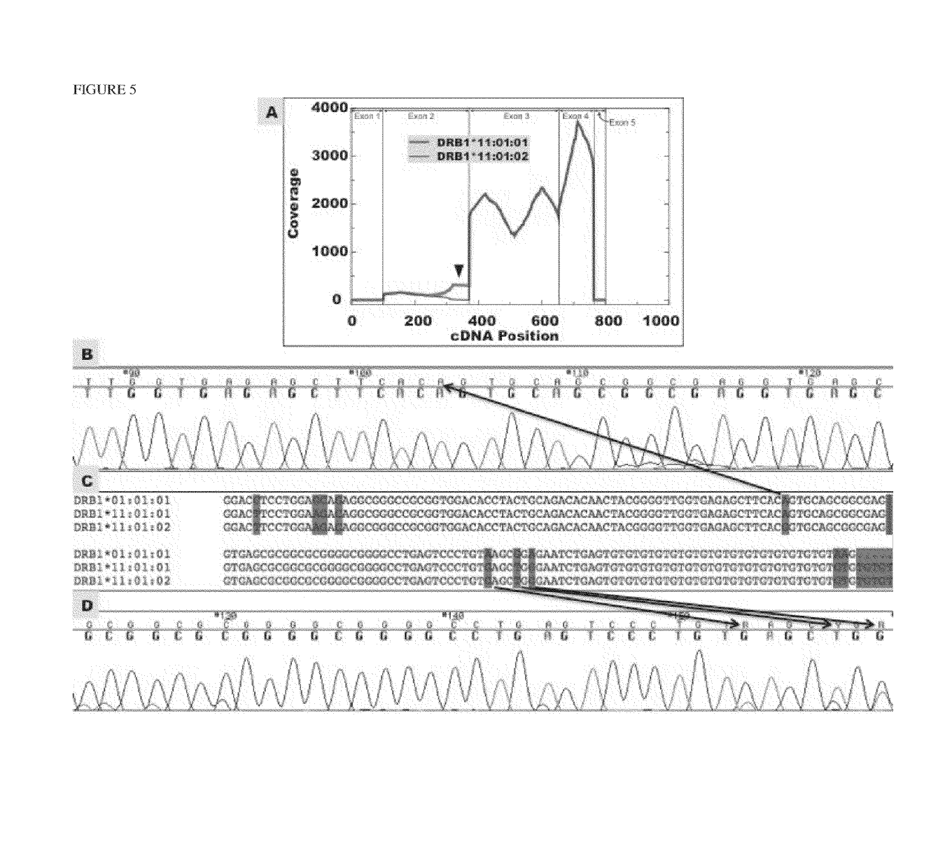

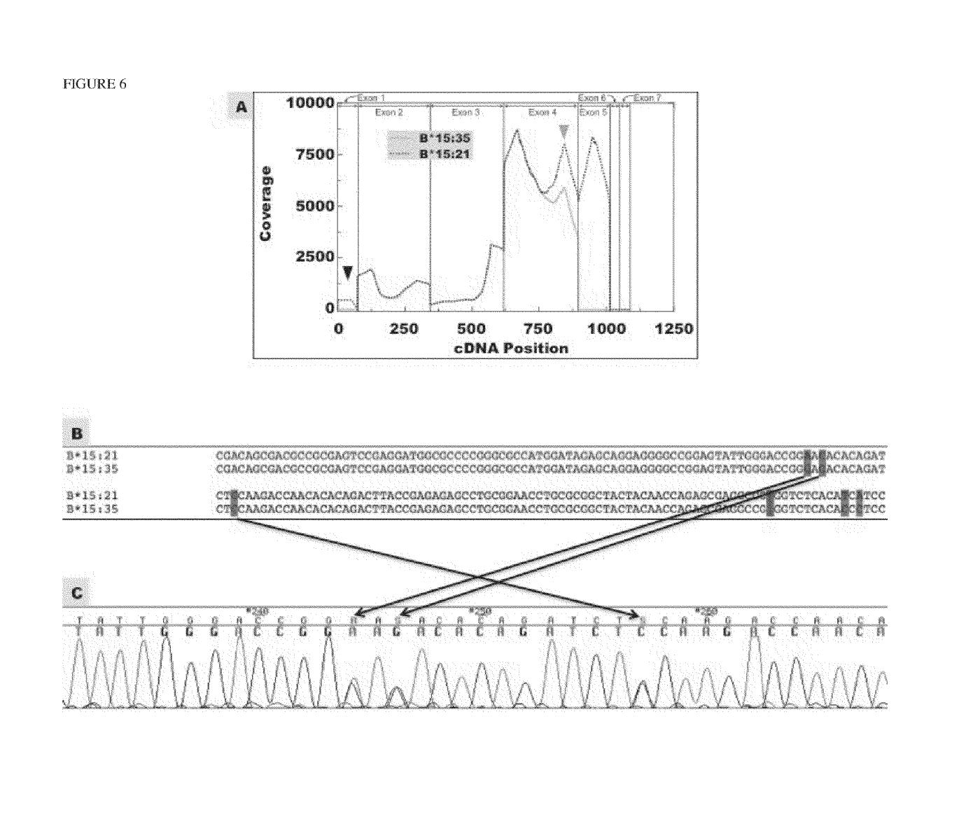

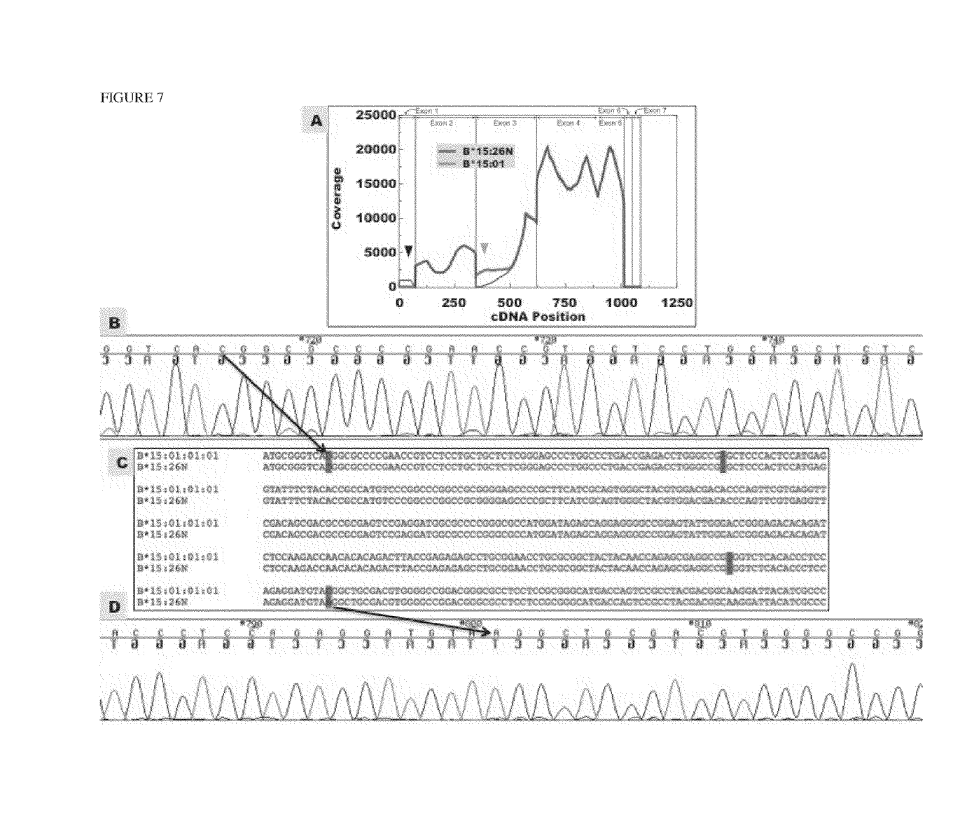

Genotyping four highly polymorphic HLA genes in 40 cell-lines. A total of 40 cell-line derived DNA samples of known HLA type were obtained from IHWG and sequenced at four loci (HLA-A, -B, -C, and -DRB1). We compared our predictions with the genotypes reported in the public database for those cell-lines. Out of 229 alleles from the 40 cell-lines typed for HLA-A, -B, -C, and -DRB1 loci, the concordance of our approach with previously determined HLA types was 99% (226/229). To further test the accuracy of our approach, we evaluated these discordant alleles by using an independent long-range PCR amplification, and sequenced the PCR products using Sanger sequencing. The HLA-DRB1 gene in the cell line FH11 (IHW09385) was previously reported as 01:01/11:01:02, which we found to be 01:01/11:01:01. One nucleotide, 12 bases upstream from the end of exon 2, differentiated HLA-DRB1*11:01:01 from HLA-DRB1*11:01:02. Sanger sequencing verified that the HLA-DRB1 gene of the cell-line FH11 is 01:01/11:01:01 (FIG. 5). The reference alleles listed for the HLA-B gene of the cell-line FH34 (IHW09415) are 15/15:21 and based on our sequencing data we are able to extend the resolution to 15:35/15:21. Our data showed that Illumina sequencing reads were aligned to both HLA-B*15:21/15:35 references continuously. HLA-B*15:21 and HLA-B*15:35 were different in 3 positions in exon 2, and 7 positions in exon 3. The Sanger sequencing chromatogram indicated the presence of a mixture in the corresponding positions at exon 2, matching the expected combination of HLA-B*15:21/15:35 (FIG. 6). The HLA-B gene of the cell-line ISH3 (IHW09369) was reported as homozygous for 15:26N in the IHWG cell-line database. Our Illumina sequencing reads mapped to exon 2, 3, 4, and 5, but not exon 1 of the HLA-B*15:26N reference. Instead, the reads mapped to exon1, 3, 4, and 5, but not exon 2 of the HLAB* 15:01:01:01 reference. There is no reference sequence available where the Illumina reads could tile continuously across the reference sequence. The Sanger sequencing data confirmed that ISH3 HLA-B allele had the exon 1 sequence as that of 15:01:01:01 and the sequence of exons 2, 3, 4, and 5 of 15:26N (FIG. 7). This suggests that either there is an error in the exon 1 region of B*15:26N reference sequence or that this represents yet another new B*15 null allele.

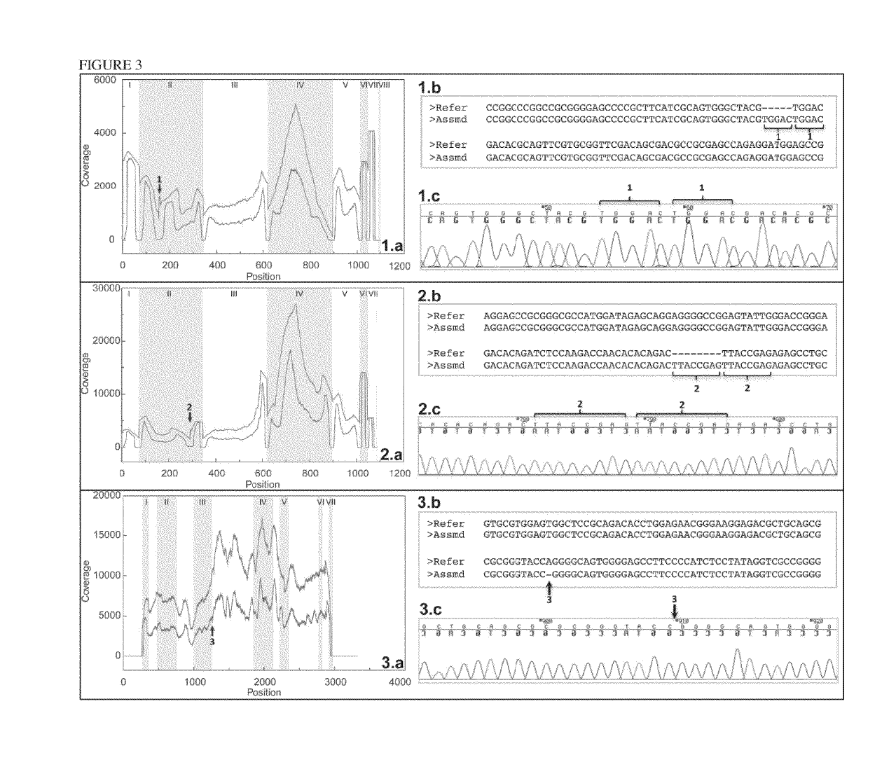

Genotyping four highly polymorphic HLA genes in 59 clinical samples. To test increased throughput using our approach, we pooled 59 clinical samples and typed HLA-A, -B, -C and -DRB1 in a single HiSeq2000 lane. Of these, 47 samples from an HLA disease association study were typed both by our novel methodology and an oligonucleotide hybridization assay. Even though the resolution of the probe-based assay was lower, the pairwise comparisons of possible genotypes showed overlap in at least one possible genotype for all loci in all samples. There were no allele dropouts in testing by either methodology. Twelve additional samples included specimens of HSCT patients or donors that presented less common or novel allele types (samples 48 to 59). In this group two samples with insertions of 5 and 8 exonic nucleotide insertions were concordantly typed by both classic Sanger sequencing and by the novel methodology described in the present study (FIGS. 3.1 and 3.2). The occurrence of these insertions shows a change in the reading frame with the occurrence of premature termination codons; therefore the corresponding mature HLA proteins of these alleles are not expressed on the cell surface (null). In conventional sequencing, both of heterozygous alleles are co-amplified and sequenced. However, when one of the alleles contains an insertion or deletion, it results in an off-phase heterozygous sequence and the read-out is cumbersome and laborious; in contrast, the read-out obtained by the novel methodology was straightforward.

The precise identification of the type of insertion/deletion in these novel alleles is of crucial importance in clinical histocompatibility practice. The allele containing the insertion or deletion may not be expressed because the reading frame may include changes in the amino acid sequence, resulting in the occurrence of premature termination codons, or it may have altered expression if the mutations are close to mRNA splicing sites (FIG. 3.3). If a mutation of this nature is overlooked, the evaluation of the HLA typing match between a patient and an unrelated donor could easily be incorrect. In the present study we identified the alleles B*40:01:02, A*23:17 and C*07:01:02; which are thought to be rare. But from the data presented here, it is likely that some of them may be the predominant allele of their group (B*40:01:02) or more common than previously thought.

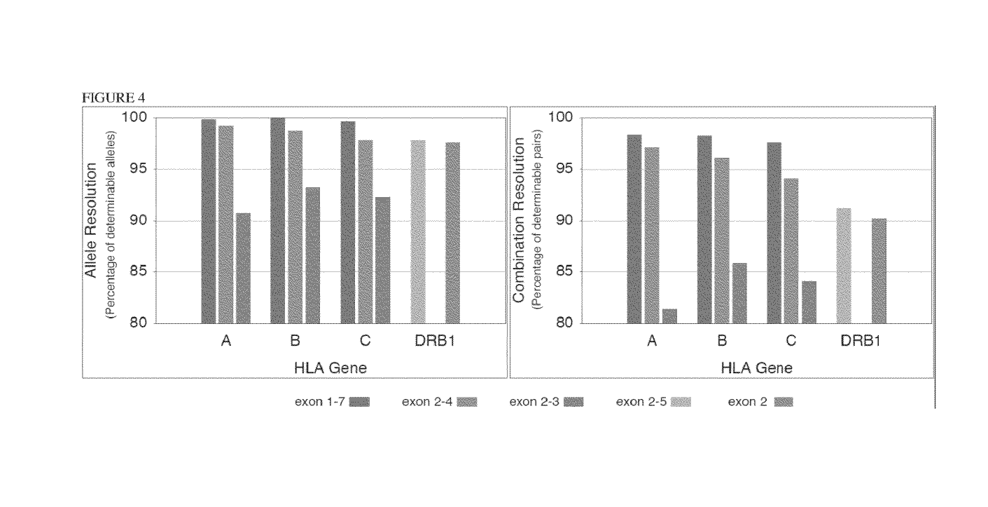

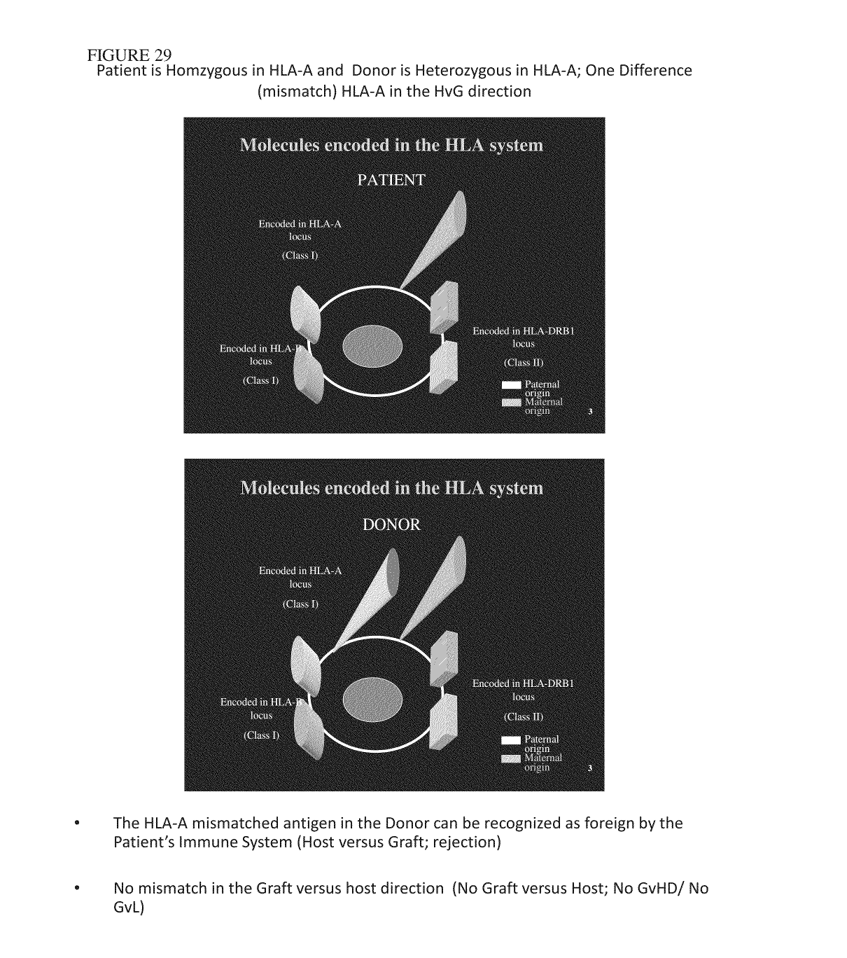

High-throughput HLA genotyping methodologies using massively parallel sequencing strategies such as Roche/454 sequencing generally amplify separately a few polymorphic exons and sequence in a multiplexed manner. In contrast, the present methods amplify a large genomic region of each gene including introns and the most polymorphic exons in a single PCR reaction and sequenced with a large excess of independent paired-end reads. There are two major ambiguities which arise from conventional SBT methods for HLA genotyping: incomplete-sequencing ambiguities that are commonly seen in typing protocols where alleles vary outside the targeted regions, and combination ambiguities that are frequently encountered where different allele combinations yield the same sequence pattern. In one non-limiting example, our lab used population frequency data and we do not resolve some genotype ambiguities with ratios greater than 1000:1 (FIG. 29). As more exons of a gene were sequenced, our method (FIG. 4), which sequenced exons 1 to 7 for HLA class I genes and exons 2 to 5 for HLA-DRB1, substantially enhanced the allele resolution and dramatically improved the combination resolution in comparison to the conventional SBT method, which sequences exons 2 and 3 for HLA class I genes and exon 2 alone for HLA-DRB1. In addition, the extensive sequence coverage allowed us to largely overcome genotype calling artifacts. The paired end sequencing strategy extends the read length effectively to 400-500 bases, which matches that of the Roche/454 platform, while allowing much higher throughput.

The linkage across 400 bases from paired-end reads, together with polymorphic sites in intron regions provided us with important phasing information and was useful to resolve combination ambiguities. We validated this long range PCR amplification and next-generation sequencing approach by re-typing the 40 different IHWG reference cell-lines. The accuracy of this approach was demonstrated with a high-degree (overall 99%) of concordance between our results and those reported in the reference databases. The Sanger sequencing data confirmed our genotype-calling results in the discordant alleles in all cell lines.

The methods disclosed herein can offer robust amplification, balance products and alleles, fully cover genomic regions, and accurately call genotypes. The data analysis can use solid and simple logic with minimal error; is accurate with a user-friendly interface for reviewing results; is fast and requires less than two hours for a Miseq run (12-24 samples); is able to pick up new alleles; can be used with a standalone desktop solution; and has the ability to generate assembly sequences. The data analysis logics include de-mutliplexing for identical barcodes at both ends of pair-end reads that lowers the chance of cross-contamination. The data analysis can use competitive mapping of all available reference sequences, including those form pseudo-genes are mapped and best alignments are passed. Data analysis can use filtering for best, identical (for cDNA only), and pair-end alignments. Data analysis uses genotype calling with a limited number of candidates (top 10 of each category: number of reads mapped, minimal coverage, minimal central coverage), enumerates the possible combination of homozygous and heterozygous sets, and ranks those combinations on aggregate number of reads mapped, minimal coverage, and minimal central coverage. The local de novo assembly can be performed to capture SNPs for novel alleles.

The approach can use the Illumina NGS platform and offers consistent performance with negligible errors. It has adaptability to both high and low throughput: low throughput with 16 to 24 samples for all loci in 5 days from sample to results; high throughput with 192 to 768 samples for all loci in 1 week from sample to results; and super-high throughput with 3072 samples for all loci in 2 weeks from sample to results. SGTC HLA typing offers full-automation for high throughput and semi-automation capability for low throughput. The highly-multiplexed NGS offers low cost not previously possible with Sanger-based SBT methods. SCTC HLA typing offers unique primer and PCR mix formulation with robust amplification of long range PCR, preservation of allele balance, and prevention of allele dropout. The unique library preparation uses fragmentase as opposed to Coveris shearing methodology to reduce cross contamination and blue pippen is used for size fractionation as opposed to beads based size fractionation to increase quality of final products for sequencing. SCTG HLA typing also interfaces with LIMS for sample tracking and effective lab workflow. Further refinements in progress include a filling reference database, sequence assembly after genotype assignment, and statistics of all reads utilized to make assignment.

The time to complete data analysis can be variable. Variation in time of analysis can depend on several factors. The data analysis pipeline, can take about 2 to about 3 hours for analysis against a cDNA reference sequences for one Miseq run (about 10 million reads). It can take about another hour to finish analysis against genomic reference data for one Miseq run. For Hiseq data, the yield of each lane is about 200 million reads. It can take about 2 days to complete the analysis. If 10 similar servers are available, this time can decreases to about 4 hours to complete the analysis.

The methods described herein allow for discovery of yet unidentified HLA alleles. Some non-limiting examples of alleles that this approach can identify can include: insertions, deletions, and substitutions. In some embodiments, the method of using PCR primers designed to hybridize to regions outside of polymorphic regions can increase the chance of capturing new alleles.

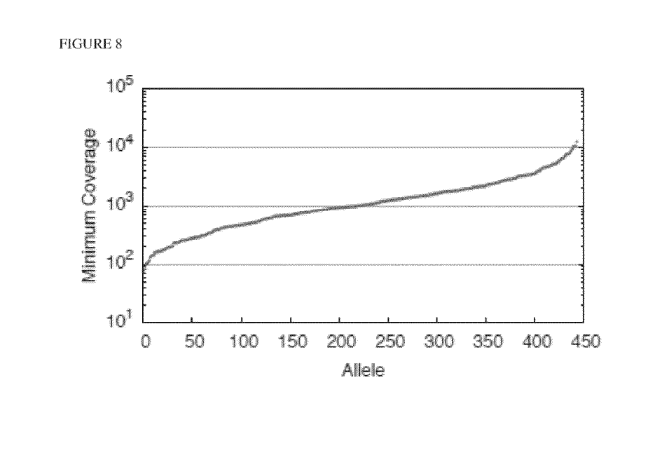

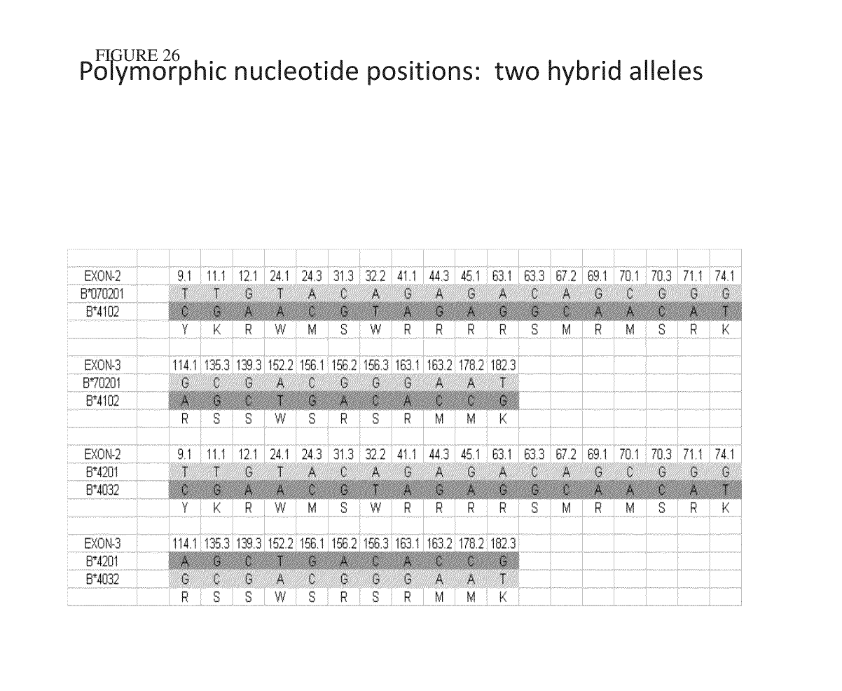

The methods described here can be further optimized to increase the number of samples on a single instrument run. In one non-limiting examples, HLA alleles from 59 clinical samples were typed in a single HiSeq2000 lane. 99.3% of alleles meet a coverage threshold of 100, and the majority of them were beyond a coverage threshold of 900 (FIG. 8). The ratios of minimum coverage of heterozygous alleles of a gene in the same sample were under four in all but two samples, indicating that heterozygous alleles of the same gene were amplified with similar efficiencies and coverage variation are largely due to pooling unevenness. One non-limiting simulation experiment showed that a minimum coverage of 20 could provide reliable information for genotype calling. For each sample, with 8 genes per sample, an average gene size of 5000, 2 diploids, 200 to achieve minimum coverage, 3 barcode variance, and 4 allege variance amounts to 192 million base pairs (FIG. 26). The HiSeq2000 produces about 200 million reads or 40,000 million by per lane and our experience suggest that 80% of reads are able to be mapped (FIG. 26). With an optimized protocol to improve the pooling evenness, we project that for HLA typing 4 genes, we can pool about 192 samples in one lane of Illumina HiSeq2000, or 2700 samples in one HiSeq2000 instrument run (15 lanes), respectively.

In some embodiments, we have demonstrated a successful approach for determining accurate HLA genotypes in a high-throughput manner for large numbers of clinical samples simultaneously. Having such a high throughput can effectively lower the cost per sample. Indeed, in the setting of testing many subjects simultaneously, the cost for high resolution typing by the novel methodology is significantly lower than classical Sanger sequencing and it in the same range or lower than the cost of probe-based assays, which have a much lower typing resolution. Therefore, the combination of high-resolution, high-throughput, and low cost will enable comprehensive disease-association studies with large cohorts. Broad coverage and deep sequencing offer great robustness of this method against PCR errors, PCR bias, sequencing errors, and sequencing bias et al. Paired-end sequencing amplify the difference of an authentic candidate with their many similar siblings. Complement logic (cDNA vs. genomic) and central read logic help to resolve difficult cases easily.

In some embodiments, HLA typing approaches described here can be useful in obtaining high-resolution HLA results of donors and cord blood units recruited or collected by registries of potential volunteer donors for bone marrow transplantation and cord blood banks. Successful outcomes of allogeneic hematopoietic stem cell transplantation can correlate well with close HLA matching between the patient and the selected donor unit. Also, in many diseases early treatment including hematopoietic stem cell transplantation soon after diagnosis, correlates with superior outcomes. Listing donors and units with the corresponding high resolution HLA type can dramatically accelerate the identification of optimally compatible donors.

In some embodiments, the methods of the invention can be adapted to accommodate the need for quick turnaround for urgent samples. With the Illumina Miseq, samples can be typed within about 1, 2, 3, 4, 5, 6, 7, 8, 9, or 10 days. In some embodiments, samples can be typed in less than five days. The typing method can be adapted to suit different sequencing platforms. For example, the alignment algorithms and HLA genotype calling can be independent of the sequencing method(s). The present study shows that the current knowledge of sequence variation in the HLA system can rapidly be expanded by the application of novel nucleotide sequencing technologies.

These data show an ability to analyze, comprehensively, segments of the HLA genes that have not been tested routinely. The testing of these areas gain insight into the fine details of the possible evolutionary pathways of the HLA variation. Furthermore, these methodologies allow refinement of the mapping of susceptibility factors, and of immunity-enabling features. In this regard, the approach can be extended to all HLA genes to discern patient-specific factors that may influence future vaccination strategies. Similarly, we may be able to obtain more precise evaluation of the HLA match grade between patients and unrelated donors in solid organ and hematopoietic stem cell transplantation.

### Materials and Methods

HLA typing reference cell-lines were obtained from the International Histocompatibility Working Group (IHWG) at the Fred Hutchinson Cancer Research Center. The SP reference panel was used for validating the Illumina HLA typing technology. The 47 clinical samples were drawn from the Molecular Genetics of Schizophrenia I linkage sample, which is part of the National Institute of Mental Health Center for Genetic Studies repository program. The other 12 clinical samples were from specimens of HSCT patients or donors that presented less common or novel allele types. Each clinical specimen was collected after subjects signed a written informed consent.

PCR primer design is as follows. To design gene-specific primers, we have analyzed all available sequences and chosen primers that would ensure the amplification of all known alleles for each gene. We have avoided regions of high variability, and where necessary, have designed multiple primers to ensure amplification of all alleles. For the class I HLA gene (HLA-A, -B, and -C), the forward primer was located in exon 1 near the first codon, and the reverse primer was located in exon 7. Only a limited number of genomic sequences were available for HLADRB1 genes. Therefore, the PCR primer for HLA-DRB1 genes were placed in less divergent exons. Taking into consideration the size of the PCR amplicons and completeness of genes, the forward primer for HLA-DRB1 was placed at the boundary between intron 1 and exon 2, and the reverse primer within exon 5. To ensure the robustness of the PCR reaction, the first exon of DRB1 was not included in order to avoid amplifying intron 1, which is about 8 kb in length.

Sample preparation is as follows. To amplify the selected HLA genes, individual long-range PCR reactions were performed using 5 pmol phosphorylated primers, 100 uM dNTPs, and 2.5 units Crimson LongAmp® Taq DNA Polymerase (New England Biolabs (NEB)) in a 25 μl reaction volume. The reaction included an initial denaturation at 94° C. for 2 min, followed by 40 cycles of 94° C. for 20 sec, 63° C. for 45 sec, and 68° C. for 5 min (for HLA-A, -B, -C) or 7 min for HLA-DRB1. The quality and the molecular weight of each PCR was estimated (assessed) in a 0.8% agarose gel and the approximate amount of each product was estimated by the pixel intensity of the bands. From the amplicon of each gene, approximately 300 ng were pooled and purified using Agencourt AMPure XP beads (Beckman Coulter Genomics) following the manufacturer's instructions, and subsequently ligated to form concatemers.

For the ligation reaction, overhangs generated by Crimson Taq Polymerase were removed by incubating the reaction with 3 units T4 polymerase (NEB), 2000 units T4 DNA Ligase (NEB) and 1 mM dNTP's in 10×T4 DNA ligase buffer for 10 minutes at room temperature. This was followed by the addition of 1 μl 50% PEG and incubated at room temperature for 30 minutes. Then another 2000 units of T4 DNA Ligase (NEB) was added followed by an overnight incubation at 4° C. After completion of the reaction, 1 μg of ligation product was randomly fragmented in a Covaris E210R (Covaris Inc) DNA shearing instrument to generate 300-350 bp fragments. 225 ng of fragmented DNA was end-repaired using the Quick blunting kit (NEB) followed by addition of deoxyadenosines, using Klenow polymerase, to facilitate addition of barcoded adaptors using 5000 units of Quick Ligase (NEB).

For multiplex processing, multiple samples were pooled together and purified using AMPure XP beads (Beckman Coulter). The samples were run on a Pippin Prep DNA size selection system (Sage Biosciences) to select 350-450 base pair fragments. After elution of the sample, one-half of the eluate was enriched by 13 cycles of PCR using Phusion Hot Start High Fidelity Polymerase (NEB). The enriched libraries were quantified, and the quality checked by an Agilent 2100 Bioanalyzer (Agilent Technologies, Santa Clara, Calif.). The libraries were diluted to a 10 nM concentration using elution buffer, EB (Qiagen). Following denaturation with sodium hydroxide, the amplified libraries were sequenced at a final concentration of 3.5 pM on the Illumina GAIIx instrument (Illumina Inc.) using 8 Illumina 36 cycle SBS sequencing kits (v5) to perform a paired-end, 2×150 bp, run. After sequencing, the resulting images were analyzed with the proprietary Illumina pipeline v1.3 software. Sequencing was done according to the manual from Illumina. To verify discordant calls or potential novel alleles, products from an independent PCR amplification were used to confirm the results by Sanger sequencing using the Big Dye Terminator Kit v3.1 (Life Technologies, Carlsbad, Calif.) and internal sequencing primers. 10 μl of PCR products were digested with 1 unit Shrimp Alkaline Phosphatase and 1.0 unit of Exonuclease I (Affymetrix Inc.) at 37° C. for 15 min followed by a 20 min heat inactivation at 80° C. The products were directly used in the sequencing reaction or cloned with a TOPO® XL PCR Cloning Kit with One Shot® TOP10 Electrocomp™ E. coli (Invitrogen) prior to sequencing on the 3730 instrument (Life Technologies).

Comparison of allele resolution and combination resolution when different regions were analyzed sequence-based typing (SBT) is considered the most comprehensive method for HLA typing. Due to technique difficulty and cost consideration, only the most polymorphic sites of HLA genes were analyzed by this method, which commonly uses the exon 2 and exon 3 sequences for HLA class I analysis and exon 2 alone for HLA class II analysis. With more and more new alleles discovered in the past several years, the accumulated data shown that besides those well-analyzed regions, other regions of HLA genes are polymorphic too. Because of this, IMGT/HLA data has designated new names for each group of HLA alleles that have identical nucleotide sequences across exons encoding the peptide binding domains (exon 2 and 3 for HLA class I and exon 2 for HLA class II) with an upper case ‘G’ which follows the three-field allele designation of the lowest numbered allele in the group.

To compare the allele resolution, which is defined as the percentage of alleles that can be resolved definitively when particular regions of a gene are analyzed, we counted the number of alleles which do not share the same sequence of the analyzed regions and calculated the percentage of those alleles overall all alleles listed in the IMGT/HLA database, which was released on Oct. 10, 2011. We applied the procedure if exons 1 to 7 (our method), or exons 2, 3, and 4, or exons 2 and 3 (conventional SBT methods) are determined for HLA class I genes, or exons 2 to 5 (our method) or exon 2 (conventional SBT methods) for HLA-DRB1. To compare the combination resolution, which is defined as the percentage of combinations of two heterozygous alleles that can be resolved definitively when particular regions of a gene are analyzed, we first enumerated the combined sequence pattern of the analyzed regions as if two heterozygous alleles were co-amplified and determined by Sanger sequencing method, and counted the number of combinations, each of which has a unique sequence pattern. We then calculated the percentage of those combinations of unique sequence pattern overall all enumerated combinations. We applied the procedure if exons 1 to 7 (our method), or exons 2, 3, and 4, or exons 2 and 3 (conventional SBT methods) are determined for HLA class I genes, or exons 2 to 5 (our method) or exon 2 (conventional SBT methods) for HLA-DRB1. For HLA-DRB1 genes, only 15% and 7% reference sequences cover exon 3 and 4 regions in the IMGT/HLA database released on Oct. 10, 2011. The procedure we employed did not count difference in exon 3 and 4 if there is no sequence information. Therefore, the difference between different methods over HLA-DRB1 cannot be clearly illustrated.

TABLE 1
Primers
direction of the primers 5 prime to 3 prime
HLA-A

| | |
|---|---|
| | Forward primers |
| (SEQ ID NO: 1) | TCCCCAGACGCCGAGGATGGCC |
| (SEQ ID NO: 2) | TCCCCAGACCCCGAGGATGGCC |
| (SEQ ID NO: 3) | CCTTGGGGATTCCCCAACTCCGCAG |
| | Reverse primers |
| (SEQ ID NO: 4) | CACATCAGAGCCCTGGGCACTGTC |
| (SEQ ID NO: 5) | TTATGCCTACACGAACACAGACACATG |

HLA-B

| | |
|---|---|
| | Forward primers |
| (SEQ ID NO: 6) | CTCCTCAGACGCCGAGATGCTG |
| (SEQ ID NO: 7) | CTCCTCAGACGCCAAGATGCTG |
| (SEQ ID NO: 8) | CTCCTCAGACACCGAGATGCTG |
| (SEQ ID NO: 9) | CTCCTCAGACGCCGAGATGCGG |
| (SEQ ID NO: 10) | CTCCTCAGACGCCAAGATGCGG |
| (SEQ ID NO: 11) | CTCCTCAGACACCGAGATGCGG |
| (SEQ ID NO: 12) | CCAACTTGTGTCGGGTCCTTCTTCCAGG |
| (SEQ ID NO: 13) | CCAACCTATGTCGGGTCCTTCTTCCAGG |
| | Reverse primers |
| (SEQ ID NO: 14) | CACATCAGAGCCCTGGGCACTGTC |
| (SEQ ID NO: 15) | CAT CCC TCT TTC TAO AGC AAC CCC CT |
| (SEQ ID NO: 16) | CAT CCC TCT TTC GAC AGC AAC CCC CT |

HLA-C

| | |
|---|---|
| | Forward primers |
| (SEQ ID NO: 17) | CTCCCCAGACGCCGAGATGCGG |
| (SEQ ID NO: 18) | CTCCCCAGAGGCCGAGATGCGG |
| (SEQ ID NO: 19) | GAGTCCAAGGGGAGAGGTAAGTTTCCT |
| (SEQ ID NO: 20) | GAGTCCAAGGGGAGAGGTAAGTGTCCT |
| | Reverse primers |
| (SEQ ID NO: 21) | CTCATCAGAGCCCTGGGCACTGTT |
| (SEQ ID NO: 22) | CTATCCCTCCTCCCACACCAACCG |

HLA-DQA

| | |
|---|---|
| | Forward primers |
| (SEQ ID NO: 23) | GCTCTTAATACAAACTCTTCAGCTAGTAACT |
| (SEQ ID NO: 24) | GCTCTTAATACAAACTCTTCAGCTAGTAACT |
| (SEQ ID NO: 25) | GCTCTTAATAGAAACTCTTCAACTAGTAACT |
| | Reverse primers |
| (SEQ ID NO: 26) | TCACAATGGCCCTTGGTGTCT |
| (SEQ ID NO: 27) | TCACAATGGCCCCTGGTGTCT |
| (SEQ ID NO: 28) | TCACAAGGGCCCTTGGTGTCT |

HLA-DQB

| | |
|---|---|
| | Forward primers |
| (SEQ ID NO: 29) | CCATCAGGTCCGAGCTGTGTTGACTACCACTT |
| (SEQ ID NO: 30) | CCATCAGGTCCGAGCTGTGTTGACTACCACTA |
| (SEQ ID NO: 31) | CCATCAGGTCCAAGCTGTGTTGACTACCACTA |
| (SEQ ID NO: 32) | CCATCAGGTCTGAGCTGTGTTGACTACCACTA |
| (SEQ ID NO: 33) | CCATCAGGTCCGAGCTGTGTTGACTACCACTG |
| | Reverse primers |
| (SEQ ID NO: 34) | CCTAGGGCAGAGCAGGGGGACAAGC |
| (SEQ ID NO: 35) | CCTAGGGCAGAGCAGGGAGACAAGC |
| (SEQ ID NO: 36) | CCTAGGGCAGAGCAGGGGGACAAGC |
| (SEQ ID NO: 37) | AGTCTTGATCCTCATAGCAGCAA |

HLA-DPA

| | |
|---|---|
| | Forward primers |
| (SEQ ID NO: 38) | ATGCAGCGGACCATGTGTCAACTTATGC |
| | Reverse primers |
| (SEQ ID NO: 39) | ACATTCCCACCTTTACAGTATTTCACAGG |

HLA-DPA

| | |
|---|---|
| | Forward primers |
| (SEQ ID NO: 40) | CGCCCCCTCCCCGCAGAGAATTA |
| | Reverse primers |
| (SEQ ID NO: 41) | ACCTTTCTTGCTCCTCCTGTGCATGAAG |
| HLA-DRB | Forward primers |
| (SEQ ID NO: 42) | TTCGTGTCCCCACAGCACGTTTC |
| (SEQ ID NO: 43) | TTCGTGTACCCGCAGCACGTTTC |
| (SEQ ID NO: 44) | TTCGTGTCCCCACAGCATGTTTC |
| (SEQ ID NO: 45) | TTCTTGTCCCCCCAGCACGTTTC |
| (SEQ ID NO: 46) | TTTGTGCCCCCACAGCACGTTTC |
| | Reverse primers |
| (SEQ ID NO: 47) | ACCTGTTGGCTGAAGTCCAGAGTGTC |
| (SEQ ID NO: 48) | ACCTCTTGGCTGAAGTCCAGAGTGTC |
| (SEQ ID NO: 49) | ACCTGTTGGCTGGAGTCCAGAGTGTC |
| (SEQ ID NO: 50) | ACCTGTTGGCGGAAGTCCAGAGTGTC |
| (SEQ ID NO: 51) | ACCTGTTGGGTGAAGTCCAGAGTGCC |

MIC-A

| | |
|---|---|
| | Forward primers |
| (SEQ ID NO: 52) | TGTGCGTTGGGGACAAGGCAATTCT |
| (SEQ ID NO: 53) | ACACATCGGAATCACCTAGGGAACT |
| (SEQ ID NO: 54) | GGGTAGAAGATGGTAGATGACAGCT |
| (SEQ ID NO: 55) | GTGGGGAAAGGACCCCGGTCCCTGC |
| | Reverse primers |
| (SEQ ID NO: 56) | ACCCTTACACTCTCTGCCATGACCA |
| (SEQ ID NO: 57) | AAACAGGGCCCAGCCAGGGTCCCTC |
| (SEQ ID NO: 58) | GTGCTGTGCAACAGATAATGACTGC |
| (SEQ ID NO: 59) | AGGAAGTGAAAAGTGGTCAAGCTGA |

MIC-B

| | |
|---|---|
| | Forward primers |
| (SEQ ID NO: 60) | TGCCACCGTCACCACTATCTACTTG |
| (SEQ ID NO: 61) | TACCATCAGGAAGGTTCAAACCATG |
| (SEQ ID NO: 62) | GGTAGAAGATGGTAGGTGATGGCTG |
| (SEQ ID NO: 63) | GAAATGGACACAGTTCCTGATCCTG |
| (SEQ ID NO: 64) | TCTCCCTGAAACCGCTTCTAAATGC |
| | Reverse primers |
| (SEQ ID NO: 65) | GTTGAGGGGAAGCCTTCTCTGTCAC |
| (SEQ ID NO: 66) | CTCCACACCCCTCTCCAGACACTGA |
| (SEQ ID NO: 67) | TTTATGTGGGGAAGGGAAGCCTTTA |
| (SEQ ID NO: 68) | AGTGAATGGGGAAGGAATGAGAGAC |

HLA A, B, C, DPA & DPB

| | Temp. | | Num. of |
|---|---|---|---|
| Number | (° c.) | Time (min) | Cycle |
| Denaturation | 94 | 3 min | 1 |
| Denaturation | 94 | 30 sec. | 37 |
| Extension | 68 | 6 min | |
| Extension | 68 | 10 min | 1 |
| | 4 | ∞ | 1 |

HLA QA2 & DRB

| | Temperature | | |
|---|---|---|---|
| | (° c.) | Time (min) | Cycle |
| Denaturation | 94 | 3 | 1 |
| Denaturation | 94 | 30 sec | 40 |
| Annealing | 63 | 1 | |
| Extension | 68 | 10 | |
| Final Extension | 68 | 10 | 1 |

HLA QA1 (Red Crimson)

| | Temperature | | |
|---|---|---|---|
| | (° c.) | Time (min) | Cycle |
| Denaturation | 94 | 3 | 1 |
| Denaturation | 94 | 30 sec | 40 |
| Annealing | 63 | 2 | |
| Extension | 68 | 8 | |
| Final | 68 | 10 | 1 |
| Extension | | | |

HLA DQB

| | Temperature | | |
|---|---|---|---|
| | (° c.) | Time (min) | Cycle |
| Denaturation | 94 | 3 | 1 |
| Denaturation | 94 | 30 sec | 40 |
| Annealing | 60 | 2 | |
| Extension | 68 | 8 | |
| Final | 68 | 10 | 1 |
| Extension | | | |

1. A method of high throughput genotyping comprising steps of:
(a) amplifying PCR product using a template nucleic acid and a mixture of regular dNTPs and a dNTP analog to generate an amplified PCR product;
(b) sequencing said amplified PCR product obtained in step (a), wherein deep sequencing data is generated by using independent paired-end reads; and
(c) using a first computing device to analyze said deep sequencing data.

2. The method of claim 1, wherein said template nucleic acid is an HLA gene.

3. The method of claim 1, wherein said template nucleic acid comprises one or more nucleic acids from the group consisting of: HLA-A, HLA-B, HLA-C, HLA-DQA and HLA-DQB.

4. The method of claim 1, wherein said template nucleic acid is a polymorphic genomic region.

5. The method of claim 4, wherein said amplified PCR product comprises said polymorphic genomic region.

6. The method of claim 1, wherein said method comprises long-range PCR of a polymorphic genomic region.

7. The method of claim 1, wherein said dNTP analog is selected from the group consisting of: 5-aminoallyl-2′-dCTP, (1-thio)-2′-dCTP, 5-methyl-2′-dCTP, 2-thio-2′-dCTP, 5-iodo-2′-dCTP, 2-amino-2′-dATP, 2-thio-TTP, 5-propynyl-2′-dCTP, N⁴-methyl-2′-dCTP, 7-deaza-2′-dATP, (1-thio)-2′dGTP, (1-thio)-2′-dATP, 5-bromo-2′-dCTP, and 7-deaza-dGTP.

8-10. (canceled)

11. The method of claim 1, wherein said step (a), prior to said amplifying, further comprises a step to hybridize a primer to a non-polymorphic region of said template nucleic acid.

12. The method of claim 11, wherein said primer comprises one or more dNTP analogs.

13. The method of claim 1, wherein said amplified long range PCR product is sequenced to a depth of at least 1000 reads per sequence.

14. The method of claim 1, wherein said step (b) further comprises, prior to said sequencing, a step to mix
(i) a first amplified long range PCR product obtained from a first template nucleic acid in step (a) with
(ii) a second amplified long range PCR product obtained from a second temple nucleic acid in step (a).

15. The method of claim 14, wherein a first mixing ratio between said first and second amplified long range PCR products is determined by a second computing device.

16. The method of claim 1, wherein a second mixing ratio between said dNTP analog and its corresponding regular dNTP is about 3:1.

17-27. (canceled)

28. A method for determining the genotype of an HLA gene in an individual, wherein said method comprises steps of:
(a) amplifying multiple exons and intervening introns of said HLA gene in a single long range PCR reaction, wherein said single long range PCR reaction uses a mixture of regular dNTPs and a dNTP analog to generate an amplified HLA gene;
(b) deep sequencing said amplified HLA gene obtained in step (a); and
(c) performing deconvolution analysis to determine a genotype of each allele of said HLA gene.

29. The method of claim 28, wherein said HLA gene is an HLA class I gene.

30. The method of claim 29, wherein said HLA class I gene comprises one or more genes selected from the group consisting of: HLA-A, HLA-B and HLA-C.

31. The method of claim 28, wherein said HLA gene is an HLA class II gene.

32. The method of claim 31, wherein said HLA class II gene comprises one or more genes selected from the group consisting of: HLA-DQA and HLA-DQB.

33. The method of claim 28, wherein a mixing ratio between said dNTP analog and its corresponding regular dNTP is about 3:1.

34. The method of claim 28, wherein said dNTP analog is selected from the group consisting of: 5-aminoallyl-2′-dCTP, (1-thio)-2′-dCTP, 5-methyl-2′-dCTP, 2-thio-2′-dCTP, 5-iodo-2′-dCTP, 2-amino-2′-dATP, 2-thio-TTP, 5-propynyl-2′-dCTP, N⁴-methyl-2′-dCTP, 7-deaza-2′-dATP, (1-thio)-2′dGTP, (1-thio)-2′-dATP, 5-bromo-2′-dCTP, and 7-deaza-dGTP.

35. (canceled)

36. The method of claim 28, wherein said step (a) further comprises a step of performing a nested PCR to amplify a genomic area covering at least 4 exons of said HLA gene, wherein said nested PCR amplifies all introns and said at least 4 exons in said long range PCR reaction with at least one set of target-specific primers that hybridize to sequences flanking said HLA gene.

37. The method of claim 36, wherein in said step (b), prior to said sequencing, said amplified HLA gene is fragmented and ligated to second primers to generate a fragmented and barcoded amplified HLA gene, said second primers comprising
(a) a target specific identifier for said individual;
(b) a target specific identifier for said HLA gene, and
(c) a sequencing adaptor.

38. (canceled)

39. The method of claim 37, wherein said fragmented and barcoded amplified HLA gene is sequenced to a depth of at least 1000 reads per sequence.

40. The method of claim 39, wherein said deconvolution analysis in step (c) is performed by mapping said sequence reads to a chromatid.

41. The method of claim 40, wherein said mapping comprises the steps of:
(a) aligning said sequence reads to a database of reference sequences;
(b) counting a number of central reads;
(c) computing a minimum coverage of overall reads;
(d) computing a minimum coverage of central reads for each of said reference sequences;
(e) enumerating all combinations of homozygous alleles or heterozygous alleles of said HLA gene and counting distinct reads that map to each said combination; and
(f) assigning a genotype to said combination with the maximum number of distinct reads; wherein steps (a)-(f) are embodied as a first program of instructions executable by a first computer and performed by means of first software components loaded into said first computer.

42. The method of claim 41, further comprising a step of performing de novo assembly of said sequence reads, wherein said step of performing de novo assembly of said sequence reads comprises the steps of:
(a) partitioning said sequence reads which include unmapped regions into short tiled fragments with a one base offset;
(b) building a directed weighted graph wherein each said short tiled fragment is represented as a node and two consecutive short tiled fragments of the same said sequence read are connected, and an edge between two said nodes is weighted with the frequency of said sequence reads from the said two connected nodes;
(c) constructing a contig using a path with the maximum sum of weights; and
(d) comparing said contig with its corresponding closest reference sequence;
wherein steps (a)-(d) are embodied as a second program of instructions executable by a second computer and performed by means of second software components loaded into said second computer.

43. The method of claim 42, further comprising a step of parsing alignments, wherein said step of parsing alignments comprises the steps of:
(a) filtering to keep a first alignment with best bit-scores;
(b) eliminating a second alignment containing either mismatches or gaps;
(c) deleting a third alignment shorter than 50 bases in length if first corresponding exons of said third alignment are longer than 50 bases, and removing a
(d) fourth alignment shorter than second corresponding exons of said fourth alignment if said second corresponding exons are less than 50 bases in length; and
(e) removing a fifth alignment in which said reference sequence for said fifth alignment is mapped to only one end of a paired-end read, wherein at least one of said references is mapped to both ends of said paired-end read.

44. The method of claim 28, wherein said individual is a human.

45. The method of claim 44, wherein said HLA gene comprises HLA-A, HLA-B and HLA-C, and wherein the genotypes of said HLA-A, said HLA-B and said HLA-C are determined.

46. The method of claim 45, wherein exons 1 to 7 are amplified.

47. The method of claim 28, wherein said HLA gene comprises HLA-DQA and HLA-DQB, and wherein the genotypes of said HLA-DQA and said HLA-DQB are determined.

48. The method of claim 47, wherein exons 1-4 are amplified.

49. The method of claim 28, wherein when a first amplified HLA gene is obtained from a first HLA gene in step (a) and a second amplified HLA gene is obtained from a second HLA gene in step (a), said step (b) further comprising the steps of:
(a) computing a mixing ratio between said first and second amplified HLA genes; and
(b) adding said first and second amplified HLA genes according to said mixing ratio in a single deep sequencing process.

50-70. (canceled)
